(19) United States
(12) Patent Application Publication
(10) Pub. No.: US 2009/0246134 A1
(43) Pub. Date: Oct. 1, 2009

(54) COMPOSITIONS AND METHODS FOR MODULATING GATED ION CHANNELS

(75) Inventors: Rahul Vohra, Ottawa (CA); Gilles Dube, Montreal (CA); Zhonghong Gan, Ottawa (CA)

Correspondence Address: LAHIVE & COCKFIELD, LLP;FLOOR 30, SUITE 3000, ONE POST OFFICE SQUARE, BOSTON, MA, 02109, US

(73) Assignee: PainCeptor Pharma Corporation, St. Laurent (CA)

(21) Appl. No.: 12/130,461

(22) Filed: May 30, 2008

Related U.S. Application Data
(60) Provisional application No. 60/932,490, filed on May 30, 2007.

(51) Int. Cl. A61K 49/00; C07D 471/04; C07D 498/04

(52) U.S. Cl. 424/9.1; 546/84; 544/66

(57) ABSTRACT

The present invention is directed toward radiolabelled ASIC imaging agents, as well as metabolites of ASIC antagonists. These compounds are useful for the diagnosis and treatment of diseases and disorders related to the activity of gated ion channels.

### RELATED APPLICATION

This application claims priority to U.S. Provisional Application No. 60/932,490, Attorney Docket No. PCI-058-1, filed May 30, 2007, titled “COMPOSITIONS AND METHODS FOR MODULATING GATED ION CHANNELS.” The contents of any patents, patent applications, and references cited throughout this specification are hereby incorporated by reference in their entireties.

### TECHNICAL FIELD

The present invention relates to compositions which modulate the activity of gated ion channels and methods and uses thereof.

### BACKGROUND

Mammalian cell membranes are important to the structural integrity and activity of many cells and tissues. Of particular interest is the study of trans-membrane gated ion channels which act to directly and indirectly control a variety of pharmacological, physiological, and cellular processes. Numerous gated ion channels have been identified and investigated to determine their roles in cell function.

Gated ion channels are involved in receiving, integrating, transducing, conducting, and transmitting signals in a cell, e.g., a neuronal or muscle cell. Gated ion channels can determine membrane excitability. Gated ion channels can also influence the resting potential of membranes, waveforms, and frequencies of action potentials, and thresholds of excitation. Gated ion channels are typically expressed in electrically excitable cells, e.g., neuronal cells, and are multimeric. Gated ion channels can also be found in nonexcitable cells (e.g., adipose cells or liver cells), where they can play a role in, for example, signal transduction.

Among the numerous gated ion channels identified to date are channels that are responsive to, for example, modulation of voltage, temperature, chemical environment, pH, ligand concentration and/or mechanical stimulation. Examples of specific modulators include: ATP, capsaicin, neurotransmitters (e.g., acetylcholine), ions, e.g., Na⁺, Ca⁺, K⁺, Cl⁻, H⁺, Zn⁺, Cd⁺, and/or peptides, e.g., FMRFamide. Examples of gated ion channels responsive to these stimuli are members of the DEG/ENaC, TRP and P2X gene superfamilies.

Members of the DEG/ENaC gene superfamily show a high degree of functional heterogeneity with a wide tissue distribution that includes transporting epithelia as well as neuronal excitable tissues. DEG/ENaC proteins are membrane proteins which are characterized by two transmembrane spanning domains, intracellular N- and C-termini and a cysteine-rich extracellular loop. Depending on their function in the cell, DEG/ENaC channels are either constitutively active like epithelial sodium channels (ENaC) which are involved in sodium homeostasis, or activated by mechanical stimuli as postulated for C. elegans degenerins, or by ligands such as peptides, as is the case for FaNaC from Helix aspersa which is a FMRFamide peptide-activated channel and is involved in neurotransmission, or by protons as in the case for the acid sensing ion channels (ASICs). For a recent review on this gene superfamily see Kellenberger, S. and Schild, L. (2002) Physiol. Rev. 82:735, incorporated herein by reference.

There are seven presently known members of the P2X gene superfamily; P2X₁ (also known as P2RX1), P2X₂ (also known as P2RX2), P2X₃ (also known as P2RX3), P2X₄ (also known as P2RX4), P2X₅ (also known as P2RX5), P2X₆ (also known as P2RX6), and P2X₇ (also known as P2RX7). P2X protein structure is similar to ASIC protein structure in that they contain two transmembrane spanning domains, intracellular N- and C-termini and a cysteine-rich extracellular loop. All P2X receptors open in response to the release of extracellular ATP and are permeable to small ions and some have significant calcium permeability. P2X receptors are abundantly distributed on neurons, glia, epithelial, endothelia, bone, muscle and hematopoietic tissues. For a recent review on this gene superfamily, see North, R. A. (2002) Physiol. Rev. 82:1013, incorporated herein by reference.

To date, the transient receptor potential (TRP) superfamily consists of 28 non-selective cation channels subdivided into six main subfamilies: the TRPC (canonical), TRPV (vanilloid), TRPM (melastatin), TRPP (polycystin), TRPML (mucolipin), and the TRPA (ankyrin) groups. The great majority of functionally characterized TRP channels are permeable to Ca²⁺. The TRP channels are widely distributed and participate in various cellular functions. Although our understanding of the physiological and pathophysiological involvement of many of these channels is limited, evidence exists (e.g., changes in expression levels) that link some of these channels to several diseases. TRP channels play also a role in some systemic reactions and diseases provoked by specific irritants, inflammation mediators, and foreign toxins.

The receptor expressed in sensory neurons that reacts to the pungent ingredient in chili peppers to produce a burning pain is the capsaicin (TRPV or vanilloid) receptor, denoted TRPV1 (also known as VR1, TRPV1alpha, TRPV1beta). The TRPV1 receptor forms a nonselective cation channel that is activated by capsaicin and resiniferatoxin (RTX) as well as noxious heat (>43° C.), with the evoked responses potentiated by protons, e.g., H⁺ ions. Acid pH is also capable of inducing a slowly inactivating current that resembles the native proton-sensitive current in dorsal root ganglia. Expression of TRPV1, although predominantly in primary sensory neurons, is also found in various brain nuclei and the spinal cord (Physiol. Genomics 4:165-174, 2001).

Two structurally related receptors, TRPV2 (also known as VRL1 and VRL) and TRPV4 (also known as VRL-2, Trp12, VROAC, OTRPC4), do not respond to capsaicin, acid or moderate heat but rather are activated by high temperatures (Caterina, M. J., et al. (1999) Nature. 398(6726):436-41). In addition, this family of receptors, e.g., the TRPV or vanilloid family, contains the ECAC-1 (also known as TRPV5 and CAT2, CaT2) and ECAC-2 (also known as TRPV6, CaT, ECaC, CAT1, CATL, and OTRPC3) receptors which are calcium selective channels (Peng, J. B., et al. (2001) Genomics 76 (1-3):99-109). For a recent review of TRPV (vanilloid) receptors, see Nilius, B. et al. (2007), Physiol. Rev. 87: 165-217, incorporated herein by reference.

Other pungent substances have also been reported to evoke noxious sensations different to that of capsaicin. This allows for the identification of other mammalian members of the TRP surperfamily. TRPA1 (ANKTM1) is the only member of the TRPA subfamily is expressed by a subset of TRPV1-positive Aδ and C primary sensory fibers (those sensory fibers are involved for thermo-, mechano-, and chemo-sensory transduction). TRPA1 is activated by pungent substances such as mustard oil, allicin (pungent ingredient in garlic), or cinnamaldehyde, which leads to a burning pain sensation similar to the effect of capsaicin on TRPV1 (Bandell et al. (2004) Neuron 41:849-857). However, unlike TPRV1, which is activated by heat, TRPA1 is activated by noxious cold (<17° C.) (Wang and Woolf (2005) Neuron 46:9-12). Several recent reports indicate that TRPA1 is involved in the development of chronic cold hyperalgesia associated with inflammation or nerve damage (e.g., Bandell et al. (2004) Neuron 41:849-857). NGF which is release during inflammation and nerve injury was found to up-regulate TRPA1 (Diogenes et al (2007) J. Dent. Res. 86:550-555). TRPA1 may also be the receptor responsible for the pain mediated by formalin (McNamara et al. (2007) PNAS 104: 13525-13530).

TRPM8 (or Cold-Menthol Receptor 1; CMR1) is expressed in a subset of small diameter DRG neurons that does not express TRPV1 (although some overlap has been shown). TPRM8 is the 8^{th} member of the TPRM family and like TRPA1 is activated by cold and menthol and icilin, two substances that produces cold sensation. However, unlike TRPA1, TRPM8 is activated by innocuous cold (<30° C.). Recent reports on TRPM8 knockout mice demonstrate that TRPM8 could plays a significant role in certain cold types of cold-induced pain in human (Colburn et al. (2007) Neuron 54:379-386; Dhaka et al. (2007) Neuron 54:371-378).

Research has shown that ASICs play a role in pain, neurological diseases and disorders, gastrointestinal diseases and disorders, genitourinary diseases and disorders, and inflammation. For example, it has been shown that ASICs play a role in pain sensation (Price, M. P. et al., Neuron. 2001; 32 (6): 1071-83; Chen, C. C. et al., Neurobiology 2002; 99 (13) 8992-8997), including visceral and somatic pain (Aziz, Q., Eur. J. Gastroenterol. Hepatol. 2001; 13 (8):891-6); chest pain that accompanies cardiac ischemia (Sutherland, S. P. et al. (2001) Proc Natl Acad Sci USA 98:711-716), and chronic hyperalgesia (Sluka, K. A. et al., Pain. 2003; 106 (3):229-39). ASICs in central neurons have been shown to possibly contribute to the neuronal cell death associated with brain ischemia and epilepsy (Chesler, M., Physiol. Rev. 2003; 83: 1183-1221; Lipton, P., Physiol. Rev. 1999; 79:1431-1568). ASICs have also been shown to contribute to the neural mechanisms of fear conditioning, synaptic plasticity, learning, and memory (Wemmie, J. et al., J. Neurosci. 2003; 23 (13):5496-5502; Wemmie, J. et al., Neuron. 2002; 34 (3):463-77). ASICs have been shown to be involved in inflammation-related persistent pain and inflamed intestine (Wu, L. J. et al., J. Biol. Chem. 2004; 279 (42):43716-24; Yiangou, Y., et al., Eur. J. Gastroenterol. Hepatol. 2001; 13 (8): 891-6), and gastrointestinal stasis (Holzer, Curr. Opin. Pharm. 2003; 3: 618-325). Recent studies done in humans indicate that ASICs are the primary sensors of acid-induced pain (Ugawa et al., J. Clin. Invest. 2002; 110: 1185-90; Jones et al., J. Neurosci. 2004; 24: 10974-9). Furthermore, ASICs are also thought to play a role in gametogenesis and early embryonic development in Drosophila (Darboux, I. et al., J. Biol. Chem. 1998; 273 (16):9424-9), underlie mechanosensory function in the gut (Page, A. J. et al. Gastroenterology. 2004; 127 (6):1739-47), and have been shown to be involved in endocrine glands (Grunder, S. et al., Neuroreport. 2000; 11 (8): 1607-11).

Therefore, compounds that modulate these gated ion channels would be useful in the treatment of diseases and disorders.

### SUMMARY OF THE INVENTION

The current invention is related to the gated ion channel-modulating agents disclosed in WO 2007/059,608, which are useful for the treatment of gated ion channel-diseases and disorders, such as pain. In particular, this invention is directed toward metabolites of those gated ion channel-modulating agents, as well as use of those gated ion channel-modulating agents as imaging agents.

This invention relates to gated ion channel-targeting imaging agents, including ASIC imaging agents of the Formula I, as well as compounds that are mammalian metabolites that have the structures of Formula III. In a particular embodiment, the invention is directed toward mammalian metabolites of the gated ion channel modulators 5-(5-fluoro-2-methoxyphenyl)-6,7,8,9-tetrahydro-3-(hydroxyimino)-8-(methyl)-1H-pyrrolo[3,2-h]isoquinoline-2(3H)-one (Compound A) or 5-(5-fluoro-2-methoxyphenyl)-6,7,8,9-tetrahydro-3-(hydroxyimino)-8-(ethyl)-1H-pyrrolo[3,2-h]isoquinoline-2(3H)-one (Compound B). In one embodiment, a compound of the invention is Compound L, which is a metabolite of the Compound A. These compounds are referred to herein as “the compounds of the invention.”

One aspect of the invention relates to pharmaceutical compositions comprising imaging agents of the Formula I, as well as metabolites of Compound A and Compound B, or optical or geometric isomers thereof, or a pharmaceutically acceptable salt, N-oxide, ester, quaternary ammonium salt thereof and a pharmaceutically acceptable carrier, vehicle or diluent thereof.

Another aspect of the invention relates to methods of treating diseases, e.g., pain, comprising administering an effective amount of imaging agents of the Formula I, or a compound of the Formula III (e.g., metabolites of Compound A and Compound B), or pharmaceutically acceptable salts, N-oxides, esters, or quaternary ammonium salts thereof.

In yet another aspect, the present invention provides for kits for use by a consumers to treat disease. The kit comprises a) an imaging agent of the Formula I, or a compound of Formula III (e.g., metabolites of Compound A and Compound B); and, optionally, b) instructions describing a method of using the imaging agent or metabolite to treat disease. The instructions may also indicate that the kit is for treatment of disease while substantially reducing the concomitant liability of adverse effects associated with the compound.

In another aspect, the invention provides an ASIC imaging agent of the Formula I, and pharmaceutically acceptable salts, enantiomers, stereoisomers, rotamers, tautomers, diastereomers, or racemates thereof, wherein at least one of the atoms of Formula I is an isotope.

In one embodiment, the ASIC imaging agent of the Formula I is represented by the Formula II, and pharmaceutically acceptable salts, enantiomers, stereoisomers, rotamers, tautomers, diastereomers, or racemates thereof, wherein at least one of the atoms of Formula II is an isotope.

In one embodiment, the isotope is selected from the group consisting of ²H, ³H, ¹³C, ¹⁴C, ¹⁵N, ¹⁸O, ¹⁷O ³¹P, ³²P, ³⁵S, ¹⁸F, ³⁶Cl, ¹²³I, ¹²⁵I, ¹²⁷I, ¹²⁹I, ¹³⁰I and ¹³¹I. In another embodiment, the isotope is selected from the group consisting of ³H and ¹⁴C.

In another embodiment, the ASIC imaging agent is an ion channel-targeting agent. In still another embodiment, the ion channel is comprised of at least one subunit selected from the group consisting of a member of the DEG/ENaC, P2X, and TRP gene superfamilies. In still another embodiment, the ion channel is comprised of at least one subunit selected from the group consisting of αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6, TRPA1 and TRPM8. In yet another embodiment, the ion channel is comprised of at least one subunit selected from the group consisting of ASIC1a and ASIC3.

In another aspect, the invention provides a method for diagnosing an ion channel-related condition in a patient, comprising administering an ion channel-targeting imaging agent to said patient and said ion channel-targeting imaging agent is imaged in said patient to determine the activity or amount of an ion channel in said patient. In one embodiment, the ion channel-targeting imaging agent is an imaging agent of the Formula I, wherein at least one of the atoms of Formula I is an isotope. In another embodiment, the ion channel-related condition is selected from the group consisting of pain, inflammation, cardiovascular disorders, respiratory conditions, genitourinary disorders, gastrointestinal disorders, cancers and neurological disorders.

In one embodiment, the pain is selected from the group consisting of inflammatory pain (e.g., osteoarthritis), neuropathic pain (e.g., PHN, diabetic neuropathies), visceral pain (e.g., pancreatitis), post surgical pain and bone cancer pain. In another embodiment, the cardiovascular disorder is selected from the group consisting of ischemic pain (e.g., intermittent claudication), cardiac ischemia and heart failure. In one embodiment, the respiratory condition is selected from the group consisting of asthma and COPD. In still another embodiment, the genitourinary disorder is selected from the group consisting of interstitial cystitis and overactive bladder. In another embodiment, the gastrointestinal disorder is selected from the group consisting of inflammatory bowel disease and neurological disorders (e.g., stroke and brain ischemia, peripheral neuropathies, and anxiety). In another embodiment, the cancer is bone cancer.

In another aspect, the invention provides a method for imaging ion channel-activity in a patient, comprising administering an ion channel-targeting imaging agent to said patient and imaging said ion channel-targeting imaging agent in said patient to determine the activity or amount of an ion channel in said patient. In one embodiment, the ion channel-targeting imaging agent is an imaging agent of the Formula I, wherein at least one of the atoms of Formula I is an isotope.

In another aspect, the invention provides a method for diagnosing pain, inflammation, cardiovascular disorders, respiratory conditions, genitourinary disorders, gastrointestinal disorders, or neurological disorders in a patient, comprising administering an imaging agent of Formula I to said patient and said imaging agent is imaged in said patient to determine the presence of one or more of these conditions.

In another aspect, the invention provides a method for imaging a tumor on or in a mammalian tissue inflicted with a tumor comprising contacting the mammalian tissue with an effective amount of an imaging agent of Formula I, and detecting the presence of the imaging agent.

In another aspect, the invention provides a method for imaging a tumor in a subject inflicted with a tumor comprising administering to the mammal an effective amount of an agent of Formula I, and detecting the presence of the imaging agent. In one embodiment, the tumor is located in the bone of a subject.

In another aspect, the invention provides a method for stratifying disease severity or prognosis in a patient, comprising administering an imaging agent of Formula I to said patient and said imaging agent is imaged in said patient.

In another aspect, the invention provides a kit for preparing a radiopharmaceutical preparation, said kit comprising an imaging agent of Formula I and instructions for the preparation and use of the imaging agent in the imaging of ion channel activity or an ion channel-related condition.

In another aspect, the invention provides a compound of Formula III, which is a metabolite of ASIC antagonists, e.g., metabolites of 5-(5-fluoro-2-methoxyphenyl)-6,7,8,9-tetrahydro-3-(hydroxyimino)-8-(methyl)-1H-pyrrolo[3,2-h]isoquinoline-2(3H)-one (Compound A) or 5-(5-fluoro-2-methoxyphenyl)-6,7,8,9-tetrahydro-3-(hydroxyimino)-8-(ethyl)-1H-pyrrolo[3,2-h]isoquinoline-2(3H)-one (Compound B), and pharmaceutically acceptable salts, enantiomers, stereoisomers, rotamers, tautomers, diastereomers, or racemates thereof.

In one embodiment, the compound of Formula III is represented by Compound L, which is a metabolite of Compound A.

In another aspect, the invention provides a method of treating pain in a subject in need thereof, comprising administering to the subject an effective amount of a compound of Formulae I, II, III or IV. In one embodiment, the pain is selected from the group consisting of cutaneous pain, somatic pain, visceral pain, neuropathic pain, acute pain and chronic pain.

In another aspect, the invention provides a method of treating an inflammatory disorder in a subject in need thereof, comprising administering to the subject an effective amount of a compound of Formulae I, II, III or IV. In one embodiment, the inflammatory disorder is inflammatory disorder of the musculoskeletal and connective tissue system, the respiratory system, the circulatory system, the genitourinary system, the gastrointestinal system or the nervous system.

In still another aspect, the invention provides a method of treating a neurological disorder in a subject in need thereof, comprising administering an effective amount of a compound of Formulae I, II, III or IV. In one embodiment, the neurological disorder is selected from the group consisting of schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, shingles, Huntington's chorea, Parkinson disease, anxiety disorders, panic disorders, phobias, anxiety hyteria, generalized anxiety disorder, and neurosis.

In another aspect, the invention provides a method of treating a disease or disorder associated with the genitourinary and/or gastrointestinal systems of a subject in need thereof, comprising administering to the subject an effective amount of a compound of Formulae I, II, III or IV. In one embodiment, the disease or disorder of the gastrointestinal system is selected from the group consisting of gastritis, duodenitis, irritable bowel syndrome, colitis, Crohn's disease, ulcers and diverticulitis. In another embodiment, the disease or disorder of the genitourinary system is selected from the group consisting of cystitis, urinary tract infections, glomerulonephritis, polycystic kidney disease, kidney stones and cancers of the genitourinary system.

### BRIEF DESCRIPTION OF THE DRAWINGS

FIG. 1 is a product ion spectrum of Compound A.

FIG. 2 is a product ion spectrum of Compound B.

FIG. 3 is a representative HPLC chromatogram of the Z-isomer of Compound A.

### DETAILED DESCRIPTION OF THE INVENTION

The present invention is based, at least in part, on the identification of compounds useful in modulation of the activity of gated ion channels. Gated ion channels are involved in receiving, conducting, and transmitting signals in a cell (e.g., an electrically excitable cell, for example, a neuronal or muscle cell). Gated ion channels can determine membrane excitability (the ability of, for example, a cell to respond to a stimulus and to convert it into a sensory impulse). Gated ion channels can also influence the resting potential of membranes, waveforms and frequencies of action potentials, and thresholds of excitation. Gated ion channels are typically expressed in electrically excitable cells, e.g., neuronal cells, and are multimeric; they can form homomultimeric (e.g., composed of one type of subunit), or heteromultimeric structures (e.g., composed of more than one type of subunit). Gated ion channels can also be found in nonexcitable cells (e.g., adipose cells or liver cells), where they can play a role in, for example, signal transduction.

Gated ion channels that are the focus of this invention are generally homomeric or heteromeric complexes composed of subunits, comprising at least one subunit belonging to the DEG/ENaC, TRP and/or P2X gene superfamilies. Non-limiting examples of the DEG/ENaC receptor gene superfamily include epithelial Na⁺ channels, e.g., αENaC, βENaC, γENaC, and/or δENaC, and the acid sensing ion channels (ASICs), e.g., ASIC1, ASIC1a, ASIC1b, ASIC2, ASIC2a, ASIC2b, ASIC3, ASIC4, BLINaC and/or hINaC. Non-limiting examples of the P2X receptor gene superfamily include P2X₁, P2X₂, P2X₃, P2X₄, P2X₅, P2X₆, and P2X₇. Non-limiting examples of the TRP receptor gene superfamily include TRPV1 (also referred to as VR1), TRPV2 (also referred to as VRL-1), TRPV3 (also referred to as VRL-3), TRPV4 (also referred to as VRL-2), TRPV5 (also referred to as ECAC-1), TRPV6 (also referred to as ECAC-2), TRPA1, and/or TRPM8.

Non limiting examples of heteromultimeric gated ion channels include αENaC, βENaC and γENaC; αENaC, βENaC and δENaC; ASIC1a and ASIC2a; ASIC1a and ASIC2b; ASIC1a and ASIC3; ASIC1b and ASIC3; ASIC2a and ASIC2b; ASIC2a and ASIC3; ASIC2b and ASIC3; ASIC1a, ASIC2a and ASIC3; ASIC3 and P2X, e.g. P2X₁, P2X₂, P2X₃, P2X₄, P2X₅, P2X₆ and P2X₇, preferably ASIC3 and P2X₂; ASIC3 and P2X₃; and ASIC3, P2X₂ and P2X₃ ASIC4 and at least one of ASIC1a, ASIC1b, ASIC2a, ASIC2b, and ASIC3; BLINaC (or hINaC) and at least one of ASIC1a, ASIC1b, ASIC2a, ASIC2b, ASIC3, and ASIC4; δENaC and ASIC, e.g. ASIC1a, ASIC1b, ASIC2a, ASIC2b, ASIC3 and ASIC4; P2X₁ and P2X₂, P2X₁ and P2X₅, P2X₂ and P2X₃, P2X₂ and P2X₆, P2X₄ and P2X₆, TRPV1 and TRPV2, TRPV5 and TRPV6, TRPV1 and TRPV4.

The physiological and pathophysiological function of ASICs or their subunit composition in different cells and tissues are poorly understood, however, their location (mostly neuronal) and gating properties make them an attractive candidate to serve as an acid sensor for pH nociception to convey pain sensation during conditions such as inflammation, ischemia, hematomas infection and other condition known to produce tissue acidosis.

Knowledge of the tissue, cellular, and subcellular distributions of these channels can provide important clues as to their function. In the peripheral nervous system (PNS) under physiological conditions, ASICs subunits are found in primary sensory neurons that innervate the skin (Price M P, et al. (2000) Nature 407:1007-1011; Price M P, et al. (2001) Neuron 32:1071-1083), heart (Benson C J, et al. (1999) Circ Res 84:921-928), gastrointestinal tracks (Page A J, et al. (2005b) Gut 54:1408-1415; Page A J, et al. (2004) Gastroenterology 127:1739-1747; Page A J, et al. (2005a) Gut 54:1408-1415), and muscles (Molliver D C, et al. (2005) Mol Pain 1:35). ASICs subunits are also found in the eye (Ettaiche M, et al. J Neurosci 24:1005-1012), ear (Hildebrand M S, et al. (2004) Hear Res 190:149-160), tongue (Ugawa S (2003) Anat Sci Int 78:205-210; Ugawa S, et al. J Neurosci 23:3616-3622), lungs (Gu Q et al. (2006) Am J Physiol Lung Cell Mol Physiol 291:L58-L65), and bones (Jahr H, et al. (2005) Biochem Biophys Res Commun 337:349-354).

Within cutaneous primary sensory neurons where ASICs have been most characterized, evidence exists for various subunit expression profiles in different subpopulations of dorsal root ganglion (DRG) neurons. The majority of ASIC subunits are found in DRG neurons (ASIC1a-b, ASIC2a-b, and ASIC3) (Alvarez de la Rosa D, et al. (2002) Proc Natl Acad Sci USA 99:2326-2331). Histochemical analysis shows the presence of ASIC2 and 3 in specialized cutaneous nerve endings (e.g., Meissner's corpuscules, palisades of lanceolate fibers, Pilo-Ruffini nerve endings, Merkel cells, small free nerve endings) (Price M P, et al. (2001) Neuron 32:1071-1083, 2001; Price, et al., 2000). There is some evidence of changes in ASIC subunit expression during inflammation where an up-regulation of ASIC3 is reported (Mamet J, et al. (2002) J Neurosci 22:10662-10670; Voilley N, et al. J Neurosci 21:8026-8033, 2001; Mamet, et al., 2002; McIlwrath S L, et al. (2005) Neuroscience 131:499-511) and with nerve injury where a down-regulation of ASIC1a is observed (Poirot O, et al. (2006) J Physiol 576:215-234). Increases in Glial cell line-derived neurotrophic factor in the skin was shown to alter mechanical sensitivity of cutaneous nociceptors that correlates with an increase in the expression of ASIC2a and b subunits (Albers K M, et al. (2006) J Neurosci 26:2981-2990). Interestingly, ASIC2 has been implicated in mechanosensation (Price, et al., 2000).

It has long been understood that cardiac pain is associated with myocardial ischemia, typically described as discomfort or pain in the chest accompanied with a sense of strangling and anxiety—angina pectoris. Experimentally, occlusion of a coronary artery results in the activation of cardiac afferent in the sympathetic (Brown A M (1967) J Physiol 190:35-53) and parasympathetic pathways, which mediate powerful and opposing reflexes that contribute to cardiovascular homeostasis. Depending upon the location and extent of the ischemia, the vagal afferent can evoke hypotension, bradycardia, nausea and vomiting, while the ischemia-sensitive sympathetic afferents can induce hypertension, tachycardia, and the pain of angina pectoris. Activation of the cardiac sensory afferents has been attributed to the accumulation of several substances released during ischemia, e.g., ATP, 5HT, bradykinin, adenosine (Huang M H, et al. (1996) Cardiovasc Res 32:503-515; Euchner-Wamser I, et al. (1994) Pain 58:117-128; Armour J A, et al. (1994) Cardiovasc Res 28:1218-1225; James T N (1989) Anesth Analg 69:633-646). However, the precise mechanisms underlying cardiac pain during myocardial ischemia remain poorly understood.

Myocardial ischemia is also accompanied with a drop in intracellular and extracellular pH resulting from the high metabolic activity of the heart. The acidification of extracellular milieu can directly stimulate cardiac afferent sympathetic fibers. The role of ASICs in cardiac pain has been closely examined as these channels are expressed in cardiac sympathetic nociceptive neurons (Benson, et al., 1999). The large majority of those cardiac sensory neurons respond to mild sustained acidification (pH 7.0-pH 6.0). Additional studies indicate that ASIC3 is preferentially expressed in these neurons. Interestingly, the sensitivity of cardiac AS1C to extracellular pH could be positively modulated by the presence of lactic acid (lactic acid is produced by anaerobic metabolism during cardiac ischemia) (Immke D C et al. (2001) Nat Neurosci 4:869-870). Finally, while ASIC responses were highly represented in cardiac sympathetic neurons (93% neurons studied), a significant portion of parasympathetic neurons (74%), residing within the nodose ganglion, also displayed AS1C-like responses, albeit smaller both in amplitude and frequency (Benson, et al., 1999). Whether the expression level of ASICs can be modulated in angina pectoris or other pathologies of the heart is not known.

Visceral mechanoreceptors are critical for the perception of sensation and autonomic reflex control of gastrointestinal function. Not unlike for cutaneous mechanosensation, ASICs have now been recognized to play a significant role in visceral mechanosensation. The table below shows the relative contribution of the different ASIC subtypes in GI motility and mechanosensation (Page, et al., 2005b; Page, et al., 2004; Page A J, et al. (2007) Pain. 133 (1-3):150-60. Epub 2007 Apr. 27).

| | | | Gastric | | | Fecal |
|---|---|---|---|---|---|---|
| | Gastro-oesophageal | | empty- | Colonic | | pellet |
| | Mucosal | Tension | ing | Serosal | Mesenteric | Output |
| ASIC1 | ↑ | ↑ | ↓ | ↑ | ↑ | [symbol] |
| ASIC2 | ↑ | ↓↓ | [symbol] | ↑↑ | [symbol] | ↓ |
| ASIC3 | [symbol] | ↓↓ | [symbol] | ↓↓ | ↓↓ | [symbol] |

Modulation of ASIC expression in the GI track has been shown with ASIC 3 both in human and in animals. ASIC3 is up-regulated in Crohn's disease (Yiangou Y, et al. (2001) Eur J Gastroenterol Hepatol 13:891-896). Similarly in mice, sensitization of colonic afferents by inflammatory mediators involves ASIC3 (Jones R C, III, et al. (2005) J Neurosci 25:10981-10989).

Abnormally high osteoclastic bone resorption is a hallmark of several painful bone pathologies such as metastatic bone disease, Paget's disease of bones, osteoporosis, fibrous dysplasia, osteogenesis imperfecta, or bone metastases (Adami S, et al. (2002) Clin Exp Rheumatol 20:55-58; Astrom E et al. (1998) Acta Paediatr 87:64-68; Fulfaro F, et al. (1998) Pain 78:157-169; Gangji V et al. (1999) Clin Rheumatol 18:266-267; Lane J M, et al. (2001) Clin Orthop Relat Res 6-12; Mantyh P W (2002) Pain 96:1-2). Inhibition of osteoclastic bone resorption (e.g., with bisphosphonates, calcitonin, mithramycin, gallium nitrate) is often effective at reducing pain associated with these conditions. The mechanism of bone resorption is dependent upon the secretion of protons via the action of the vacuolar H⁺-ATPase pump causing local tissue acidosis. Tissue acidosis can trigger pain signaling in many tissues through the activation of the ASICs and/or TRPV1 and sensory fibers that innervate bones express both of these channels (Mach D B, et al. (2002) Neuroscience 113:155-166). Furthermore, both human osteoblasts and osteoclasts express several subtypes of ASICs (1, 2, 3, and 4) (Jahr, et al., 2005); it is speculated that these could be thought to be involved in the modulation of bone function by pH.

In bone cancer, acidosis-dependent bone resorption is thought to facilitate tumor colonization (Nagae M, et al. (2007) J Bone Miner Metab 25:99-104). DRG neurons that innervate the region where the tumor is present in the bone, show marked increased in the levels of expression of ASICs, specifically ASIC1a and 1b, but not ASIC3 or TRPV1.

The lungs are one of the major organs that are involved in pH homeostasis. As for other tissues, tissue acidosis in the pulmonary interstitium is observed when the production of CO₂ exceeds its elimination causing an accumulation or during anaerobic metabolism that results in the accumulation of lactic acid during tissue ischemia or hypoxia. This can occur under physiological conditions (e.g., exercise) and pathological conditions (e.g., chronic obstructive pulmonary diseases) (Berger K I, et al. (2000) J Appl Physiol 88:257-264). A possible mechanism by which lungs can detect and respond to changes in pH homeostasis is through the activation of ASICs. For example, several types of ASIC currents are found in vagal pulmonary primary sensory neurons (Gu et al., 2006). Activation of these afferents (c-fibers) causes bronchoconstriction, mucus hypersecretion, cough, dyspneic sensation and bronchial vasodilatation. Furthermore, ASIC3 and TRPV1 are expressed in spinal DRG neurons that innervate the rat lung and pleura (Groth M, et al. (2006) Respir Res 7:96) where they could play a role in pleural pain sensation.

The expression of ASIC in the lung may not be limited to the innervation system. Lung epithelium has been reported to express ASIC3 (Su X F, et al. (2006) Inter-regulation of proton-gatede Na+ channel 3 and cystic fibrosis transmembrane conductance regulator. J Biol Chem). Furthermore, a direct functional interaction between ASIC3 and the Cystic Fibrosis Transmembrane conductance Regulator (CFTR) has been demonstrated. Since there is significant acidification of the luminal fluid in the airways of cystic fibrosis patients that could lead to the activation of ASIC3, this interaction could contribute to the defective transepithelial transport of salt and fluids seen in these patients.

Transcripts for ASIC1a and ASIC2a, b are widely expressed in the CNS but there is little or no ASIC3 or ASIC1b (Garcia-Anoveros J, et al. (1997) Proc Natl Acad Sci USA 94:1459-1464; Price M P, et al. (1996) J Biol Chem 271:7879-7882). In rodents, ASIC1a is enriched in glomerulus of the olfactory bulb, wisker barrel cortex, cingular cortex, striatum, nucleus accumbens, amygdala, and cerebral cortex (Wemmie J A, et al. (2003) J Neurosci 23:5496-5502). The distribution of ASIC2 is less well defined but appears enriched in the cerebellum (Jovov B et al. (2003) Histochem Cell Biol 119:437-446). ASIC1 appears to be closely associated with postsynaptic structures of dendritic spines (colocalized with PSD-95) at glutamatergic synapses, although histochemical data shows a broad neuronal distribution (cell body, dendrites and axons).

ASIC1 has been implicated as a player in stroke and brain ischemia (Xiong Z G, et al. (2004) Cell 118:687-698, 2004; Xiong Z G, et al. (2006) J Membr Biol 209:59-68; Mach, et al., 2002). Extracellular pH during brain ischemia can decrease substantially (pH 6.3 and below). Since ASIC1a are calcium permeable, this channel may contribute to the Ca⁺⁺ overload and neuronal cell death observed with strokes. Blockage of ASIC1a has been shown to be neuroprotective during brain ischemia (Xiong, et al., 2004) and thus, it is speculated that altered expression of ASIC1a could lead to increased sensitivity to stroke. Furthermore, ASIC1a may contribute to neuronal degeneration associated with autoimmune diseases such as multiple sclerosis (Friese et al. (2007) Nat. Med. 13 (12): 1483-1489).

ASIC1a is highly enriched in the amygdala and other brain region involved in anxiety. Loss of ASIC1a causes a pronounced reduction in fear-related behavior. This would translate to risk-taking behavior in humans (Poulton R et al. (2002) Behav Res Ther 40:127-149). Conversely, ASIC1a over-expression produces increase fear-related behavior. In humans, patients with panic disorders have increased anxiety and panic attacks when breathing CO₂ enriched air (Klein D F (1993) Arch Gen Psychiatry 50:306-317). CO₂ is known to lower brain pH. (All of the aforementioned references are incorporated herein in their entirety).

Based on the above, there is a need for compositions that modulate the activity of ion channels and methods of use thereof for the treatment of conditions, diseases and disorders related to pain, inflammation, the neurological system, the gastrointestinal system and genitourinary system. There is also a need for ion channel-targeting imaging agents for the detection and/or diagnosis of conditions, diseases and disorders related to pain, inflammation, the neurological system, the gastrointestinal system and genitourinary system, as well as cancer.

### DEFINITIONS

“ASIC imaging agents,” and “ion channel-targeting agents” include radiolabelled ion channel targeting molecules, e.g., the compounds of Formulae I and II, for imaging areas of ion channel (e.g., ASIC) expression, e.g., in vivo, and/or for the treatment of ion channel-related conditions in a patient, such as those described herein.

“Ion channel-targeting agents” include labeled molecules such as those described herein that, without being bound by theory, associate or bind (i.e., “target”) with ion channels (e.g., ASIC).

“Imaging agent,” “contrast agent,” “ion channel-targeting agents” or “ASIC imaging agents,” terms that may be used interchangeably, refer to any agent that may be used in connection with methods for imaging an internal region of a patient and/or diagnosing the presence or absence of a disease in a patient by the application and/or detection of an energy source. Exemplary imaging agents include contrast agents for use in connection with ultrasound, magnetic resonance imaging, radionuclide imaging, or x-ray (including computed tomography) imaging of a patient, and the compositions described herein.

The language “ion channel-related condition,” as used herein, describes diseases and disorders that may be treated or prevented (or a symptom of such disease or disorder that may be reduced) by the compounds of the invention. In one embodiment, an ion channel-related condition is associated with pain, inflammation, cardiovascular disorders, respiratory conditions, genitourinary disorders, gastrointestinal disorders, cancers and neurological disorders. In another particular embodiment, the ion channel-related condition is associated with bone cancer.

As used herein, the term “acid” refers to carboxylic acid, sulfonic acid, sulfinic acid, sulfamic acid, phosphonic acid and boronic acid functional groups.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) includes both “unsubstituted alkyl” and “substituted alkyl”, the latter of which refers to alkyl moieties having substituents replacing a hydrogen on one or more carbons of the hydrocarbon backbone, which allow the molecule to perform its intended function. The term “substituted” is intended to describe moieties having substituents replacing a hydrogen on one or more atoms, e.g. C, O or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, morpholino, phenol, benzyl, phenyl, piperizine, cyclopentane, cyclohexane, pyridine, 5H-tetrazole, triazole, piperidine, or an aromatic or heteroaromatic moiety.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, thiol, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) can be further derivatized with an oxime moiety, e.g., an aldehyde moiety can be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “amine” or “amino” should be understood as being broadly applied to both a molecule, or a moiety or functional group, as generally understood in the art, and can be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

In a particular embodiment of the invention, the term “amine” or “amino” refers to substituents of the formulas N(R⁸)R⁹ or C₁₋₆—N(R⁸)R⁹, wherein R⁸ and R⁹ are each, independently, selected from the group consisting of —H and —(C₁₋₄alkyl)₀₋₁G, wherein G is selected from the group consisting of —COOH, —H, —PO₃H, —SO₃H, —Br, —Cl, —F, —O—C₁₋₄alkyl, —S—C₁₋₄alkyl, aryl, —C(O)OC₁-C₆-alkyl, —C(O)C₁₋₄alkyl-COOH, —C(O)C₁-C₄-alkyl and —C(O)-aryl; or N(R⁸)R⁹ is pyrrolyl, tetrazolyl, pyrrolidinyl, pyrrolidinyl-2-one, dimethylpyrrolyl, imidazolyl and morpholino.

The term “aryl” includes groups, including 5- and 6-membered single-ring aromatic groups that can include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure can also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

It will be noted that the structures of some of the compounds of this invention include asymmetric carbon atoms. It is to be understood accordingly that the isomers arising from such asymmetry (e.g., all enantiomers and diastereomers) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein can be obtained through art recognized synthesis strategies.

As used herein, the terms “gated ion channel,” “gated channel” or “ion channel” are used interchangeably and are intended to refer to a mammalian (e.g., rat, mouse, human) multimeric complex responsive to, for example, variations of voltage (e.g., membrane depolarization or hyperpolarization), temperature (e.g., higher or lower than 37° C.), pH (e.g., pH values higher or lower than 7.4), ligand concentration and/or mechanical stimulation. Examples of specific modulators include, but are not limited to, endogenous extracellular ligands such as anandamide, ATP, glutamate, cysteine, glycine, gamma-aminobutyric acid (GABA), histamine, adenosine, serotonin (5HT), acetylcholine, epinephrine, norepinephrine, protons, ions, e.g., Na⁺, Ca⁺⁺, K⁺, Cl⁻, H⁺, Zn⁺, and/or peptides, e.g., Met-enkephaline, Leu-enkephaline, dynorphin, neurotrophins, and/or the RFamide related peptides, e.g., FMRFamide and/or FLRFamide; to endogenous intracellular ligands such as cyclic nucleotides (e.g. cyclicAMP, cyclicGMP), Ca⁺⁺ and/or G-proteins; to exogenous extracellular ligands or modulators such as α-amino-3-hydroxy-5-methyl-4-isolaxone propionate (AMPA), amiloride, capsaicin, capsazepine, epibatidine, cadmium, barium, gadolinium, guanidium, kainate, N-methyl-D-aspartate (NMDA). Gated ion channels also include complexes responsive to toxins, examples of which include, but are not limited to, Agatoxin (e.g. α-agatoxin IVA, IVB, ω-agatoxin IVA, TK), Agitoxins (Agitoxin 2), Apamin, Argiotoxins, Batrachotoxins, Brevetoxins (e.g. Brevetoxin PbTx-2, PbTx-3, PbTx-9), Charybdotoxins, Chlorotoxins, Ciguatoxins, Conotoxins (e.g. α-conotoxin GI, GIA, GII, IMI, MI, MII, SI, SIA, SII, and/or EI; δ-conotoxins, α-conotoxin GIIIA, GIIIB, GIIIC and/or GS, ω-conotoxin GVIA, MVIIA MVIIC, MVIID, SVIA and/or SVIB), Dendrotoxins, Grammotoxins (GsMTx-4, ω-grammotoxin SIA), Grayanotoxins, Hanatoxins, Iberiotoxins, Imperatoxins, Jorotoxins, Kaliotoxins, Kurtoxins, Leiurotoxin 1, Pricotoxins, Psalmotoxins, (e.g., Psalmotoxin 1 (PcTx1)), Margatoxins, Noxiustoxins, Phrixotoxins, PLTX II, Saxitoxins, Stichodactyla Toxins, sea anemone toxins (e.g. APETx2 from Anthopleura elegantissima), Tetrodotoxins, Tityus toxin K-α, Scyllatoxins and/or tubocurarine.

In a preferred embodiment, the compounds of the invention modulate the activity of ASIC1a and/or ASIC3.

“Gated ion channel-mediated activity” is a biological activity that is normally modulated (e.g., inhibited or promoted), either directly or indirectly, in the presence of a gated ion channel. Gated ion channel-mediated activities include, for example, receiving, integrating, transducing, conducting, and transmitting signals in a cell, e.g., a neuronal or muscle cell. A biological activity that is mediated by a particular gated ion channel, e.g. ASIC1a or ASIC3, is referred to herein by reference to that gated ion channel, e.g. ASIC1a- or ASIC3-mediated activity. To determine the ability of a compound to inhibit a gated ion channel-mediated activity, conventional in vitro and in vivo assays can be used which are described herein.

“Neurotransmission,” as used herein, is a process by which small signaling molecules, termed neurotransmitters, are rapidly passed in a regulated fashion from a neuron to another cell. Typically, following depolarization associated with an incoming action potential, a neurotransmitter is secreted from the presynaptic neuronal terminal. The neurotransmitter then diffuses across the synaptic cleft to act on specific receptors on the postsynaptic cell, which is most often a neuron but can also be another cell type (such as muscle fibers at the neuromuscular junction). The action of neurotransmitters can either be excitatory, depolarizing the postsynaptic cell, or inhibitory, resulting in hyperpolarization. Neurotransmission can be rapidly increased or decreased by neuromodulators, which typically act either pre-synaptically or post-synaptically. The gated ion channel ASIC1a has been shown to possibly contribute to neurotransmission (Babini et al., J Biol. Chem. 277 (44):41597-603 (2002); Gao et al. (2005) Neuron 48: 635-646).

Examples of gated ion channel-mediated activities include, but are not limited to, pain (e.g., inflammatory pain, acute pain, chronic malignant pain, chronic nonmalignant pain and neuropathic pain), inflammatory disorders, diseases and disorders of the genitourinary and gastrointestinal systems, and neurological disorders (e.g., neurodegenerative or neuropsychiatric disorders).

“Pain” is defined as an unpleasant sensory and emotional experience associated with actual or potential tissue damage, or described in terms of such damage (International Association for the Study of Pain—IASP). Pain is classified most often based on duration (i.e., acute vs. chronic pain) and the underlying pathophysiology (i.e., nociceptive vs. neuropathic pain).

Acute pain can be described as an unpleasant experience with emotional and cognitive, as well as sensory, features that occur in response to tissue trauma and disease and serves as a defensive mechanism. Acute pain is usually accompanied by a pathology (e.g., trauma, surgery, labor, medical procedures, acute disease states) and the pain resolves with healing of the underlying injury. Acute pain is mainly nociceptive, but can also be neuropathic.

Chronic pain is pain that extends beyond the period of healing, with levels of identified pathology that often are low and insufficient to explain the presence, intensity and/or extent of the pain (American Pain Society—APS). Unlike acute pain, chronic pain serves no adaptive purpose. Chronic pain can be nociceptive, neuropathic, or both and caused by injury (e.g., trauma or surgery), malignant conditions, or a variety of chronic conditions (e.g., arthritis, fibromyalgia and neuropathy). In some cases, chronic pain exists de novo with no apparent cause.

“Nociceptive pain” is pain that results from damage to tissues and organs. Nociceptive pain is caused by the ongoing activation of pain receptors in either the superficial or deep tissues of the body. Nociceptive pain is further characterized as “somatic pain”, including “cutaneous pain” and “deep somatic pain”, and “visceral pain”.

“Somatic pain” includes “cutaneous pain” and “deep somatic pain.” Cutaneous pain is caused by injury, diseases and disorders of the skin and related organs. Examples of conditions associated with cutaneous pain include, but are not limited to, cuts, burns, infections, lacerations, as well as traumatic injury and post-operative or surgical pain (e.g., at the site of incision).

“Deep somatic pain” results from injuries, diseases or disorders of the musculoskeletal tissues, including ligaments, tendons, bones, blood vessels and connective tissues. Examples of deep somatic pain or conditions associated with deep somatic pain include, but are not limited to, sprains, broken bones, arthralgia, vasculitis, myalgia and myofascial pain. Arthralgia refers to pain caused by a joint that has been injured (such as a contusion, break or dislocation) and/or inflamed (e.g., arthritis). Vaculitis refers to inflammation of blood vessels with pain. Myalgia refers to pain originating from the muscles. Myofascial pain refers to pain stemming from injury or inflammation of the fascia and/or muscles.

“Visceral” pain is associated with injury, inflammation or disease of the body organs and internal cavities, including but not limited to, the circulatory system, respiratory system, gastrointestinal system, genitourinary system, immune system, as well as ear, nose and throat. Visceral pain can also be associated with infectious and parasitic diseases that affect the body organs and tissues. Visceral pain is extremely difficult to localize, and several injuries to visceral tissue exhibit “referred” pain, where the sensation is localized to an area completely unrelated to the site of injury. For example, myocardial ischaemia (the loss of blood flow to a part of the heart muscle tissue) is possibly the best known example of referred pain; the sensation can occur in the upper chest as a restricted feeling, or as an ache in the left shoulder, arm or even hand. Phantom limb pain is the sensation of pain from a limb that one no longer has or no longer gets physical signals from—an experience almost universally reported by amputees and quadriplegics.

“Neuropathic pain” or “neurogenic pain” is pain initiated or caused by a primary lesion, dysfunction or perturbation in the nervous system. “Neuropathic pain” can occur as a result of trauma, inflammation or disease of the peripheral nervous system (“peripheral neuropathic pain”) and the central nervous system (“central pain”). For example, neuropathic pain can be caused by a nerve or nerves that are irritated, trapped, pinched, severed or inflamed (neuritis). There are many neuropathic pain syndromes, such as diabetic neuropathy, trigeminal neuralgia, postherapeutic neuralgia (“shingles”), post-stroke pain, and complex regional pain syndromes (also called reflex sympathetic dystrophy or “RSD” and causalgia).

As used herein, the term “inflammatory disease or disorder” includes diseases or disorders which are caused, at least in part, or exacerbated by, inflammation, which is generally characterized by increased blood flow, edema, activation of immune cells (e.g., proliferation, cytokine production, or enhanced phagocytosis), heat, redness, swelling, pain and loss of function in the affected tissue and organ. The cause of inflammation can be due to physical damage, chemical substances, micro-organisms, tissue necrosis, cancer or other agents. Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some disorders can fall within one or more categories.

The terms “neurological disorder” and “neurodegenerative disorder” refer to injuries, diseases and dysfunctions of the nervous system, including the peripheral nervous system and central nervous system. Neurological disorders and neurodegenerative disorders include, but are not limited to, diseases and disorders that are associated with gated ion channel-mediated biological activity. Examples of neurological disorders include, but are not limited to, Alzheimer's disease, epilepsy, cancer, neuromuscular diseases, multiple sclerosis, amyotrophic lateral sclerosis, stroke, cerebral ischemia, neuropathy (e.g., chemotherapy-induced neuropathy, diabetic neuropathy), retinal pigment degeneration, Huntington's chorea, and Parkinson's disease, anxiety disorders (e.g., phobic disorders (e.g., agoraphobia, claustrophobia), panic disorders, phobias, anxiety hysteria, generalized anxiety disorder, and neurosis), and ataxia-telangiectasia.

As used herein, “neuropathy” is defined as a failure of the nerves that carry information to and from the brain and spinal cord resulting in one or more of pain, loss of sensation, and inability to control muscles. In some cases, the failure of nerves that control blood vessels, intestines, and other organs results in abnormal blood pressure, digestion problems, and loss of other basic body processes. Peripheral neuropathy can involve damage to a single nerve or nerve group (mononeuropathy) or can affect multiple nerves (polyneuropathy).

The term “treated,” “treating” or “treatment” includes the diminishment or alleviation of at least one symptom associated with the pain, inflammatory disorder, neurological disorder, genitourinary disorder or gastrointestinal disorder (e.g., a symptom associated with or caused by gated ion channel mediated activity) being treated. In certain embodiments, the treatment comprises the modulation of the interaction of a gated ion channel (e.g., ASIC1a and/or ASIC3) by a gated ion channel modulating compound, which would in turn diminish or alleviate at least one symptom associated with or caused by the gated ion channel-mediated activity being treated. For example, treatment can be diminishment of one or several symptoms of a disorder or complete eradication of a disorder.

As used herein, the phrase “therapeutically effective amount” of the compound is the amount necessary or sufficient to treat or prevent pain, an inflammatory disorder, a neurological disorder, a gastrointestinal disorder or a genitourinary disorder, (e.g., to prevent the various symptoms of a gated ion channel-mediated activity). In an example, an effective amount of the compound is the amount sufficient to alleviate at least one symptom of the disorder, e.g., pain, inflammation, a neurological disorder, a gastrointestinal disorder or a genitourinary disorder, in a subject.

The term “subject” is intended to include animals, which are capable of suffering from or afflicted with a gated ion channel-associated state or gated ion channel-associated disorder, or any disorder involving, directly or indirectly, gated ion channel activity. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from pain, inflammation, a neurological disorder, a gastrointestinal disorder or a genitourinary disorder (e.g. associated with gated channel-associated activity).

The language “gated ion channel modulator” or “modulator of gated ion channel activity” refers to compounds that modulate, i.e., inhibit, promote or otherwise alter the activity of a gated ion channel. For example, the gated ion channel modulator can inhibit, promote or otherwise alter the response of a gated ion channel to, for example, variations of voltage (e.g., membrane depolarization or hyperpolarization), temperature (e.g., higher or lower than 37° C.), pH (e.g., pH values higher or lower than 7.4), ligand concentration and/or mechanical stimulation. Examples of gated ion channel modulators include compounds of the invention (i.e., the compounds of Formulae I, II and III, as well as the species described herein) including salts thereof, e.g., a pharmaceutically acceptable salt. In a particular embodiment, the gated ion channel modulators of the invention can be used to treat a disease or disorder associated with pain, inflammation, neurological disorders, gastrointestinal disorders or genitourinary disorders in a subject in need thereof. In another embodiment, the compounds of the invention can be used to treat an inflammatory disorder in a subject in need thereof.

### Modulators of Ion Channel Activity

The present invention provides compounds that modulate the activity of a gated ion channel in a patient. The present invention also provides compounds that can be used to diagnose a gated ion-channel related condition in a patient. In one embodiment, the compounds of the invention diagnose a gated ion-channel related condition in a patent by targeting one or more gated ion-channels. In some embodiments, the compounds of the invention modulate the activity of or target a gated ion channel comprised of at least one subunit belonging to the DEG/ENaC, TRP and/or P2X gene superfamilies. In some embodiments, the compounds of the invention modulate the activity of or target a gated ion channel comprised of at least one subunit selected from the group consisting of αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6, TRPA1 and TRPM8. In still other embodiments, the compounds of the invention modulate the activity of or target a DEG/ENaC gated ion channel comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4. In certain embodiments, the compounds of the invention modulate the activity of or target a DEG/ENaC gated ion channel comprised of at least one subunit selected from the group consisting of ASIC1a, ASIC1b, ASIC2a, ASIC2b, ASIC3, and ASIC4. In certain embodiments, the compounds of the invention modulate the activity of or target a DEG/ENaC gated ion channel comprised of at least two subunits selected from the group consisting of ASIC1a, ASIC1b, ASIC2a, ASIC2b, ASIC3, and ASIC4. In yet other embodiments, the compounds of the invention modulate the activity of or target a DEG/ENaC gated ion channel comprised of at least three subunits selected from the group consisting of ASIC1a, ASIC1b, ASIC2a, ASIC2b, ASIC3, and ASIC4. In certain embodiments, the compounds of the invention modulate the activity of or target a gated ion channel comprised of ASIC, i.e., ASIC1a or ASIC1b. In certain embodiments, the compounds of the invention modulate the activity of or target a gated ion channel comprised of ASIC3. In certain embodiments, the compounds of the invention modulate the activity of or target a gated ion channel comprised of ASIC1a and ASIC2a; ASIC1a and ASIC3; ASIC1b and ASIC3; ASIC2a and ASIC2b; ASIC2a and ASIC3; ASIC2b and ASIC3; and ASIC1a, ASIC2a and ASIC3. In other embodiments, the compounds of the invention modulate the activity of or target the P2X gated ion channel comprised of at least one subunit selected from the group consisting of P2X₁, P2X₂, P2X₃, P2X₄, P2X₅, P2X₆, and P2X₇. In certain embodiments, the compounds of the invention modulate the activity of or target a gated ion channel comprised of P2X₂, P2X₃ or P2. In certain embodiments, the compounds of the invention modulate the activity of or target a gated ion channel comprised of P2X₁ and P2X₂, P2X₁ and P2X₅, P2X₂ and P2X₃, P2X₂ and P2X₆, and P2X₄ and P2X₆. In yet another aspect of the invention, the compounds of the invention modulate the activity of or target a TRP gated ion channel comprised of at least one subunit selected from the group TRPV1, TRPV2, TRPV3, TRPV4, TRPV5, TRPV6, TRPA1 and TRPM8. In certain embodiments, the compounds of the invention modulate the activity of or target a gated ion channel comprised of TRPV1 or TRPV2. In certain embodiments, the compounds of the invention modulate the activity of or target a gated ion channel comprised of TRPV1 and TRPV2, TRPV1 and TRPV4, and TRPV5 and TRPV6.

In a particular embodiment, the compounds of the invention modulate the activity of or target ASIC1a and/or ASIC3.

In one aspect, the invention provides an ASIC imaging agent of the Formula I:

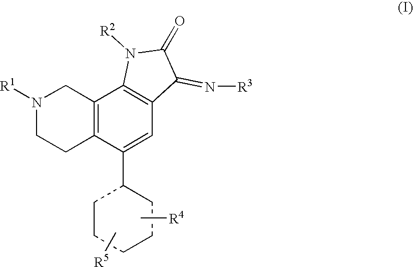

and pharmaceutically acceptable salts, enantiomers, stereoisomers, rotamers, tautomers, diastereomers, or racemates thereof;

the dashed lines indicate a single or double bond;

R¹ is selected from the group consisting of hydrogen, alkyl, alkoxy-alkyl, hydroxy-alkyl, alkoxy-carbonyl-alkyl, alkyl-carbonyl-oxy-alkyl, cycloalkyl, cycloalkyl-alkyl, alkenyl, alkynyl, alkoxy, sulfonamide, amino, sulfonyl, sulfonic acid, urea, phenyl or benzyl, in which the phenyl or benzyl group is optionally substituted with halogen, CF₃, nitro, amino, cyano, hydroxy-alkyl, alkoxy, sulfonamide, alkenyl, alkynyl, amino, sulfonyl, sulfonic acid and urea;

R² is selected from the group consisting of hydrogen, hydroxyl, alkyl, alkenyl, alkynyl, —(CH₂)₁₋₄S(O)₃H, —C(O)C₁₋₄alkyl and —S(O)₂C₁₋₄alkyl;

R³ is selected from the group consisting of hydrogen, hydroxyl, alkyl, acyl, phenyl, benzyl, —(CH₂)₁₋₄COOH, —C(O)N(CH₃)₂, —O-phenyl, —OCF₃, alkoxy, —O(CH₂)₀₋₄OCH₃, —C(O)H, —C(O)CH₃,

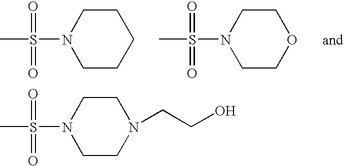

and R⁴ and R⁵ are each, independently, selected from the group consisting of hydrogen, halogen, CF₃, nitro, amino, cyano, hydroxyl, alkyl, alkoxy, phenoxy and phenyl, or a group of the formula —SO₂NR′R″, wherein R′ and R″ independently of each another represents hydrogen or alkyl;

wherein at least one of the atoms of Formula I is an isotope.

In one embodiment, the compound of Formula I is represented by the Formula II,

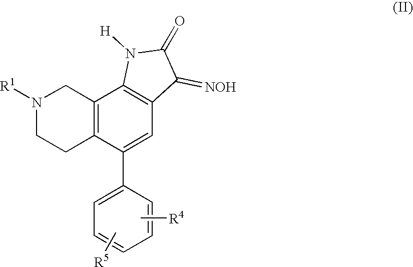

and pharmaceutically acceptable salts, enantiomers, stereoisomers, rotamers, tautomers, diastereomers, or racemates thereof;

wherein

R¹ is selected from the group consisting of hydrogen, alkyl, alkoxy-alkyl, alkoxy-carbonyl-alkyl, alkyl-carbonyl-oxy-alkyl, cycloalkyl, cycloalkyl-alkyl, alkenyl, alkynyl, alkoxy, sulfonamide, amino, sulfonyl, sulfonic acid, urea phenyl or benzyl, in which the phenyl or benzyl group is optionally substituted with halogen, CF₃, nitro, amino, cyano, hydroxy-alkyl, alkoxy, sulfonamide, alkenyl, alkynyl, amino, sulfonyl, sulfonic acid and urea; and

R⁴ and R⁵ are each, independently, selected from the group consisting of hydrogen, halogen, phenoxy, CF₃, nitro, amino, cyano, hydroxyl, alkyl, alkoxy and phenyl, or a group of the formula —SO₂NR′R″, wherein R′ and R″ independently of each another represents hydrogen or alkyl;

wherein at least one of the atoms of Formula II is an isotope.

In one embodiment of Formula II, R¹ is selected from the group consisting of hydrogen, C₁₋₄-alkyl, C₁₋₄-alkenyl, and C₁₋₄-alkynyl; and R⁴ and R⁵ are each, independently, selected from the group consisting of halogen, CF₃, nitro, amino, cyano, hydroxyl, C₁₋₄-alkyl, C₁₋₄-alkoxy, phenoxy and phenyl, wherein at least one of the atoms of Formula II is an isotope.

In one embodiment of the compounds of Formulae I or II, the isotope of the ASIC imaging agent is selected from the group consisting of ²H, ³H, ¹³C, ¹⁴C, ¹⁵N, ¹⁸O, ¹⁷O, ³¹P, ³²P, ³⁵S, ¹⁸F, ³⁶Cl, ¹²³I, ¹²⁵I, ¹²⁷I, ¹²⁹I, ¹³⁰I, and ¹³¹I. In another embodiment, the isotope is selected from the group consisting of ³H and ¹⁴C.

Certain exemplary compounds of the invention (i.e., compounds of the Formulae I and II) are listed below in Table 1, and are also referred to as “compounds of the invention.” The species listed include all pharmaceutically acceptable salts, polymorphs, enantiomers, stereoisomers, rotamers, tautomers, diastereomers, or racemates thereof.

TABLE 1

| | |
|---|---|
| 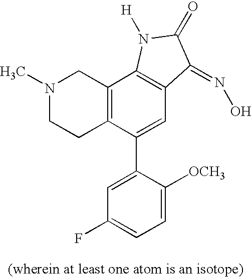 | 5-(5-fluoro-2-methoxyphenyl)-6,7,8,9- tetrahydro-3-(hydroxyimino)-8-methyl-1H- pyrrolo[3,2-h]isoquinoline-2(3H)-one (Compound A) |
| 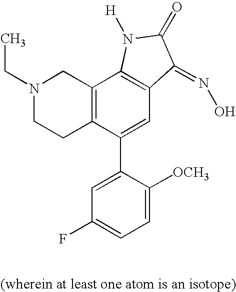 | 5-(5-fluoro-2-methoxyphenyl)-6,7,8,9- tetrahydro-3-(hydroxyimino)-8-ethyl-1H- pyrrolo[3,2-h]isoquinoline-2(3H)-one (Compound B) |
| 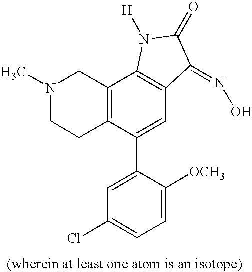 | 5-(5-chloro-2-methoxyphenyl)-6,7,8,9- tetrahydro-3-(hydroxyimino)-8-methyl-1H- pyrrolo[3,2-h]isoquinoline-2(3H)-one (Compound C) |
| 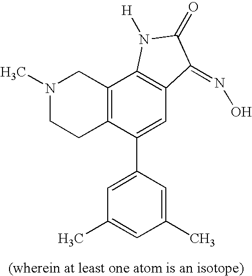 | 5-(3,5-dimethylphenyl)-6,7,8,9-tetrahydro- 3-(hydroxyimino)-8-methyl-1H- pyrrolo[3,2-h]isoquinoline-2(3H)-one (Compound D) |
| 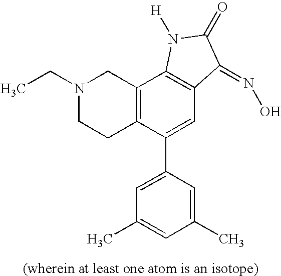 | 5-(3,5-dimethylphenyl)-6,7,8,9-tetrahydro- 3-(hydroxyimino)-8-ethyl-1H-pyrrolo[3,2- h]isoquinoline-2(3H)-one (Compound E) |
| 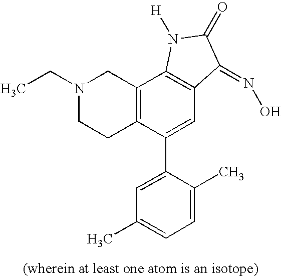 | 5-(2,5-dimethylphenyl)-6,7,8,9-tetrahydro- 3-(hydroxyimino)-8-ethyl-1H-pyrrolo[3,2- h]isoquinoline-2(3H)-one (Compound F) |
| 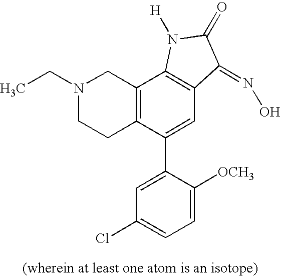 | 5-(5-chloro-2-methoxyphenyl)-6,7,8,9- tetrahydro-3-(hydroxyimino)-8-ethyl-1H- pyrrolo[3,2-h]isoquinoline-2(3H)-one (Compound G) |
| 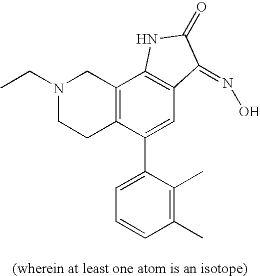 | 5-(2,3-dimethyl-phenyl)-6,7,8,9- tetrahydro-3-(hydroxyimino)-8-ethyl-1H- pyrrolo[3,2-h]isoquinoline-2(3H)-one (Compound I) |
| 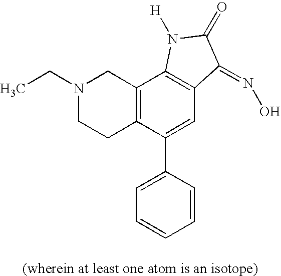 | 5-phenyl-6,7,8,9-tetrahydro-3- (hydroxyimino)-8-ethyl-1H-pyrrolo[3,2- h]isoquinoline-2(3H)-one (Compound H) |
| 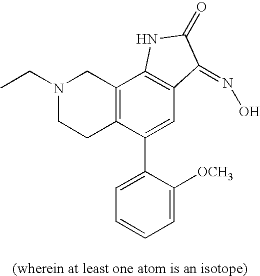 | 5-(2-methoxy-phenyl)-6,7,8,9-tetrahydro- 3-(hydroxyimino)-8-ethyl-1H-pyrrolo[3,2- h]isoquinoline-2(3H)-one (Compound J) |
| 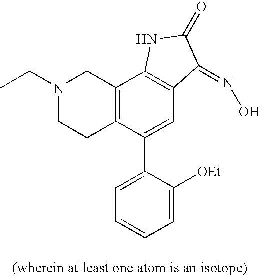 | 5-(2-ethoxy-phenyl)-6,7,8,9-tetrahydro-3- (hydroxyimino)-8-ethyl-1H-pyrrolo[3,2- h]isoquinoline-2(3H)-one (Compound K) |
| 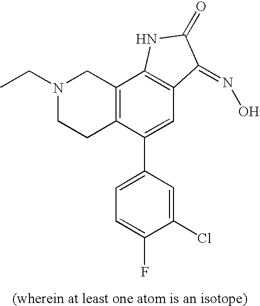 | 5-(3-chloro-4-fluoro-phenyl)-6,7,8,9- tetrahydro-3-(hydroxyimino)-8-ethyl-1H- pyrrolo[3,2-h]isoquinoline-2(3H)-one |
| 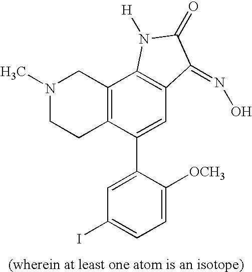 | 5-(5-iodo-2-methoxy-phenyl)-6,7,8,9- tetrahydro-3-(hydroxyimino)-8-methyl-1H- pyrrolo[3,2-h]isoquinoline-2(3H)-one |
| 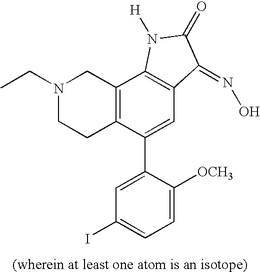 | 5-(5-iodo-2-methoxy-phenyl)-6,7,8,9- tetrahydro-3-(hydroxyimino)-8-ethyl-1H- pyrrolo[3,2-h]isoquinoline-2(3H)-one |
| 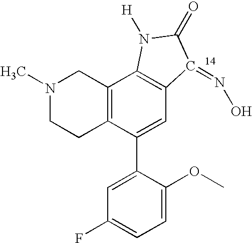 | |
| 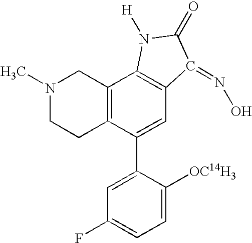 | |
| 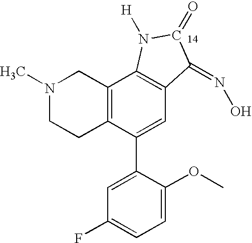 | |
| 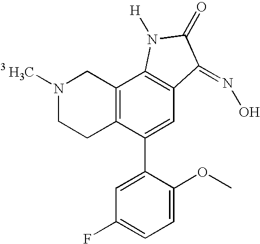 | |
| 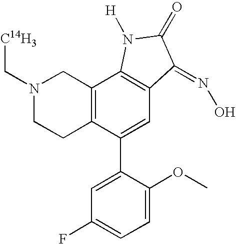 | |
| 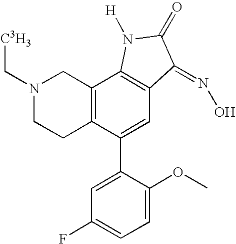 | |
| 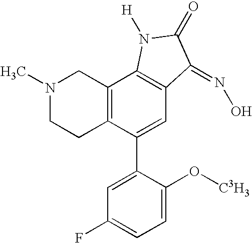 | |
| 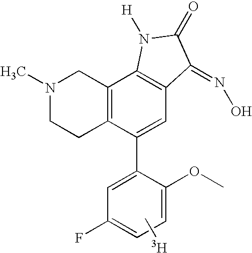 | |

The subject invention includes isotopically-labeled compounds of the Formulae I and II, meaning that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine, chlorine, and iodine such as ²H, ³H, ¹³C, ¹⁴C, ¹⁵N, ¹⁸O, ¹⁷O, ³¹P, ³²P, ³⁵S, ¹⁸F, ³⁶Cl, ¹²³I, ¹²⁵I, ¹²⁷I, ¹²⁹I, ¹³⁰I, and ¹³¹I respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances. Isotopically labeled compounds of Formulae I-II of this invention and prodrugs thereof can generally be prepared by carrying out the procedures exemplified below or those known in the art. For example, the isotopically-labeled compounds of the invention can be prepared by the methods outlined and exemplified in U.S. patent application Ser. No. 11/603,946 (incorporated herein by reference in its entirety) by substituting a readily available isotopically labeled reagent for a non-isotopically labeled reagent.

In the methods of treatment of the present invention, a metabolite of an ASIC antagonist can be used for the treatment of a gated-ion channel modulated disorder, e.g., pain. The metabolite can be administered to a subject directly, such as in a tablet, or the metabolite can be administered by being produced in the subject's body through metabolism. For example, a metabolite of the present invention can be effectively administered to a subject to treat a disease or condition by administering to the subject an amount of Compound A or Compound B, after which administration, the desired metabolite is formed in the subject's body through metabolism. Moreover, the administration route and dosage of Compound A and Compound B can be varied, as desired, to obtain desired in vivo concentrations and rates of production of a metabolite.

In one aspect, the compound that modulates the activity of a gated ion channel is a metabolite that is of the Formula III:

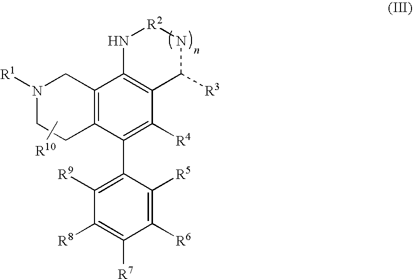

and pharmaceutically acceptable salts, enantiomers, stereoisomers, rotamers, tautomers, diastereomers, or racemates thereof;

wherein

R¹ is hydrogen or C₁-C₄ alkyl;

R² is O or C(O);

n is 0 or 1;

the dashed lines, independently, indicate a single or double bond;

R³ is ═NOH, —NO₂ or CO₂H;

R⁴, R⁵, R⁶, R⁷, R⁸ and R⁹ are each, independently, H, halogen, OH, CO₂H, C₁-C₄ alkyl, C₁-C₄ alkoxyl, hydroxy substituted C₁-C₄ alkyl, a C₁-C₄ aldehyde or a C₁-C₄ carboxylic acid; and

R¹⁰ is H, OH or ═O.

In exemplary embodiments of metabolites that are of the Formula III, R¹ is hydrogen, methyl or ethyl, R⁴ and R⁹ are each, independently, H or OH, R⁵ is H, CH₃, OCH₃, OCH₂CH₃, CH₂OH, OH, C(O)H or CO₂H, R⁶ is H, Cl, CH₃, CH₂OH, OH, C(O)H or CO₂H, R⁷ is H, F, OH or SO₂NMe₂, R⁸ is H, F, Cl, I, OH, CH₃, CH₂OH, C(O)H, CO₂H, and R¹⁰ is H.

In one embodiment, the compound of Formula III that modulates the activity of a gated ion channel is a metabolite of Compound A or Compound B that is of the Formula IV,

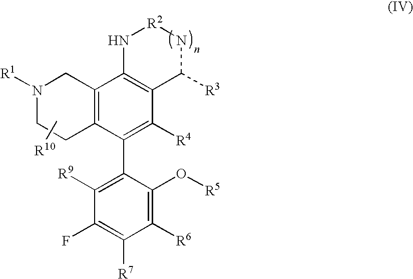

and pharmaceutically acceptable salts, enantiomers, stereoisomers, rotamers, tautomers, diastereomers, or racemates thereof;

wherein

R¹ is hydrogen, methyl or ethyl; R² is O or C(O); n is 0 or 1; the dashed lines, independently, indicate a single or double bond; R³ is ═NOH, —NO₂ or CO₂H; R⁵ is H or CH₃; R⁴, R⁶ and R⁷ are each, independently, selected from the group consisting of H and OH; R⁹ is H or OH; and R¹⁰ is H, OH or ═O. In one embodiment of Formula IV, R⁹ and R¹⁰ are H.

In one embodiment, the compound of Formula IV is represented by Compound L, which is a metabolite of Compound A:

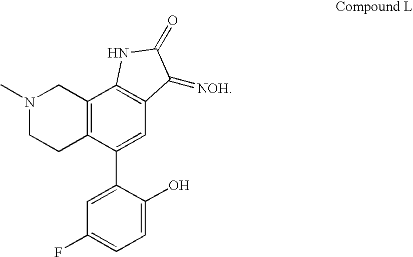

Certain exemplary compounds of the invention (i.e., compounds of the Formula III) are listed in Scheme A and Scheme B, and are also referred to as “compounds of the invention.” The species listed include all pharmaceutically acceptable salts, polymorphs, enantiomers, stereoisomers, rotamers, tautomers, diastereomers, or racemates thereof.

Other metabolites of Compound A and Compound B that are of the invention are listed in the exemplification section (see compounds of Schemes A-F) and are also referred to as “compounds of the invention.” The species listed include all pharmaceutically acceptable salts, polymorphs, enantiomers, stereoisomers, rotamers, tautomers, diastereomers, or racemates thereof.

It will be noted that the structures of some of the compounds of this invention include asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof. Compounds described herein can be obtained through art recognized synthesis strategies.

The description of the disclosure herein should be construed in congruity with the laws and principals of chemical bonding. For example, it may be necessary to remove a hydrogen atom in order to accommodate a substitutent at any given location.

Furthermore, it is to be understood that definitions of the variables (i.e., “R groups”), as well as the bond locations of the generic formulae of the invention, will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

In one embodiment of the invention, the compounds of the invention that modulate the activity of a gated ion channel are capable of chemically interacting with a gated ion channel, including αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6, TRPA1 and TRPM8. The language “chemical interaction” is intended to include, but is not limited to reversible interactions such as hydrophobic/hydrophilic, ionic (e.g., coulombic attraction/repulsion, ion-dipole, charge-transfer), covalent bonding, Van der Waals, and hydrogen bonding. In certain embodiments, the chemical interaction is a reversible Michael addition. In a specific embodiment, the Michael addition involves, at least in part, the formation of a covalent bond.

In a particular embodiment, the compounds of Formulae I, II, III and IV can be used to treat pain in a subject in need thereof. In one embodiment, the subject is a human.

In another embodiment, the compounds of Formulae I, II, III and IV can be used to treat inflammation in a subject in need thereof. In one embodiment, the subject is a human.

In a particular embodiment, Compound L can be used to treat pain in a subject in need thereof. In one embodiment, the subject is a human. In another embodiment, Compound L can be used to treat inflammation in a subject in need thereof. In one embodiment, the subject is a human.

Compounds of the inventions can be synthesized according to standard organic synthesis procedures that are known in the art. Representative synthesis procedures for compounds similar to the compounds of the invention can be found in U.S. application Ser. No. 11/603,946, which is incorporated herein by reference.

The end products of the reactions described herein can be isolated by conventional techniques, e.g. by extraction, crystallization, distillation, chromatography, etc.

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkyl-onium salts.

The chemical compound of the invention can be provided in dissoluble or indissoluble forms together with a pharmaceutically acceptable solvents such as water, ethanol, and the like. Dissoluble forms can also include hydrated forms such as the monohydrate, the dihydrate, the hemihydrate, the trihydrate, the tetrahydrate, and the like. In general, the dissoluble forms are considered equivalent to indissoluble forms for the purposes of this invention.

The chemical compounds of the present invention can exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallization of d- or l-(tartrates, mandelates, or camphorsulphonate) salts for example.

The chemical compounds of the present invention can also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, and Wilen S in “Enantiomers, Racemates, and Resolutions”, John Wiley and Sons, New York (1981).

Optical active compounds can also be prepared from optical active starting materials.

Moreover, some of the chemical compounds of the invention have an ═N—OH functional group, and thus exist in two forms, syn- and anti-form (Z- and E-form), depending on the arrangement of the substituents around the —C═N— double bond. A chemical compound of the present invention can thus be the syn- or the anti-form (Z- and E-form), or it can be a mixture hereof. It is to be understood that both the syn- and anti-form (Z- and E-form) of a particular compound is within the scope of the present invention, even when the compound is represented herein (i.e., through nomenclature or the actual drawing of the molecule) in one form or the other.

As such, in one embodiment, the E-isomers of the compounds of Formulae I or II can be used for the treatment of pain in subject in need thereof. In another embodiment, the Z-isomers of the compounds of Formulae I or II can be used for the treatment of pain in subject in need thereof. In another embodiment, the E-isomers of compound A or compound B can be used for the treatment of pain in subject in need thereof. In another embodiment, the Z-isomers of compound A or compound B can be used for the treatment of pain in subject in need thereof.

In yet another embodiment, the invention pertains to pharmaceutical compositions comprising gated ion channel modulating compounds described herein and a pharmaceutical acceptable carrier.

In another embodiment, the invention includes any novel compound or pharmaceutical compositions containing compounds of the invention described herein. For example, compounds and pharmaceutical compositions containing compounds set forth herein (e.g., compounds of the invention) are part of this invention, including salts thereof, e.g., pharmaceutically acceptable salts.

### Assays

The present invention relates to a method of modulating gated ion channel activity. As used herein, the various forms of the term “modulate” include stimulation (e.g., increasing or upregulating a particular response or activity) and inhibition (e.g., decreasing or downregulating a particular response or activity). In one aspect, the methods of the present invention comprise contacting a cell with an effective amount of a gated ion channel modulator compound, e.g. a compound of the invention, thereby modulating the activity of a gated ion channel. In certain embodiments, the effective amount of the compound of the invention inhibits the activity of the gated ion channel.

The gated ion channels of the present invention are comprised of at least one subunit belonging to the DEG/ENaC, TRP and/or P2X gene superfamilies. In one aspect the gated ion channel is comprised of at least one subunit selected from the group consisting of αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6, TRPA1 and TRPM8. In one aspect, the DEG/ENaC gated ion channel is comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4. In certain embodiments, the DEG/ENaC gated ion channel is comprised of at least one subunit selected from the group consisting of ASIC1a, ASIC1b, ASIC2a, ASIC2b, ASIC3, and ASIC4. In certain embodiments, the gated ion channel is comprised of ASIC1a, ASIC1b, or ASIC3. In another aspect of the invention, P2X gated ion channel is comprised of at least one subunit selected from the group consisting of P2X₁, P2X₂, P2X₃, P2, P2X₅, P2X₆, and P2X₇. In yet another aspect of the invention, the TRP gated ion channel is comprised of at least one subunit selected from the group TRPV1, TRPV2, TRPV3, TRPV4, TRPV5, TRPV6, TRPA1 and TRPM8. In another aspect, the gated ion channel is a heteromultimeric gated ion channel, including, but not limited to, αENaC, βENaC and γENaC; αENaC, βENaC and δENaC; ASIC1a and ASIC2a; ASIC1a and ASIC2b; ASIC1a and ASIC3; ASIC1b and ASIC3; ASIC2a and ASIC2b; ASIC2a and ASIC3; ASIC2b and ASIC3; ASIC1a, ASIC2a and ASIC3; ASIC3 and P2X, e.g. P2X₁, P2X₂, P2X₃, P2X₄, P2X₅, P2X₆ and P2X₇, preferably ASIC3 and P2X₂; ASIC3 and P2X₃; and ASIC3, P2X₂ and P2X₃; ASIC4 and at least one of ASIC1a, ASIC1b, ASIC2a, ASIC2b, and ASIC3; BLINaC (or hINaC) and at least one of ASIC1a, ASIC1b, ASIC2a, ASIC2b, ASIC3, and ASIC4; δENaC and ASIC, e.g. ASIC1a, ASIC1b, ASIC2a, ASIC2b, ASIC3 and ASIC4; P2X₁ and P2X₂, P2X₁ and P2X₅, P2X₂ and P2X₃, P2X₂ and P2X₆, P2X₄ and P2X₆, TRPV1 and TRPV2, TRPV5 and TRPV6, TRPV1 and TRPV4.

Assays for determining the ability of a compound within the scope of the invention to modulate the activity of gated ion channels are well known in the art and described herein in the Examples section. Other assays for determining the ability of a compound to modulate the activity of a gated ion channel are also readily available to the skilled artisan.

The gated ion channel modulating compounds of the invention can be identified using the following screening method, which method comprises the subsequent steps of

(i) subjecting a gated ion channel containing cell to the action of a selective activator, e.g., protons by adjustment of the pH to an acidic level, ATP by diluting sufficient amounts of ATP in the perfusion buffer or temperature by heating the perfusion buffer to temperatures above 37° C.;

(ii) subjecting a gated ion channel containing cell to the action of the chemical compound (the compound can be co-applied, pre-applied or post-applied); and

(iii) monitoring the change in membrane potential or ionic current induced by the activator, e.g., protons, on the gated ion channel containing cell. Alternatively, fluorescent imaging can be utilized to monitor the effect induced by the activator, e.g., protons, on the gated ion channel containing cell.

The gated ion channel containing cells can be subjected to the action of protons by adjustment of the pH to an acidic level using any convenient acid or buffer, including organic acids such as formic acid, acetic acid, citric acid, ascorbic acid, 2-morpholinoethanesulfonic acid (MES) and lactic acid, and inorganic acids such as hydrochloric acid, hydrobromic acid and nitric acid, perchloric acid and phosphoric acid.

In the methods of the invention, the current flux induced by the activator, e.g., protons, across the membrane of the gated ion channel containing cell can be monitored by electrophysiological methods, for example patch clamp or two-electrode voltage clamp techniques.

Alternatively, the change in membrane potential induced by gated ion channel activators, e.g., protons of the gated ion channel containing cells can be monitored using fluorescence methods. When using fluorescence methods, the gated ion channel containing cells are incubated with a membrane potential indicating agent that allows for a determination of changes in the membrane potential of the cells, caused by the added activators, e.g., protons. Such membrane potential indicating agents include fluorescent indicators, preferably DiBAC₄(3), DiOC5(3), DiOC2(3), DiSBAC2(3) and the FMP (FLIPR membrane potential) dyes (Molecular Devices).

In another alternative embodiment, the change in gated ion channel activity induced by activators, e.g., protons, on the gated ion channel can be measured by assessing changes in the intracellular concentration of certain ions, e.g., calcium, sodium, potassium, magnesium, protons, and chloride in cells by fluorescence. Fluorescence assays can be performed in multi-well plates using plate readers, e.g., FLIPR assay (Fluorescence Image Plate Reader; available from Molecular Devices), e.g. using fluorescent calcium indicators, e.g. as described in, for example, Sullivan E., et al. (1999) Methods Mol. Biol. 114:125-33, Jerman, J. C., et al. (2000) Br J Pharmacol 130 (4):916-22, and U.S. Pat. No. 6,608,671, the contents of each of which are incorporated herein by reference. When using such fluorescence methods, the gated ion channel containing cells are incubated with a selective ion indicating agent that allows for a determination of changes in the intracellular concentration of the ion, caused by the added activators, e.g., protons. Such ion indicating agents include fluorescent calcium indicators, preferably Fura-2, Fluo-3, Fluo-4, Fluo4FF, Fluo-5F, Fluo-5N, Calcium Green, Fura-Red, Indo-1, Indo-5F, and rhod-2, fluorescent sodium indicators, preferably SBFI, Sodium Green, CoroNa Green, fluorescent potassium indicators, preferably PBFI, CD222, fluorescent magnesium indicators, preferably Mag-Fluo-4, Mag-Fura-2, Mag-Fura-5, Mag-Fura-Red, Mag-indo-1, Mag-rho-2, Magnesium Green, fluorescent chloride indicators, preferably SPQ, Bis-DMXPQ, LZQ, MEQ, and MQAE, fluorescent pH indicators, preferably BCECF and BCPCF.

The gated ion channel antagonizing compounds of the invention show activity in concentrations below 2M, 1.5M, 1M, 500 mM, 250 mM, 100 mM, 750 μM, 500 μM, 250 μM, 100 μM, 75 μM, 50 μM, 25 μM, 10 μM, 5 μM, 2.5 μM, or below 1 μM. In its most preferred embodiment the ASIC antagonizing compounds show activity in low micromolar and the nanomolar range.

As used herein, the term “contacting” (i.e., contacting a cell e.g. a neuronal cell, with a compound) is intended to include incubating the compound and the cell together in vitro (e.g., adding the compound to cells in culture) or administering the compound to a subject such that the compound and cells of the subject are contacted in vivo. The term “contacting” is not intended to include exposure of cells to a modulator or compound that can occur naturally in a subject (i.e., exposure that can occur as a result of a natural physiological process).

A. In Vitro Assays

Gated ion channel polypeptides for use in the assays described herein can be readily produced by standard biological techniques or by chemical synthesis. For example, a host cell transfected with an expression vector containing a nucleotide sequence encoding the desired gated ion channel can be cultured under appropriate conditions to allow expression of the peptide to occur. Alternatively, the gated ion channel can be obtained by culturing a primary cell line or an established cell line that can produce the gated ion channel.

The methods of the invention can be practiced in vitro, for example, in a cell-based culture screening assay to screen compounds which potentially bind, activate or modulate gated ion channel function. In such a method, the modulating compounds can function by interacting with and eliminating any specific function of gated ion channel in the sample or culture. The modulating compounds can also be used to control gated ion channel activity in neuronal cell culture.

Cells for use in in vitro assays, in which gated ion channels are naturally present, include various cells, such as cortical neuronal cells, in particular mouse or rat cortical neuronal cells, and human embryonic kidney (HEK) cells, in particular the HEK293 cell line. For example, cells can be cultured from embryonic human cells, neonatal human cells, and adult human cells. Primary cell cultures can also be used in the methods of the invention. For example, sensory neuronal cells can also be isolated and cultured in vitro from different animal species. The most widely used protocols use sensory neurons isolated from neonatal (Eckert, et al. (1997) J Neurosci Methods 77:183-190) and embryonic (Vasko, et al. (1994) J Neurosci 14:4987-4997) rat. Trigeminal and dorsal root ganglion sensory neurons in culture exhibit certain characteristics of sensory neurons in vivo.

Alternatively, the gated ion channel, e.g., a gated channel, e.g., a proton gated ion channel, can be exogenous to the cell in question, and can in particular be introduced by recombinant DNA technology, such as transfection, microinjection or infection. Such cells include Chinese hamster ovary (CHO) cells, HEK cells, African green monkey kidney cell line (CV-1 or CV-1-derived COS cells, e.g. COS-1 and COS-7) Xenopus laevis oocytes, or any other cell lines capable of expressing gated ion channels.

The nucleotide and amino acid sequences of the gated ion channels of the invention are known in the art. For example, the sequences of the human gated channels can be found in Genbank GI Accession Nos: GI:40556387 (ENaCalpha Homo sapiens); GI:4506815 (ENaCalpha Homo sapiens); GI:4506816 (ENaCbeta Homo sapiens); GI:4506817 (ENaCbeta Homo sapiens); GI:34101281 (ENaCdelta Homo sapiens); GI:34101282 (ENaCdelta Homo sapiens); GI:42476332 (ENaCgamma Homo sapiens); GI:42476333 (ENaCgamma Homo sapiens); GI:31442760 (HINAC Homo sapiens); GI:31442761 (HINAC Homo sapiens); GI: 21536350 (ASIC1a Homo sapiens); GI:21536351 (ASIC1a Homo sapiens); GI:21536348 (ASIC1b Homo sapiens); GI:21536349 (ASIC1b Homo sapiens); GI:34452694 (ASIC2; transcript variant 1 Homo sapiens); GI:34452695 (ASIC2; isoform 1 Homo sapiens); GI:34452696 (ASIC2; transcript variant 2 Homo sapiens); GI:9998944 (ASIC2; isoform 2 Homo sapiens); GI:4757709 (ASIC3; transcript variant 1 Homo sapiens); GI:4757710 (ASIC3; isoform 1 Homo sapiens); GI:9998945 (ASIC3; transcript variant 2 Homo sapiens); GI:9998946 (ASIC3; isoform 2 Homo sapiens); GI:9998947 (ASIC3; transcript variant 3 Homo sapiens); GI: 9998948 (ASIC3; isoform 3 Homo sapiens); GI:33519441 (ASIC4; transcript variant 1 Homo sapiens); GI:33519442 (ASIC4; isoform 1 Homo sapiens); GI:33519443 (ASIC4; transcript variant 2 Homo sapiens); GI:33519444 (ASIC4; isoform 2 Homo sapiens); GI:27894283 (P2X₁ Homo sapiens); GI:4505545 (P2X₁ Homo sapiens); GI:28416917 (P2X₂; transcript variant 1 Homo sapiens); GI:25092719 (P2X₂; isoform A Homo sapiens); GI:28416922 (P2X₂; transcript variant 2 Homo sapiens); GI:28416923 (P2X₂; isoform B Homo sapiens); GI:28416916 (P2X₂; transcript variant 3 Homo sapiens); GI:7706629 (P2X₂; isoform C Homo sapiens); GI:28416918 (P2X₂; transcript variant 4 Homo sapiens); GI:25092733 (P2X₂; isoform D Homo sapiens); GI:28416920 (P2X₂; transcript variant 5 Homo sapiens); GI:28416921 (P2X₂; isoform H Homo sapiens); GI:28416919 (P2X₂; transcript variant 6 Homo sapiens); GI:27881423 (P2X₂; isoform I Homo sapiens); GI:28416924 (P2X₃ Homo sapiens); GI:28416925 (P2X₃ Homo sapiens); GI:28416926 (P2X₄; transcript variant 1 Homo sapiens); GI:28416927 (P2X₄; isoform A Homo sapiens); GI: 28416928 (P2X₄; transcript variant 2 Homo sapiens); GI:28416929 (P2X₄; isoform B Homo sapiens); GI:28416930 (P2X₄; transcript variant 3 Homo sapiens); GI:28416931 (P2X₄; isoform C Homo sapiens); GI:28416932 (P2X₅; transcript variant 1 Homo sapiens); GI:28416933 (P2X₅; isoform A Homo sapiens); GI:28416934 (P2X₅; transcript variant 2 Homo sapiens); GI:28416935 (P2X₅; isoform B Homo sapiens); GI:28416936 (P2X₅; transcript variant 3 Homo sapiens); GI:28416937 (P2X₅; isoform C Homo sapiens); GI:38327545 (P2X₆ Homo sapiens); GI:4885535 (P2X₆ Homo sapiens); GI:34335273 (P2X₇; transcript variant 1 Homo sapiens); GI:29294631 (P2X₇; isoform A Homo sapiens); GI:34335274 (P2X₇; transcript variant 2 Homo sapiens); GI:29294633 (P2X₇; isoform B Homo sapiens); GI:18375666 (TRPV1; transcript variant 1 Homo sapiens); GI:18375667 (TRPV1; vanilloid receptor subtype 1 Homo sapiens); GI:18375664 (TRPV1; transcript variant 2 Homo sapiens); GI:18375665 (TRPV1; vanilloid receptor subtype 1 Homo sapiens); GI:18375670 (TRPV1; transcript variant 3 Homo sapiens); GI:18375671 (TRPV1; vanilloid receptor subtype 1 Homo sapiens); GI:18375668 (TRPV1; transcript variant 4 Homo sapiens); GI:18375669 (TRPV1; vanilloid receptor subtype 1 Homo sapiens); GI:7706764 (VRL-1; transcript variant 1 Homo sapiens); GI:7706765 (VRL-1; vanilloid receptor-like protein 1 Homo sapiens); GI:22547178 (TRPV2; transcript variant 2 Homo sapiens); GI:20127551 (TRPV2; vanilloid receptor-like protein 1 Homo sapiens); GI:22547183 (TRPV4; transcript variant 1 Homo sapiens); GI:22547184 (TRPV4; isoform A Homo sapiens); GI:22547179 (TRPV4; transcript variant 2 Homo sapiens); GI:22547180 (TRPV4; isoform B Homo sapiens); GI:21361832 (TRPV5 Homo sapiens); GI:17505200 (TRPV5 Homo sapiens); GI:21314681 (TRPV6 Homo sapiens); GI:21314682 (TRPV6 Homo sapiens); GI: 34452696 (ACCN1; transcript variant 2; Homo sapiens). The contents of each of these records are incorporated herein by reference. Additionally, sequences for channels of other species are readily available and obtainable by those skilled in the art.

A nucleic acid molecule encoding a gated ion channel for use in the methods of the present invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

Expression vectors, containing a nucleic acid encoding a gated ion channel, e.g., a gated ion channel subunit protein, e.g., αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6 protein, TRPA1 and TRPM8 (or a portion thereof) are introduced into cells using standard techniques and operably linked to regulatory sequence. Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Baculovirus vectors available for expression of proteins in cultured insect cells (e.g., Sf 9 cells) include the pAc series (Smith et al., 1983, Mol. Cell. Biol. 3:2156-2165) and the pVL series (Lucklow and Summers, 1989, Virology 170:31-39).

Examples of mammalian expression vectors include pCDM8 (Seed, 1987, Nature 329:840), pMT2PC (Kaufman et al., 1987, EMBO J. 6:187-195), pcDNA3. When used in mammalian cells, the expression vector's control functions are often provided by viral regulatory elements. For example, commonly used promoters are derived from polyoma, Adenovirus 2, cytomegalovirus and Simian Virus 40. For other suitable expression systems for eukaryotic cells see chapters 16 and 17 of Sambrook et al.

B. In Vivo Assays

The activity of the compounds of the invention as described herein to modulate one or more gated ion channel activities (e.g., a gated ion channel modulator, e.g., a compound of the invention) can be assayed in an animal model to determine the efficacy, toxicity, or side effects of treatment with such an agent. Alternatively, an agent identified as described herein can be used in an animal model to determine the mechanism of action of such an agent.

Animal models for determining the ability of a compound of the invention to modulate a gated ion channel biological activity are well known and readily available to the skilled artisan. Examples of animal models for pain and inflammation include, but are not limited to the models listed in Table 2. Animal models for investigating neurological disorders include, but are not limited to, those described in Morris et al., (Learn. Motiv. 1981; 12: 239-60) and Abeliovitch et al., (Cell 1993; 75: 1263-71). An example of an animal model for investigating mental and behavioral disorders is the Geller-Seifter paradigm, as described in Psychopharmacology (Berl). 1979 Apr. 11; 62 (2):117-21.

Genitourinary models include methods for reducing the bladder capacity of test animals by infusing either protamine sulfate and potassium chloride (See, Chuang, Y. C. et al., Urology 61 (3): 664-670 (2003)) or dilute acetic acid (See, Sasaki, K. et al., J. Urol. 168 (3): 1259-1264 (2002)) into the bladder. For urinary tract disorders involving the bladder using intravesically administered protamine sulfate as described in Chuang et al. (2003) Urology 61: 664-70. These methods also include the use of a well accepted model of for urinary tract disorders involving the bladder using intravesically administered acetic acid as described in Sasaki et al. (2002) J. Urol. 168: 1259-64. Efficacy for treating spinal cord injured patients can be tested using methods as described in Yoshiyama et al. (1999) Urology 54: 929-33.

Animal models of neuropathic pain based on injury inflicted to a nerve (mostly the sciatic nerve) are described in Bennett et al., 1988, Pain 33:87-107; Seltzer et al., 1990, Pain 43:205-218; Kim et al., 1992, Pain 50:355-363; Decosterd et al., 2000, Pain 87:149-158 and DeLeo et al., 1994, Pain 56:9-16. There are also models of diabetic neuropathy (STZ induced diabetic neuropathy—Courteix et al., 1994, Pain 57:153-160) and drug induced neuropathies (vincristine induced neuropathy—Aley et al., 1996, Neuroscience 73: 259-265; oncology-related immunotherapy, anti-GD2 antibodies—Slart et al., 1997, Pain 60:119-125). Acute pain in humans can be reproduced using in murine animals chemical stimulation: Martinez et al., Pain 81: 179-186; 1999 (the writhing test—intraperitoneal acetic acid in mice), Dubuisson et al. Pain 1977; 4: 161-74 (intraplantar injection of formalin). Other types of acute pain models are described in Whiteside et al., 2004, Br J Pharmacol 141:85-91 (the incisional model, a post-surgery model of pain) and Johanek and Simone, 2004, Pain 109:432-442 (a heat injury model). An animal model of inflammatory pain using complete Freund's adjuvant (intraplantar injection) is described in Jasmin et al., 1998, Pain 75: 367-382. Intracapsular injection of irritant agents (complete Freund's adjuvant, iodoacetate, capsaicine, urate crystals, etc.) is used to develop arthritis models in animals (Fernihough et al., 2004, Pain 112:83-93; Coderre and Wall, 1987, Pain 28:379-393; Otsuki et al., 1986, Brain Res. 365:235-240). A stress-induced hyperalgesia model is described in Quintero et al., 2000, Pharmacology, Biochemistry and Behavior 67:449-458. Further animal models for pain are considered in an article of Walker et al. 1999 Molecular Medicine Today 5:319-321, comparing models for different types of pain, which are acute pain, chronic/inflammatory pain and chronic/neuropathic pain, on the basis of behavioral signs. Animal models for depression are described by E. Tatarczynska et al., Br. J. Pharmacol. 132 (7): 1423-1430 (2001) and P. J. M. Will et al., Trends in Pharmacological Sciences 22 (7):331-37 (2001)); models for anxiety are described by D. Treit, “Animal Models for the Study of Anti-anxiety Agents: A Review,” Neuroscience & Biobehavioral Reviews 9 (2):203-222 (1985). Additional animal models for pain are also described herein in the Exemplification section.

Gastrointestinal models can be found in: Gawad, K. A., et al., Ambulatory long-term pH monitoring in pigs, Surg Endosc, (2003); Johnson, S. E. et al., Esophageal Acid Clearance Test in Healthy Dogs, Can. J. Vet. Res. 53 (2): 244-7 (1989); and Cicente, Y. et al., Esophageal Acid Clearance: More Volume-dependent Than Motility Dependent in Healthy Piglets, J. Pediatr. Gastroenterol. Nutr. 35 (2): 173-9 (2002). Models for a variety of assays can be used to assess visceromotor and pain responses to rectal distension. See, for example, Gunter et al., Physiol. Behav., 69 (3): 379-82 (2000), Depoortere et al., J. Pharmacol. and Exp. Ther., 294 (3): 983-990 (2000), Morteau et al., Fund. Clin. Pharmacol., 8 (6): 553-62 (1994), Gibson et al., Gastroenterology (Suppl. 1), 120 (5): A19-A20 (2001) and Gschossmann et al., Eur. J. Gastro. Hepat., 14 (10): 1067-72 (2002) the entire contents of which are each incorporated herein by reference.

Gastrointestinal motility can be assessed based on either the in vivo recording of mechanical or electrical events associated intestinal muscle contractions in whole animals or the activity of isolated gastrointestinal intestinal muscle preparations recorded in vitro in organ baths (see, for example, Yaun et al., Br. J. Pharmacol., 112 (4):1095-1100 (1994), Jin et al., J. Pharm. Exp. Ther., 288 (1): 93-97 (1999) and Venkova et al., J. Pharm. Exp. Ther., 300 (3): 1046-1052 (2002)). Tatersall et al. and Bountra et al., European Journal of Pharmacology, 250: (1993) R5 and 249:(1993) R3-R4 and Milano et al., J. Pharmacol. Exp. Ther., 274 (2): 951-961 (1995)).

TABLE 2

| | | | |
|---|---|---|---|
| | Modality | | Non-limiting examples of potential clinical |
| Model Name | tested | Brief Description | indications (Reference) |

ACUTE PHASIC PAIN

| | | | |
|---|---|---|---|
| Tail-flick | Thermal | Tip of tail of rats is immersed if hot water and time to | Acute nociceptive pain |
| | | withdrawal from water is measured. Alternatively, a | (Hardy et al. Am J Physiol 1957; 189: 1-5.; |
| | | radiant heat source is applied to the tail and time to | Ben-Bassat et al. Arch Intern Pharmacodyn |
| | | withdrawal is determined. Analgesic effect is evidenced by | Ther 1959; 122: 434-47.) |
| | | a prolongation of the latency period | |
| hot-plate | Thermal | Rats walk over a heated surface with increasing | Acute nociceptive pain |
| | | temperature and observed for specific nociceptive | (Woolfe et al. J Pharmacol Exp Ther 1944; |
| | | behavior such paw licking, jumping. Time to appearance | 80: 300-7.) |
| | | of such behavior is measured. Analgesic effects are | |
| | | evidenced by a prolonged latency. | |
| Hargreaves | Thermal | A focused beam of light is projected onto a small surface | Acute nociceptive pain |
| Test | | of the hind leg of a rat with increasing temperature. Time | (Yeomans et al. Pain 1994; 59: 85-94.) |
| | | to withdrawal is measured. Analgesic effect translates into | |
| | | a prolonged latency | |
| Pin Test or | Mechanical | An increasing calibrated pressure is applied to the paw of | Acute nociceptive pain |
| Randall Selitto | | rats with a blunt pin. Pressure intensity is measured. | (Green et al. Br J Pharmacol 1951; 6: 572- |
| | | Alternatively increased pressure is applied to the paw | 85.; Randall et al. Arch Int Pharmacodyn |
| | | using a caliper until pain threshold is reached and animals | Ther 1957; 111: 409-19) |
| | | withdraw the paw. | |

HYPERALGESIA MODELS/CHRONIC INFLAMMATORY PAIN MODELS

| | | | |
|---|---|---|---|
| Hargreaves or | Thermal | A sensitizing agent (e.g, complete Freund's adjuvant | Chronic pain associated with tissue |
| Randal & Selitto | and/or | (CFA), carrageenin, turpentine etc.) is injected into the | inflammation, e.g. post-surgical pain, |
| | mechanical | paw of rats creating a local inflammation and sensitivities | (Hargreaves et al. Pain 1988; 32: 77-88.) |
| | | to mechanical (Randall & Selitto) and/or therma | Randall L O, Selitto J J. Arch Int |
| | | (Hargreaves)l stimulation are measured with comparison | Pharmacodyn1957; 3: 409-19. |
| | | to the contralateral non-sensitized paw | |
| Yeomans model | Thermal | Rat hind paw in injected with capsaicin, a sensitizing | Chronic pain associated with tissue |
| | | agent for small C-fibers or DMSO, a sensitizing agent for | inflammation, e.g. post-surgical pain |
| | | A-delta fibers. A radiant heat is applied with different | (Yeomans et al. Pain 1994; 59: 85-94.; |
| | | gradient to differentially stimulate C-fibers or A-delta fibers | Otsuki et al. Brain Res 1986; 365: 235- |
| | | and discriminate between the effects mediated by both | 240.) |
| | | pathways | |

CHRONIC MALIGNANT PAIN (CANCER PAIN)

| | | | |
|---|---|---|---|
| Bone Cancer | Thermal | In this model, osteolytic mouse sarcoma NCTC2472 cells | Bone cancer pain |
| Model | and/or | are used to induce bone cancer by injecting tumor cells | (Schwei et al., J. Neurosci. 1999; 19: |
| | mechanical | into the marrow space of the femur bone and sealing the | 10886-10897.) |
| | | injection site | |
| Cancer invasion | Thermal | Meth A sarcoma cells are implanted around the sciatic | Malignant neuropathic pain |
| pain model | and/or | nerve in BALB/c mice and these animals develop signs of | (Shimoyama et al., Pain 2002; 99: 167- |
| (CIP) | mechanical | allodynia and thermal hyperalgesia as the tumor grows, | 174.) |
| | | compressing the nerve. Spontaneous pain (paw lifting) is | |
| | | also visible. | |

CHRONIC NON-MALIGNANT PAIN

| | | | |
|---|---|---|---|
| Muscle Pain | Thermal | Repeated injections of acidic saline into one | Fibromyalgia |
| | and/or | gastrocnemius muscle produces bilateral, long-lasting | (Sluka et al. Pain 2003; 106: 229-239.) |
| | mechanical | mechanical hypersensitivity of the paw (i.e. hyperalgesia) | |
| | | without associated tissue damage | |
| UV-irradiation | Thermal | Exposure of the rat hind paw to UV irradiation produces | Inflammatory pain associated with first- and |
| | and/or | highly reliable and persistent allodynia. Various irradiation | second-degree burns. |
| | mechanical | periods with UV-B produce skin inflammation with | (Perkins et al. Pain 1993; 53: 191-197.) |
| | | different time courses | |

CHRONIC NEUROPATHIC PAIN

| | | | |
|---|---|---|---|
| Chronic | Mostly | Loose chronic ligature of the sciatic nerve. Thermal or | Clinical Neuropathic pain: nerve |
| Constriction | mechanical | mechanical sensitivities are tested using Von Frey hairs or | compression and direct mechanical |
| Injury (CCI) or | but aso | the paw withdrawal test (Hargreaves) | neuronal damage might be relevant clinical |
| Bennett and Xie | thermal | | comparisons |
| model | | | (Bennett & Xie, Neuropharmacology 1984; |
| | | | 23: 1415-1418.) |
| Chung's model | Mostly | Tight ligation of one of the two spinal nerves of the sciatic | Same as above: root compression might be |
| or Spinal Nerve | mechanical | nerve. Thermal or mechanical sensitivities are tested | a relevant clinical comparison |
| Ligation model | but aso | using Von Frey hairs or the paw withdrawal test | (Kim and Chung, Pain 1990; 41: 235-251.) |
| (SNL) | thermal | (Hargreaves) | |

Alternatively, the compounds can also be assayed in non-human transgenic animals containing exogenous sequences encoding one or more gated ion channels. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals.

A homologous recombinant animal can also be used to assay the compounds of the invention. Such animals can be generated according to well known techniques (see, e.g., Thomas and Capecchi, 1987, Cell 51:503; Li et al., 1992, Cell 69:915; Bradley, Teratocarcinomas and Embryonic Stem Cells: A Practical Approach, Robertson, Ed., IRL, Oxford, 1987, pp. 113-152; Bradley (1991) Current Opinion in Bio/Technology 2:823-829 and in PCT Publication NOS. WO 90/11354, WO 91/01140, WO 92/0968, and WO 93/04169).

Other useful transgenic non-human animals can be produced which contain selected systems which allow for regulated expression of the transgene (see, e.g., Lakso et al. (1992) Proc. Natl. Acad. Sci. USA 89:6232-6236). Another example of a recombinase system is the FLP recombinase system of Saccharomyces cerevisiae (O'Gorman et al., 1991, Science 251:1351-1355).

### Diagnostics

The AISC imaging agents of the invention, e.g., the radiolabelled compounds of Formula I, e.g., radiolabelled Compound A or radiolabelled Compound B, can be used as a targeting molecules in radiopharmaceutical formulations of the invention. These imaging agents bind to, complexes with or react with the ion channel (e.g., ASIC) sought to be bound or localized to.

The agents of Formulae I and II may be used in connection with methods for imaging an internal region of a patient and/or diagnosing the presence or absence of a disease in a patient by the application and/or detection of an energy source. Exemplary imaging agents include contrast agents for use in connection with ultrasound, magnetic resonance imaging, radionuclide imaging, or x-ray (including computed tomography) imaging of a patient, and the compositions described herein.

Magnetic resonance imaging (MRI) may be used for producing cross-sectional images of the body in a variety of scanning planes, e.g., axial, coronal, sagittal or orthogonal without exposure to radiation. MRI employs a magnetic field, radio frequency energy and magnetic field gradients to make images of the body. The contrast or signal intensity differences between tissues mainly reflect the TI (longitudinal) and T2 (transverse) relaxation values and the proton density, which generally corresponds to the free water content, of the tissues. The T1 and T2 relaxation rates may be altered by the presence of a paramagnetic ion, for example Gd, Fe, or Cu.

MRI generally requires the use of contrast agents to assist in differentiation of the tissue of interest from the surrounding tissues in the resulting image. In the past, attention has focused primarily on paramagnetic contrast agents for MRI. Paramagnetic contrast agents involve materials which contain unpaired electrons. The unpaired electrons act as small magnets within the main magnetic field to increase the rate of longitudinal (T1) and transverse (T2) relaxation. Paramagnetic contrast agents typically comprise metal ions such as transition metal ions, which provide a source of unpaired electrons. However, since these metal ions are also generally highly toxic, the ions are typically chelated.

Ultrasound is another valuable diagnostic imaging technique and provides certain advantages over other diagnostic techniques. Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one megahertz (mHz) to ten mHz. The detected waves can be integrated into an image which is quantitated and the quantitated waves converted into an image of the tissue being studied. Ultrasound also generally involves the use of contrast agents such as suspensions of solid particles, emulsified liquid droplets, and gas-filled bubbles or vesicles.

The imaging agents of the present invention may be adapted for use in the aforementioned imaging and diagnostic techniques for the imaging and/or detection of a variety of diseases and disorders in vivo. Such diseases include pain (e.g., cutaneous pain, somatic pain, visceral pain, neuropathic pain, acute pain and chronic pain); inflammatory disorders (e.g., inflammatory disorders of the musculoskeletal and connective tissue system, the respiratory system, the circulatory system, the genitourinary system, the gastrointestinal system or the nervous system); neurological disorders (e.g., schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, shingles, Huntington's chorea, Parkinson disease, anxiety disorders, panic disorders, phobias, anxiety hyteria, generalized anxiety disorder, and neurosis); diseases or disorders of the genitourinary system (e.g., cystitis, urinary tract infections, glomerulonephritis, polycystic kidney disease, kidney stones and cancers of the genitourinary system); and diseases or disorders of the gastrointestinal system (e.g., gastritis, duodenitis, irritable bowel syndrome, colitis, Crohn's disease, ulcers and diverticulitis).

In another embodiment, the imaging agents of the present invention may be adapted for use in the aforementioned imaging and diagnostic techniques for the imaging and/or detection of bone cancer. Thus, the radiolabelled compounds of Formula I, e.g., radiolabelled Compound A or radiolabelled Compound B can be used as an imaging agent for the imaging and/or detection of bone cancer.

In still other embodiments, the imaging agents of the present invention may be adapted for use in the aforementioned imaging and diagnostic techniques for the imaging and/or detection of diseases and disorders related to the skin, heart, muscles, eyes, ears, tongue, lungs, and bones of a subject.

In still other embodiments, the imaging agents of the present invention may be adapted for use in the aforementioned imaging and diagnostic techniques for the imaging and/or detection of a variety of diseases and disorders related to mechanosensation (cutaneous and visceral), cardiac pain, myocardial ischemia, angina pectoris, cardiovascular homeostasis, hypotension, hypertension, tachycardia, angina pectoris, and ischemia.

In another embodiment, the imaging agents of the present invention may be adapted for use in the aforementioned imaging and diagnostic techniques for the imaging and/or detection of several painful bone pathologies such as metastatic bone disease, Paget's disease of bones, osteoporosis, fibrous dysplasia, osteogenesis imperfecta, or bone metastases.

In still another embodiment, the imaging agents of the present invention may be adapted for use in the aforementioned imaging and diagnostic techniques for in vitro imaging. For example, the compounds of Formulae I and II can be used for in vitro imaging of tissue samples, such as human tissue samples. The tissue samples may be healthy or diseased. The tissue samples may, for example, result from a biopsy or autopsy. In one embodiment, Compound A or Compound B, wherein Compound A or Compound B may or may not have an atom that is an isotope, can be used for in vitro imaging of a tissue sample.

### Competitive Binding Assays

The compounds of the invention (i.e., compounds of the Formulae I, II and III) can be used for the purposes of screening for inhibitors of ion channel activity by using a competitive binding assay wherein more than one chemical entity competes for a binding site. For example, in a competitive binding assay of the invention, competition occurs between potential ion channel (e.g., ASIC) inhibitors (i.e., compounds being investigated for inhibitory activity) and a standard known for ion channel (e.g., ASIC) inhibitory activity, in which the standard has been tagged by a radiolabel. In a particular embodiment, Compound A or Compound B, wherein one or more of the atoms of Compound A or Compound B may or may not be an isotope, can be used in a competitive binding assay to identify modulators of ASIC activity. It is understood that the theory of competitive binding is well known to one skilled in the art of pharmacology. The compounds identified by this assay may have utility for the prevention and treatment of neurological disorders relating to pain, inflammation, cardiovascular disorders, respiratory conditions, genitourinary disorders, gastrointestinal disorders, cancers and neurological disorders.

### Pharmaceutical Compositions

The present invention also provides pharmaceutical compositions. Such compositions comprise a therapeutically (or prophylactically) effective amount of a gated ion channel modulator, and preferably one or more compounds of the invention described above, and a pharmaceutically acceptable carrier or excipient. Suitable pharmaceutically acceptable carriers include, but are not limited to, saline, buffered saline, dextrose, water, glycerol, ethanol, and combinations thereof. The carrier and composition can be sterile. The formulation should suit the mode of administration.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: sugars, such as lactose, glucose, dextrose and sucrose; starches, such as corn starch and potato starch; cellulose, and its derivatives, such as sodium carboxymethyl cellulose, ethyl cellulose, methylcellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, castor oil, tetraglycol, and soybean oil; glycols, such as propylene glycol; polyols, such as glycerin, sorbitol, mannitol and polyethylene glycol; esters, such as ethyl oleate, esters of polyethylene glycol and ethyl laurate; agar; buffering agents, such as magnesium hydroxide, sodium hydroxide, potassium hydroxide, carbonates, triethylanolamine, acetates, lactates, potassium citrate and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, suitable pharmaceutically acceptable carriers for the compounds of the invention include water, saline, buffered saline, and HPβCD (hydroxypropyl β-cyclodextrin).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol and derivatives such as vitamin E tocopherol, and the like; and metal chelating agents, such as citric acid, ethylenediamine tetraacetic acid (EDTA), sorbitol, tartaric acid, phosphoric acid, sodium citrate and the like.

Suitable pharmaceutically acceptable carriers include but are not limited to water, salt solutions (e.g., NaCl), alcohols, gum arabic, vegetable oils, benzyl alcohols, polyethylene glycols, gelatin, carbohydrates such as lactose, amylose or starch, cyclodextrin, magnesium stearate, talc, silicic acid, viscous paraffin, perfume oil, fatty acid esters, hydroxymethylcellulose, polyvinyl pyrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. The pharmaceutically acceptable carriers can also include a tonicity-adjusting agent such as dextrose, glycerine, mannitol and sodium chloride.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition can be formulated in accordance with the routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients can be mixed prior to administration.

The pharmaceutical compositions of the invention can also include an agent which controls release of the gated ion channel modulator compound, thereby providing a timed or sustained release composition.

The present invention also relates to prodrugs of the gated ion channel modulators disclosed herein, as well as pharmaceutical compositions comprising such prodrugs. For example, compounds of the invention which include acid functional groups or hydroxyl groups can also be prepared and administered as a corresponding ester with a suitable alcohol or acid. The ester can then be cleaved by endogenous enzymes within the subject to produce the active agent.

Formulations of the present invention include those suitable for oral, nasal, topical, mucous membrane, transdermal, buccal, sublingual, rectal, vaginal and/or parenteral administration. The formulations can conveniently be presented in unit dosage form and can be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention can also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions of the present invention, such as dragees, capsules, pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions that can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms can contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration can be presented as a suppository, which can be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate. Dosage forms for the topical or transdermal administration of a compound of this invention include powders, sprays, ointments, pastes, creams, lotions, gels, solutions, patches and inhalants. The active compound can be mixed under sterile conditions with a pharmaceutically acceptable carrier, and with any preservatives, buffers, or propellants that can be required.

The ointments, pastes, creams and gels can contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers that can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

### Methods of Administration

The invention provides a method of treating a condition mediated by gated ion channel activity in a subject, including, but not limited to, pain, inflammatory disorders, neurological disorders, gastrointestinal disorders and genitourinary disorders. The method comprises the step of administering to the subject a therapeutically effective amount of a gated ion channel modulator (e.g., a metabolite having the Formula III or IV, e.g., Compound L). The condition to be treated can be any condition which is mediated, at least in part, by the activity of a gated ion channel (e.g., ASIC1a and/or ASIC3).

The quantity of a given compound to be administered will be determined on an individual basis and will be determined, at least in part, by consideration of the individual's size, the severity of symptoms to be treated and the result sought. The gated ion channel activity modulators described herein can be administered alone or in a pharmaceutical composition comprising the modulator, an acceptable carrier or diluent and, optionally, one or more additional drugs.

These compounds can be administered to humans and other animals for therapy by any suitable route of administration. The gated ion channel modulator can be administered subcutaneously, intravenously, parenterally, intraperitoneally, intradermally, intramuscularly, topically, enterally (e.g., orally), rectally, nasally, buccally, sublingually, systemically, vaginally, by inhalation spray, by drug pump or via an implanted reservoir in dosage formulations containing conventional non-toxic, physiologically acceptable carriers or vehicles. The preferred method of administration is by oral delivery. The form in which it is administered (e.g., syrup, elixir, capsule, tablet, solution, foams, emulsion, gel, sol) will depend in part on the route by which it is administered. For example, for mucosal (e.g., oral mucosa, rectal mucosa, intestinal mucosa, bronchial mucosa) administration, nose drops, aerosols, inhalants, nebulizers, eye drops or suppositories can be used. The compounds and agents of this invention can be administered together with other biologically active agents, such as analgesics, e.g., opiates, anti-inflammatory agents, e.g., NSAIDs, anesthetics and other agents which can control one or more symptoms or causes of a gated ion channel mediated condition.

In a specific embodiment, it can be desirable to administer the agents of the invention locally to a localized area in need of treatment; this can be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers. For example, the agent can be injected into the joints or the urinary bladder.

The compounds of the invention can, optionally, be administered in combination with one or more additional drugs which, for example, are known for treating and/or alleviating symptoms of the condition mediated by a gated ion channel (e.g., ASIC1a and/or ASIC3). The additional drug can be administered simultaneously with the compound of the invention, or sequentially. For example, the compounds of the invention can be administered in combination with at least one of an analgesic, an anti-inflammatory agent, an anesthetic, a corticosteroid (e.g., dexamethasone, beclomethasone diproprionate (BDP) treatment), an anti-convulsant, an antidepressant, an anti-nausea agent, an anti-psychotic agent, a cardiovascular agent (e.g., a beta-blocker) or a cancer therapeutic. In certain embodiments, the compounds of the invention are administered in combination with a pain drug. As used herein the phrase, “pain drugs” is intended to refer to analgesics, anti-inflammatory agents, anesthetics, corticosteroids, antiepileptics, barbiturates, antidepressants, and marijuana.

The combination treatments mentioned above can be started prior to, concurrent with, or after the administration of the compositions of the present invention. Accordingly, the methods of the invention can further include the step of administering a second treatment, such as a second treatment for the disease or disorder or to ameliorate side effects of other treatments. Such second treatment can include, e.g., anti-inflammatory medication and any treatment directed toward treating pain. Additionally or alternatively, further treatment can include administration of drugs to further treat the disease or to treat a side effect of the disease or other treatments (e.g., anti-nausea drugs, anti-inflammatory drugs, anti-depressants, anti-psychiatric drugs, anti-convulsants, steroids, cardiovascular drugs, and cancer chemotherapeutics).

As used herein, an “analgesic” is an agent that relieves or reduces pain or any signs or symptoms thereof (e.g., hyperalgesia, allodynia, dysesthesia, hyperesthesia, hyperpathia, paresthesia) and can also result in the reduction of inflammation, e.g., an anti-inflammatory agent. Analgesics can be subdivided into NSAIDs (non-steroidal-anti-inflammatory drugs), narcotic analgesics, including opioid analgesics, and non-narcotic analgesics. NSAIDs can be further subdivided into non-selective COX (cyclooxygenase) inhibitors, and selective COX2 inhibitors. Opioid analgesics can be natural, synthetic or semi-synthetic opioid analgesics, and include for example, morphine, codeine, meperidine, propxyphen, oxycodone, hydromorphone, heroine, tramadol, and fentanyl. Non-narcotic analgesics (also called non-opioid) analgesics include, for example, acetaminophen, clonidine, NMDA antagonists, vanilloid receptor antagonists (e.g., TRPV1 antagonists), pregabalin, endocannabinoids and cannabinoids. Non-selective COX inhibitors include, but are not limited to acetylsalicylic acid (ASA), ibuprofen, naproxen, ketoprofen, piroxicam, etodolac, and bromfenac. Selective COX2 inhibitors include, but are not limited to celecoxib, valdecoxib, parecoxib, and etoricoxib.

As used herein an “anesthetic” is an agent that interferes with sense perception near the site of administration, a local anesthetic, or result in alteration or loss of consciousness, e.g., systemic anesthetic agents. Local anesthetics include but are not limited to lidocaine and buvicaine.

Non-limiting examples of antiepileptic agents are carbamazepine, phenyloin and gabapentin. Non-limiting examples of antidepressants are amitriptyline and desmethylimiprimine.

Non-limiting examples of anti-inflammatory drugs include corticosteroids (e.g., hydrocortisone, cortisone, prednisone, prednisolone, methyl prednisone, triamcinolone, fluprednisolone, betamethasone and dexamethasone), salicylates, NSAIDs, antihistamines and H₂ receptor antagonists.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Regardless of the route of administration selected, the compounds of the present invention, which can be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention can be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the subject being treated, and like factors well known in the medical arts.

A physician or veterinarian having ordinary skill in the art can readily determine and prescribe the effective amount of the pharmaceutical composition required. For example, dosages of a compound of the invention can be determined by deriving dose-response curves using an animal model for the condition to be treated. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a subject, when used for the indicated analgesic effects, will range from about 0.0001 to about 100 mg per kilogram of body weight per day, more preferably from about 0.01 to about 100 mg per kg per day, and still more preferably from about 1.0 to about 50 mg per kg per day. An effective amount is that amount that treats a gated ion channel-associated state or gated ion channel disorder.

If desired, the effective daily dose of the active compound can be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Kits

The present invention also provides kits for use by a consumer for treating disease. The kits comprise a) a pharmaceutical composition comprising a compound of the invention and a pharmaceutically acceptable carrier, vehicle or diluent; and, optionally, b) instructions describing a method of using the pharmaceutical composition for treating the specific disease. The instructions may also indicate that the kit is for treating disease while substantially reducing the concomitant liability of adverse effects associated with estrogen administration.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Methods of Treatment

The above compounds (e.g., a metabolite having the Formula III or IV, e.g., Compound L) can be used for administration to a subject for the modulation of a gated ion channel-mediated activity, involved in, but not limited to, pain, inflammatory disorders, neurological disorders, and any abnormal function of cells, organs, or physiological systems that are modulated, at least in part, by a gated ion channel-mediated activity. Additionally, it is understood that the compounds can also alleviate or treat one or more additional symptoms of a disease or disorder discussed herein.

Accordingly, in one aspect, the compounds of the invention can be used to treat pain, including acute, chronic, malignant and non-malignant somatic pain (including cutaneous pain and deep somatic pain), visceral pain, and neuropathic pain. It is further understood that the compounds can also alleviate or treat one or more additional signs or symptoms of pain and sensory deficits (e.g., hyperalgesia, allodynia, dysesthesia, hyperesthesia, hyperpathia, paresthesia).

In some embodiments of this aspect of the invention, the compounds of the invention can be used to treat somatic or cutaneous pain associated with injuries, inflammation, diseases and disorders of the skin and related organs including, but not limited to, cuts, burns, lacerations, punctures, incisions, surgical pain, post-operative pain, orodental surgery, psoriasis, eczema, dermatitis, and allergies. The compounds of the invention can also be used to treat somatic pain associated with malignant and non-malignant neoplasm of the skin and related organs (e.g., melanoma, basal cell carcinoma).

In other embodiments of this aspect of the invention, the compounds of the invention can be used to treat deep somatic pain associated with injuries, inflammation, diseases and disorders of the musculoskeletal and connective tissues including, but not limited to, arthralgias, myalgias, fibromyalgias, myofascial pain syndrome, dental pain, lower back pain, pain during labor and delivery, surgical pain, post-operative pain, headaches, migraines, idiopathic pain disorder, sprains, bone fractures, bone injury, osteoporosis, severe burns, gout, arthritis, osteoarthithis, myositis, and dorsopathies (e.g., spondylolysis, subluxation, sciatica, and torticollis). The compounds of the invention can also be used to treat deep somatic pain associated with malignant and non-malignant neoplasm of the musculoskeletal and connective tissues (e.g., sarcomas, rhabdomyosarcomas, and bone cancer).

In other embodiments of this aspect of the invention, compounds of the invention can be used to treat visceral pain associated with injuries, inflammation, diseases or disorders of the circulatory system, the respiratory system, the genitourinary system, the gastrointestinal system and the eye, ear, nose and throat.

For example, the compounds of the invention can be used to treat visceral pain associated with injuries, inflammation and disorders of the circulatory system associated including, but are not limited to, ischaemic diseases, ischaemic heart diseases (e.g., angina pectoris, acute myocardial infarction, coronary thrombosis, coronary insufficiency), diseases of the blood and lymphatic vessels (e.g., peripheral vascular disease, intermittent claudication, varicose veins, haemorrhoids, embolism or thrombosis of the veins, phlebitis, thrombophlebitis lymphadenitis, lymphangitis), and visceral pain associated with malignant and non-malignant neoplasm of the circulatory system (e.g., lymphomas, myelomas, Hodgkin's disease).

In another example, the compounds of the invention can be used to treat visceral pain associated with injuries, inflammation, diseases and disorders of the respiratory system including, but are not limited to, upper respiratory infections (e.g., nasopharyngitis, sinusitis, and rhinitis), influenza, pneumoniae (e.g., bacterial, viral, parasitic and fungal), lower respiratory infections (e.g., bronchitis, bronchiolitis, tracheobronchitis), interstitial lung disease, emphysema, bronchiectasis, status asthmaticus, asthma, pulmonary fibrosis, chronic obstructive pulmonary diseases (COPD), diseases of the pleura, and visceral pain associated with malignant and non-malignant neoplasm of the respiratory system (e.g., small cell carcinoma, lung cancer, neoplasm of the trachea, of the larynx).

In another example, the compounds of the invention can be used to treat visceral pain associated with injuries, inflammation and disorders of the gastrointestinal system including, but are not limited to, injuries, inflammation and disorders of the tooth and oral mucosa (e.g., impacted teeth, dental caries, periodontal disease, oral aphthae, pulpitis, gingivitis, periodontitis, and stomatitis), of the oesophagus, stomach and duodenum (e.g., ulcers, dyspepsia, oesophagitis, gastritis, duodenitis, diverticulitis and appendicitis), of the intestines (e.g., Crohn's disease, paralytic ileus, intestinal obstruction, irritable bowel syndrome, neurogenic bowel, megacolon, inflammatory bowel disease, ulcerative colitis, and gastroenteritis), of the peritoneum (e.g. peritonitis), of the liver (e.g., hepatitis, liver necrosis, infarction of liver, hepatic veno-occlusive diseases), of the gallbladder, biliary tract and pancreas (e.g., cholelithiasis, cholecystolithiasis, choledocholithiasis, cholecystitis, and pancreatitis), functional abdominal pain syndrome (FAPS), gastrointestinal motility disorders, as well as visceral pain associated with malignant and non-malignant neoplasm of the gastrointestinal system (e.g., neoplasm of the oesophagus, stomach, small intestine, colon, liver and pancreas).

In another example, the compounds of the invention can be used to treat visceral pain associated with injuries, inflammation, diseases, and disorders of the genitourinary system including, but are not limited to, injuries, inflammation and disorders of the kidneys (e.g., nephrolithiasis, glomerulonephritis, nephritis, interstitial nephritis, pyelitis, pyelonephritis), of the urinary tract (e.g. include urolithiasis, urethritis, urinary tract infections), of the bladder (e.g. cystitis, neuropathic bladder, neurogenic bladder dysfunction, overactive bladder, bladder-neck obstruction), of the male genital organs (e.g., prostatitis, orchitis and epididymitis), of the female genital organs (e.g., inflammatory pelvic disease, endometriosis, dysmenorrhea, ovarian cysts), as well as pain associated with malignant and non-malignant neoplasm of the genitourinary system (e.g., neoplasm of the bladder, the prostate, the breast, the ovaries).

In further embodiments of this aspect of the invention, compounds of the invention can be used to treat neuropathic pain associated with injuries, inflammation, diseases and disorders of the nervous system, including the central nervous system and the peripheral nervous systems. Examples of such injuries, inflammation, diseases or disorders associated with neuropathic pain include, but are not limited to, neuropathy (e.g., diabetic neuropathy, drug-induced neuropathy, radiotherapy-induced neuropathy), neuritis, radiculopathy, radiculitis, neurodegenerative diseases (e.g., muscular dystrophy), spinal cord injury, peripheral nerve injury, nerve injury associated with cancer, Morton's neuroma, headache (e.g., nonorganic chronic headache, tension-type headache, cluster headache and migraine), migraine, multiple somatization syndrome, postherpetic neuralgia (shingles), trigeminal neuralgia complex regional pain syndrome (also known as causalgia or Reflex Sympathetic Dystrophy), radiculalgia, phantom limb pain, chronic cephalic pain, nerve trunk pain, somatoform pain disorder, central pain, non-cardiac chest pain, central post-stroke pain.

In another aspect, the compounds of the invention can be used to treat inflammation associated with injuries, diseases or disorders of the skin and related organs, the musculoskeletal and connective tissue system, the respiratory system, the circulatory system, the genitourinary system and the gastrointestinal system.

In some embodiments of this aspect of the invention, examples of inflammatory conditions, diseases or disorders of the skin and related organs that can be treated with the compounds of the invention include, but are not limited to allergies, atopic dermatitis, psoriasis and dermatitis.

In other embodiments of this aspect of the invention, inflammatory conditions, diseases or disorders of the musculoskeletal and connective tissue system that can be treated with the compounds of the invention include, but are not limited to arthritis, osteoarthritis, and myositis.

In other embodiments of this aspect of the invention, inflammatory conditions, diseases or disorders of the respiratory system that can be treated with the compounds of the invention include, but are not limited to allergies, asthma, rhinitis, neurogenic inflammation, pulmonary fibrosis, chronic obstructive pulmonary disease (COPD), adult respiratory distress syndrome, nasopharyngitis, sinusitis, and bronchitis.

In still other embodiments of this aspect of the invention, inflammatory conditions, disease or disorders of the circulatory system that can be treated with the compounds of the invention include, but are not limited to, endocarditis, pericarditis, myocarditis, phlebitis, lymphadenitis and artherosclerosis.

In further embodiments of this aspect of the invention, inflammatory conditions, diseases or disorders of the genitourinary system that can be treated with the compounds of the invention include, but are not limited to, inflammation of the kidney (e.g., nephritis, interstitial nephritis), of the bladder (e.g., cystitis), of the urethra (e.g., urethritis), of the male genital organs (e.g., prostatitis), and of the female genital organs (e.g., inflammatory pelvic disease).

In further embodiments of this aspect of the invention, inflammatory conditions, diseases or disorders of the gastrointestinal system that can be treated with the compounds of the invention include, but are not limited to, gastritis, gastroenteritis, colitis (e.g., ulcerative colitis), inflammatory bowel syndrome, Crohn's disease, cholecystitis, pancreatitis and appendicitis.

In still further embodiments of this aspect of the invention, inflammatory conditions, diseases or disorders that can be treated with the compounds of the invention, but are not limited to inflammation associated with microbial infections (e.g., bacterial, viral and fungal infections), physical agents (e.g., burns, radiation, and trauma), chemical agents (e.g., toxins and caustic substances), tissue necrosis and various types of immunologic reactions and autoimmune diseases (e.g., lupus erythematosus).

In another aspect, the compounds of the invention can be used to treat injuries, diseases or disorders of the nervous system including, but not limited to neurodegenerative diseases (e.g., Alzheimer's disease, Duchenne's disease), epilepsy, multiple sclerosis, amyotrophic lateral sclerosis, stroke, cerebral ischemia, neuropathies (e.g., chemotherapy-induced neuropathy, diabetic neuropathy), retinal pigment degeneration, trauma of the central nervous system (e.g., spinal cord injury), and cancer of the nervous system (e.g., neuroblastoma, retinoblastoma, brain cancer, and glioma), and other certain cancers (e.g., melanoma, pancreatic cancer).

In further aspects of the invention, the compounds of the invention can also be used to treat other disorders of the skin and related organs (e.g., hair loss), of the circulatory system, (e.g., cardiac arrhythmias and fibrillation and sympathetic hyper-innervation), and of the genitourinary system (e.g., neurogenic bladder dysfunction and overactive bladder).

The present invention provides a method for treating a subject that would benefit from administration of a composition of the present invention. Any therapeutic indication that would benefit from a gated ion channel modulator can be treated by the methods of the invention. The method includes the step of administering to the subject a composition of the invention, such that the disease or disorder is treated.

The invention further provides a method for preventing in a subject, a disease or disorder which can be treated with administration of the compositions of the invention. Subjects “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of a disease or disorder that can be treated according the methods of the invention. For example, risk factors include family history, medication history, and history of exposure to an environmental substance which is known or suspected to increase the risk of disease. Subjects at risk for a disease or condition which can be treated with the agents mentioned herein can also be identified by, for example, any or a combination of diagnostic or prognostic assays known to those skilled in the art. Administration of a prophylactic agent can occur prior to the manifestation of symptoms characteristic of the disease or disorder, such that the disease or disorder is prevented or, alternatively, delayed in its progression.

### EXEMPLIFICATION OF THE INVENTION

The invention is further illustrated by the following examples, which could be used to examine the gated ion channel modulating activity of the compounds of the invention. The example should not be construed as further limiting. The animal models used throughout the Examples are accepted animal models and the demonstration of efficacy in these animal models is predictive of efficacy in humans.

It is noted that the compounds discussed this section are not radiolabelled.

### Example 1

### Identification of ASIC Antagonists Using Calcium-Imaging

### Cell Culture

ASIC1a expressing HEK293 cells are grown in culture medium (DMEM with 10% FBS), in polystyrene culture flasks (175 mm²) at 37° C. in a humidified atmosphere of 5% CO₂. Confluency of cells should be 80-90% on day of plating. Cells are rinsed with 10 ml of PBS and re-suspended by addition of culture medium and trituration with a 25 ml pipette.

The cells are seeded at a density of approximately 1×10⁶ cells/ml (100 μl/well) in black-walled, clear bottom, 96-well plates pre-treated with 10 mg/l poly-D-lysin (75 μl/well for ≧30 min). Plated cells were allowed to proliferate for 24 h before loading with dye.

Loading with Fluorescent Calcium Dye Fluo-4/AM

Fluo-4/AM (1 mg, Molecular Probes) is dissolved in 912 μl DMSO. The Fluo-4/AM stock solution (1 mM) is diluted with culture medium to a final concentration of 2 μM (loading solution).

The culture medium is aspirated from the wells, and 50 μl of the Fluo-4/AM loading solution is added to each well. The cells are incubated at 37° C. for 30 min.

### Calcium Measurements

After the loading period, the loading solution is aspirated and the cells are washed twice with 100 μl modified Assay Buffer (145 mM NaCl, 5 mM KCl, 5 mM CaCl₂, 1 mM MgCl₂, 10 mM HEPES, pH 7.4) to remove extracellular dye. Following the second wash, 100 μl modified Assay Buffer is added to each well and the fluorescence is measured in FLIPR™ or FlexStation™ (Molecular Devices, USA), or any other suitable equipment known to the skilled in the art.

### FLIPR Settings (ASIC1a)

Temperature: Room temperature (20-22° C.)

First addition: 50 μl test solution at a rate of 30 μl/sec and a starting height of 100 μl

Second addition: 50 μl MES solution (20 mM, 5 mM final concentration) at a rate of 35 μl/sec and a starting height of 150 μl.

Reading intervals: pre-incubation—10 sec×7 and 3 sec×3 antagonist phase—3 sec×17 and 10 sec×12

Addition plates (compound test plate and MES plate) are placed on the right and left positions in the FLIPR tray, respectively. Cell plates are placed in the middle position and the ASIC1a program is effectuated. FLIPR will then take the appropriate measurements in accordance with the interval settings above. Fluorescence obtained after stimulation is corrected for the mean basal fluorescence (in modified Assay Buffer).

### Hit Confirmation and Characterization of Active Substances

The MES-induced peak calcium response, in the presence of test substance, is expressed relatively to the MES response alone. Test substances that block the MES-induced calcium response are re-tested in triplicates. Confirmed hits are picked for further characterization by performing full dose-response curves to determine potency of each hit compound as represented by the IC₅₀ values (i.e., the concentration of the test substance which inhibits 50% of the MES-induced calcium response).

### Example 2

### Screening and Bioanalysis of ASIC Antagonists in Heterologous Expression Systems

This example describes another in vitro assessment of the activity of the compounds of the present invention.

Another example of an in vitro assessment method consists of using mammalian heterologous expression systems, which are known to the skilled in the art, and include a variety of mammalian cell lines such as COS, HEK, e.g., HEK293 and/or CHO, cells. Cell lines are transfected with gated ion channel(s) and used to perform electrophysiology as follows:

All experiments are performed at room temperature (20-25° C.) in voltage clamp using conventional whole cell patch clamp methods (Neher, E., et al. (1978) Pfluegers Arch 375:219-228).

The amplifier used is the EPC-9 (HEKA-electronics, Lambrect, Germany) run by a Macintosh G3 computer via an ITC-16 interface. Experimental conditions are set with the Pulse-software accompanying the amplifier. Data is low pass filtered and sampled directly to hard-disk at a rate of 3 times the cut-off frequency.

Pipettes are pulled from borosilicate glass using a horizontal electrode puller (Zeitz-lnstrumente, Augsburg, Germany). The pipette resistances are 2-3 MOhms in the salt solutions used in these experiments. The pipette electrode is a chloridized silver wire, and the reference is a silver chloride pellet electrode (In Vivo Metric, Healdsburg, USA) fixed to the experimental chamber. The electrodes are zeroed with the open pipette in the bath just prior to sealing.

Coverslips with the cells are transferred to a 15 μl experimental chamber mounted on the stage of an inverted microscope (IMT-2, Olympus) supplied with Nomarski optics. Cells are continuously superfused with extracellular saline at a rate of 2.5 ml/min. After giga-seal formation, the whole cell configuration is attained by suction. The cells are held at a holding voltage of −60 mV and at the start of each experiment the current is continuously measured for 45 s to ensure a stable baseline. Solutions of low pH (<7) are delivered to the chamber through a custom-made gravity-driven flowpipe, the tip of which is placed approximately 50 μm from the cell. Application is triggered when the tubing connected to the flowpipe is compressed by a valve controlled by the Pulse-software. Initially, low pH (in general, pH 6.5) is applied for 5 s every 60 s. The sample interval during application is 550 μs. After stable responses are obtained, the extracellular saline as well as the low-pH solution are switched to solutions containing the compound to be tested. The compound is present until responses of a repeatable amplitude are achieved. Current amplitudes are measured at the peak of the responses, and effect of the compounds is calculated as the amplitude at compound equilibrium divided by the amplitude of the current evoked by the pulse just before the compound was included.

The following salt solutions are used: extracellular solution (mM): NaCl (140), KCl (4), CaCl₂ (2), MgCl₂ (4), HEPES (10, pH 7.4); intracellular solution (mM): KCl (120), KOH (31), MgCl₂ (1.785), EGTA (10), HEPES (10, pH 7.2). In general, compounds for testing are dissolved in 50% DMSO at 500 fold the highest concentration used.

Furthermore, Compound A and amiloride were able to reduce the human ASIC1a pH-evoked response in CHO cells in a dose-dependent manner. However, Compound A was about 100 fold more potent.

### Example 3

### Screening and Bioanalysis of ASIC Antagonists in Xenopus laevis Oocytes

This example describes the in vitro assessment of the activity of the compounds of the present invention.

Two-electrode voltage clamp electrophysiological assays in Xenopus laevis oocytes expressing gated ion channels are performed as follows:

Oocytes are surgically removed from adult Xenopus laevis and treated for 2 h at room temperature with 1 mg/ml type I collagenase (Sigma) in Barth's solution under mild agitation. Selected oocytes at stage IV-V are defolliculated manually before nuclear microinjection of 2.5-5 ng of a suitable expression vector, such as pcDNA3, comprising the nucleotide sequence encoding a gated ion channel subunit protein. In such an experiment, the oocytes express homomultimeric proton-gated ion channels on their surface. In an alternate experiment, one, two, three or more vectors comprising the coding sequences for distinct gated ion channel subunits are co-injected in the oocyte nuclei. In the latter case, oocytes express heteromultimeric proton-gated ion channels. For example, ASIC2a and/or ASIC3 subunits in pcDNA3 vector are co-injected at a 1:1 cDNA ratio. After 2-4 days of expression at 19° C. in Barth's solution containing 50 mg/ml gentamicin and 1.8 mM CaCl₂, gated ion channels are activated by applying an acidic solution (pH<7) and currents are recorded in a two electrode voltage-clamp configuration, using an OC-725B amplifier (Warner Instruments). Currents are acquired and digitized at 500 Hz on an Apple Imac G3 computer with an A/D NB-MIO-16XL interface (National Instruments) and recorded traces are post-filtered at 100 Hz in Axograph (Axon Instruments) (Neher, E. and Sakmann, B. (1976) Nature 260:799-802). Once impaled with the microelectrodes, oocytes are continuously superfused at 10-12 ml/min with a modified Ringer's solution containing 97 mM NaCl, 2 mM KCl, 1.8 mM CaCl₂, and 10 mM HEPES brought to pH 7.4 with NaOH (Control Ringer). Test Ringer solution is prepared by replacing HEPES with MES and adjusting the pH to the desired acidic value. Compounds of the present invention are prepared in both the Control and Test Ringer solutions and applied to oocytes at room temperature through a computer-controlled switching valve system. Osmolarity of all solutions is adjusted to 235 mOsm with choline chloride. Similarly, recordings can also be acquired in an automated multichannel oocytes system as the OpusExpress™ (Molecular Devices, Sunnyvale, USA).

A summary of IC₅₀ values of compounds of the invention as acquired in the calcium mobilization assay are shown below. n=3-7. It is noted that the compounds below are not radiolabelled.

| Compound | IC₅₀ (μM) |
|---|---|
| Compound A (isomeric mixture - predominantly E isomer) | 0.7 |
| Compound A (minor Z isomer isolated) | 1.0 |
| Compound L (metabolite of Compound A) | 0.8 |
| Compound G | 0.3 |
| Compound J | 0.7 |

### Example 4

### Screening and Bioanalysis of ASIC Antagonists in Primary Cell Systems

This example describes another in vitro assessment of the inhibitory activity of the compounds of the present invention utilizing patch-clamp electrophysiology of sensory neurons in primary culture.

Sensory neurons can be isolated and cultured in vitro from different animal species. The most widely used protocols use sensory neurons isolated from neonatal (Eckert, et al. (1997) J Neurosci Methods 77:183-190) and embryonic (Vasko, et al. (1994) J Neurosci 14:4987-4997) rat. Trigeminal and dorsal root ganglion sensory neurons in culture exhibit certain characteristics of sensory neurons in vivo. Electrophysiology is performed similarly as described above in Example 2. In the voltage-clamp mode, trans-membrane currents are recorded. Compounds A and H at 1 μM inhibit the pH 6.5-induced inward current. In the current-clamp mode, change in the trans-membrane potential are recorded. Under acidic conditions (e.g., pH 6.5) the membrane depolarizes, leading to the firing of action potentials. Compounds A and H at 1 μM inhibit the acid-induced membrane depolarization and reduce the ensuing rate of action potential firing. Data collected using these procedures demonstrate that Compounds A and H effectively modulate the activity of these native sensory-neuron gated ion channels (n=3).

### Example 5

### Formalin Model—Model of Acute Tonic Pain

This example describes the in vivo assessment of the inhibitory activity of the compounds of the present invention.

A number of well-established models of pain are described in the literature and are known to the skilled in the art (see, for example, Table 2). This example describes the use of the Formalin test.

Male Sprague-Dawley rats are housed together in groups of three animals under standard conditions with unrestricted access to food and water. All experiments are conducted according to the ethical guidelines for investigations of experimental pain in conscious animals (Zimmerman, 1983)

Assessment of formalin-induced flinching behavior in normal, uninjured rats (body weight 150-180 g) was made with the use of an Automated Nociception Analyser (University of California, San Diego, USA). Briefly, this involved placing a small C-shaped metal band (10 mm wide×27 mm long) on the hindpaw of the rat to be tested. The rats (four rats were included in each testing session) were then placed in a cylindrical plexiglass observation chamber (diameter 30.5 cm and height 15 cm) for 20 min for adaptation purposes prior to being administered drug or vehicle according to the experimental paradigm being followed. After adaptation, individual rats were then gently restrained and formalin (5% in saline, 50 μl, s.c.) was injected into the plantar surface of the hindpaw using a 27 G needle. Rats were then returned to their separate observation chambers, each of which were in turn situated upon an enclosed detection device consisting of two electromagnetic coils designed to produce an electromagnetic field in which movement of the metal band could be detected. The analogue signal was then digitised and a software algorithm (LabView) applied to enable discrimination of flinching behaviour from other paw movements. A sampling interval of 1 min was used and on the basis of the resulting response patterns 5 phases of nociceptive behaviour were identified and scored: first phase (P1; 0-5 min), interphase (Int; 6-15 min), second phase (P2; 60 min), phase 2A (P2A; 16-40 min) and phase 2B (P2B; 41-60 min).

Nociceptive behavior was also determined manually every 5 min by measuring the amount of time spent in each of four behavioral categories: 0, treatment of the injected hindpaw is indistinguishable from that of the contralateral paw; 1, the injected paw has little or no weight placed on it; 2, the injected paw is elevated and is not in contact with any surface; 3, the injected paw is licked, bitten, or shaken. A weighted nociceptive score, ranging from 0 to 3 was calculated by multiplying the time spent in each category by the category weight, summing these products, and dividing by the total time for each 5 min block of time. (Coderre et al., Pain 1993; 54: 43). On the basis of the resulting response patterns, 2 phases of nociceptive behavior were identified and scored: first phase (P1; 0-5 min), interphase (Int; 6-15 min), second phase (P2; 60 min), phase 2A (P2A; 16-40 min) and phase 2B (P2B; 41-60 min).

Statistical analysis was performed using Prism™ 4.01 software package (GraphPad, San Diego, Calif., USA). The difference in response levels between treatment groups and control vehicle group was analyzed using an ANOVA followed by Bonferroni's method for post-hoc pair-wise comparisons. A p value<0.05 was considered to be significant

The effect of Compound A on pain induced by intraplantar formalin injection was investigated. Compound A was administered i.p. 30 min. before the formalin. Compound A was able to reduce the total pain score behavior (flinching, licking, biting) in phase 1 and 2 of the formalin test. These effects on both phases 1 and 2 were quite pronounced when only specific pain behaviors such as liking and biting were observed (n=6-8). A clear dose-response relationship for the Phase 2 of the total pain score can be seen with an ED₅₀ of about 12 mg/kg. In these experiments, a linear relationship between dose and plasma exposure was observed. Similar results were found for compounds B and H using the Automate Nociceptive Analyzer described above (n=6-8). These results indicate that Compounds A, B and H can block acute tonic pain induced by formalin injection in the paw.

### Example 6

### CFA Model—Model of Chronic Inflammatory Pain

Injection of complete Freunds adjuvant (CFA) in the hindpaw of the rat has been shown to produce a long-lasting inflammatory condition, which is associated with behavioural hyperalgesia and allodynia at the injection site (Hylden et al., Pain 37: 229-243, 1989) (Blackburn-Munro et al., 2002). Rats (body weight 260-300 g) were given a s.c. injection of CFA (50% in saline, 100 μl, Sigma) into the plantar surface of the hindpaw under brief halothane anaesthesia. After 24 h, they were then tested for hindpaw weight bearing responses, as assessed using an Incapacitance Tester (Linton Instrumentation, UK), (Zhu et al., 2005). The instrument incorporates a dual channel scale that separately measures the weight of the animal distributed to each hindpaw. While normal rats distribute their body weight equally between the two hindpaws (50-50), the discrepancy of weight distribution between an injured and non-injured paw is a natural reflection of the discomfort level in the injured paw (nocifensive behavior). The rats were placed in the plastic chamber designed so that each hindpaw rested on a separate transducer pad. The averager was set to record the load on the transducer over 5 s time period and two numbers displayed represented the distribution of the rat's body weight on each paw in grams (g). For each rat, three readings from each paw were taken and then averaged. Side-to-side weight bearing difference was calculated as the average of the absolute value of the difference between two hindpaws from three trials (right paw reading-left paw reading).

Assessment of thermal hyperalgesia: Baseline and post-treatment withdrawal latencies to a noxious thermal stimulus were measured according to Hargreaves (Hargreaves et al., 1988) using a plantar test analgesia meter (IITC, Woodland Hills, Calif., model #336). The stimulus intensity was set at 30% of maximum output and the cut-off time was set at 30 seconds. Rats were placed on a glass plate warmed to 28° C. and allowed to habituate to the testing chambers for a minimum of 15 minutes prior to each testing session. The thermal stimulus was applied to the plantar surface of the paw, and the mean latency of three readings on each paw was used as the latency value for each time point. Thermal thresholds were defined as the latency in seconds to the first pain behavior, which includes nocifensive paw withdrawal, flinching, biting and/or licking of the stimulated paw. The mean and standard error of the mean (SEM) were determined for the injured and normal paws for each treatment group.

Hindpaw weight bearing responses were measured in male Sprague-Dawley rats for 2-3 days prior to being given a hindpaw injection of CFA. Twenty-four hours later baseline responses were measured and rats were then administered Compound A (5, 10 and 20 mg/kg, i.p.), Compound H (10, 30 and 60 mg/kg) and morphine (3, 6 and 10 mg/kg). Weight bearing responses were then measured at 30, 60 and 120 min after drug or vehicle injection (data shown at 60 min.). Compounds A and H as well as morphine produced a marked dose-dependent attenuation in the CFA-induced change in weight bearing compared with vehicle. All groups n=7-8.

### Example 7

### Cloning and Expression of ASICs

The cDNA for ASIC1a and ASIC3 can be cloned from rat poly(A)⁺ mRNA and put into expression vectors according to Hesselager et al. (J Biol. Chem. 279 (12):11006-15 2004). All constructs are expressed in CHO-K1 cells (ATCC no. CCL61) or HEK293 cells. CHO-K1 cells are cultured at 37° C. in a humidified atmosphere of 5% CO₂ and 95% air and passaged twice every week. The cells are maintained in DMEM (10 mM HEPES, 2 mM glutamax) supplemented with 10% fetal bovine serum and 2 mM L-proline (Life Technologies). CHO-K1 cells are co-transfected with plasmids containing ASICs and a plasmid encoding enhanced green fluorescent protein (EGFP) using the lipofectamine PLUS transfection kit (Life Technologies) or Lipofectamine 2000 (Invitrogen) according to the manufacturer's protocol. For each transfection it is attempted to use an amount of DNA that yield whole-cell currents within a reasonable range (0.5 nA-10 nA), in order to avoid saturation of the patch-clamp amplifier (approximately 50 ng for ASIC1a and ASIC3). Electrophysiological measurements are performed 16-48 hours after transfection. The cells are trypsinized and seeded on glass coverslips precoated with poly-D-lysine, on the day the electrophysiological recordings were performed.

### Example 8

### General Synthesis Schemes for Compound A

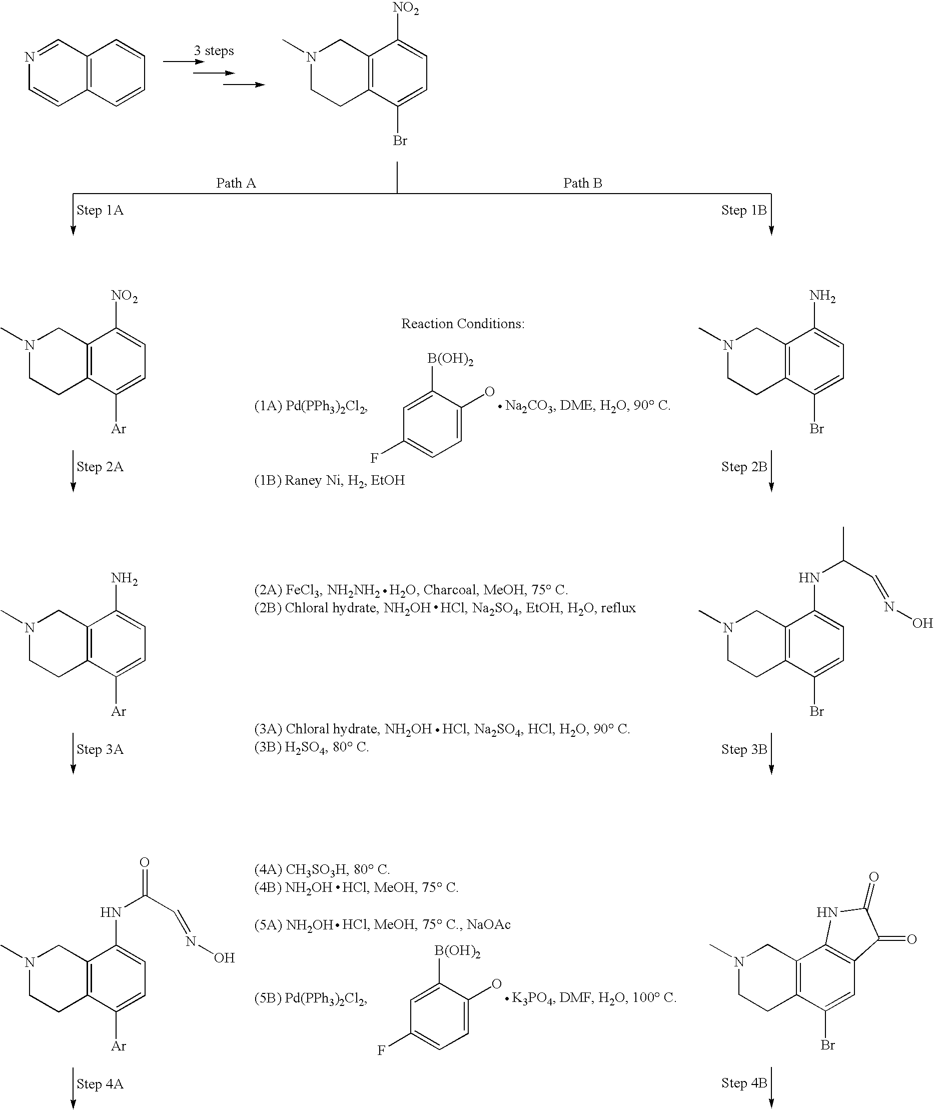

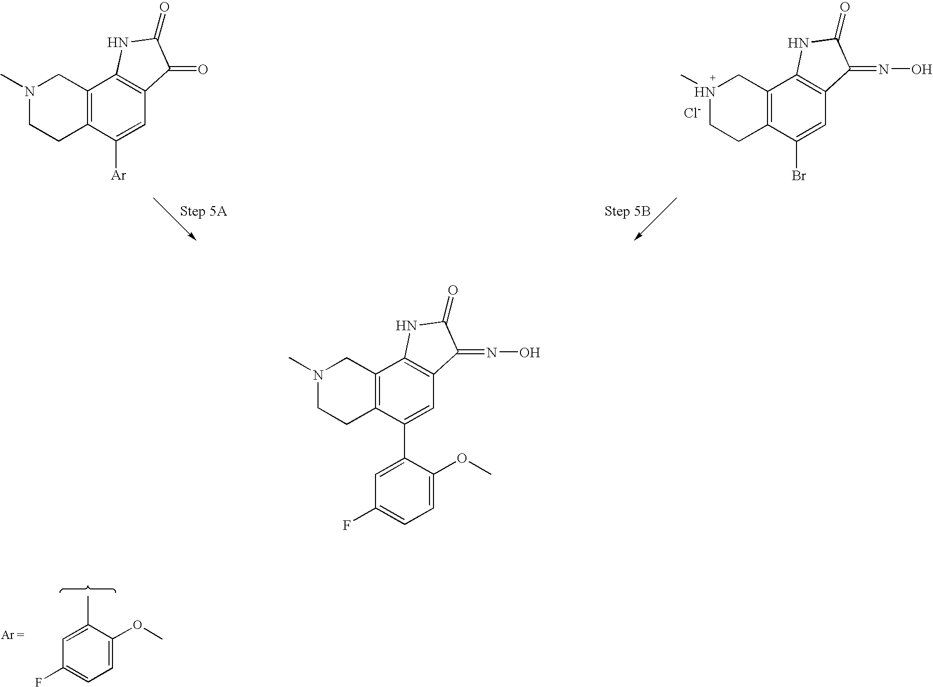

### Example 9

### Synthesis Procedure for Compound A (Path A)

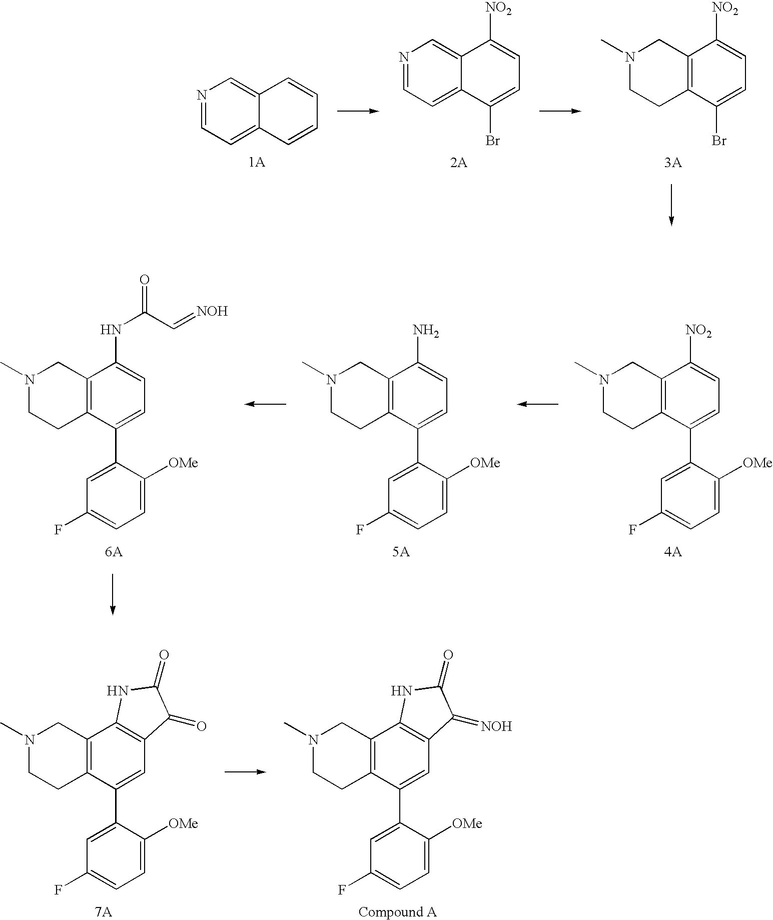

### 5-Bromo-8-nitroisoquinoline (2A)

5-Bromo-8-nitroisoquinoline was prepared from the corresponding isoquinoline (2A) according to the procedure found in William Dalby Brown and Alex Haahr Gouliaev, Organic Syntheses Vol. 81, p 98.

### 5-Bromo-1,2,3,4-tetrahydro-2-methyl-8-nitroisoquinoline (3A)

Compound 2A (340 g, 1.34 mol) in acetone under nitrogen was refluxed until all the starting material was dissolved. The heating was switched off. Dimethyl sulphate (203.3 g, 1.61 mol) was added drop-wise with stirring. After the addition, the reaction mixture was refluxed at 55° C. for 12 hours. The reaction mixture was cooled to room temperature and filtered. TLC revealed no starting material. The solid was washed with cold acetone and dried to get off-white solid (460 g). The intermediate (460 g) was dissolved in acetic acid (1.1 L) under nitrogen and cooled to 15° C. using ice-water bath. Sodium borohydride (81.2 g, 2.15 mol) was added portionwise during a period of 30 minutes. The reaction mixture was stirred at room temperature for 12 hours. TLC revealed no starting material. The reaction mixture was quenched with 5 liters ice, neutralized with aqueous ammonia solution to pH 9.8 and filtered. The solid was washed with water and dried to get product 3A as brownish solid (350 g, 96%).

### 5-(5-Fluoro-2-methoxyphenyl)-2-methyl-8-nitro-1,2,3,4-tetrahydroisoquinoline (4A)

The compound 3A (12.5 g, 46 mmol), 5-fluoro-2-methoxyphenylboronic acid (10.18 g, 59.9 mmol) and sodium carbonate (24.5 g, 230 mmol) were added in a mixture of 1,2-dimethoxy ethane and water (2:1, 180 mL) under nitrogen. It was degasified by passing nitrogen through the solution for 15 minutes. Bis(triphenylphosphine) palladium(II) dichloride was added to this solution. The reaction was covered with aluminum foil and heated at 90° C. for 1 hour. TLC revealed no starting material. The reaction mixture was diluted with water, and then extracted with ethyl acetate. The combined organic layers were washed with water and brine solution, and evaporated to give crude material. The crude product was purified by column chromatography using 60-120 mesh silica gel and eluted at 12% ethyl acetate in petroleum ether to get product 4A (11.0 g, 75%) as yellow solid.

### 5-(5-Fluoro-2-methoxyphenyl)-2-methyl-1,2,3,4-tetrahydroisoquinolin-8-amine (5A)

Compound 4A (11 g, 15.8 mmol) was dissolved in methanol (110 mL). Ferric chloride (550 mg, 5% w/w) and charcoal (1.1 g, 10% w/w) were added the reaction mixture. The mixture was then heated to reflux. Hydrazine hydrate (11 mL) was added drop-wise during reflux. The heating was continued for 8 hours until TLC showed no starting material. The reaction mixture was filtered through celite, and then the filtrate was evaporated. The residue was dissolved in ethyl acetate, washed with water and brine, dried over anhydrous Na₂SO₄, concentrated to get yellow solid 5A (10.0 g, 99%).

### N-(5-(5-Fluoro-2-methoxyphenyl)-2-methyl-1,2,3,4-tetrahydroisoquinolin-8-yl)-2-(hydroxyimino)acetamide (6A)

To a suspension of compound 5A (4.5 g, 15 mmol) in water (23 mL) was added hydrochloric acid (conc., 5.4 mL) drop-wise under stirring. A solution of chloral hydrate (3.9 g, 23 mmol) and sodium sulfate (17.8 g, 125 mmol) in water (99 mL) was added to the solution. Then a solution of hydroxyl amino hydrochloride (4.36 g, 63 mmol) in water (27 mL) was added to the mixture. The reaction mixture was heated at 90° C. for 1 hour. TLC revealed no starting material. The reaction mixture was cooled to 40° C., neutralized with NaHCO₃ solution (10%) to pH 6.5, and extracted with ethyl acetate. The organic layer was washed with water and brine solution and evaporated to get a yellowish solid 6A (5 g).

### 5-(5-Fluoro-2-methoxyphenyl)-8-methyl-6,7,8,9-tetrahydro-1H-pyrrolo[3,2-h]isoquinoline-2,3-dione (7A)

Methanesulphonic acid (5 mL) was heated to 50° C. Compound 6A (1 g, 2.8 mmol) was added portion-wise to this hot methanesulfonic acid under stirring. After the addition, the reaction mixture was heated at 80° C. for one hour. The reaction mixture cooled to 40° C., neutralized with 10% NaHCO₃ solution to pH 7, and extracted with ethyl acetate. The organic layer washed with water, dried over anhydrous Na₂SO₄, and evaporated to get product 7A (800 mg).

### 5-(5-Fluoro-2-methoxyphenyl)-6,7,8,9-tetrahydro-3-(hydroxyimino)-8-methyl-1H-pyrrolo[3,2-h]isoquinolin-2(3H)-one (Compound A)

Compound 7A (800 mg, 2.4 mmol) was dissolved in methanol (8 mL). Hydroxylamine hydrochloride (327 mg, 4.8 mmol) and sodium acetate (2 eqv.) was added, and the reaction was heated at 75° C. for one hour. The reaction mixture was filtered to get yellowish solid Compound A.

### 5-(5-Fluoro-2-methoxyphenyl)-6,7,8,9-tetrahydro-3-(hydroxyimino)-8-ethyl-1H-pyrrolo[3,2-h]isoquinoline-2(3H)-one (Compound B)

Compound B is prepared using the procedure for Compound A by replacing dimethyl sulphate with diethyl sulfate when preparing Compound 3A.

### Example 10

### Synthesis Procedure for Compound A (Path B)

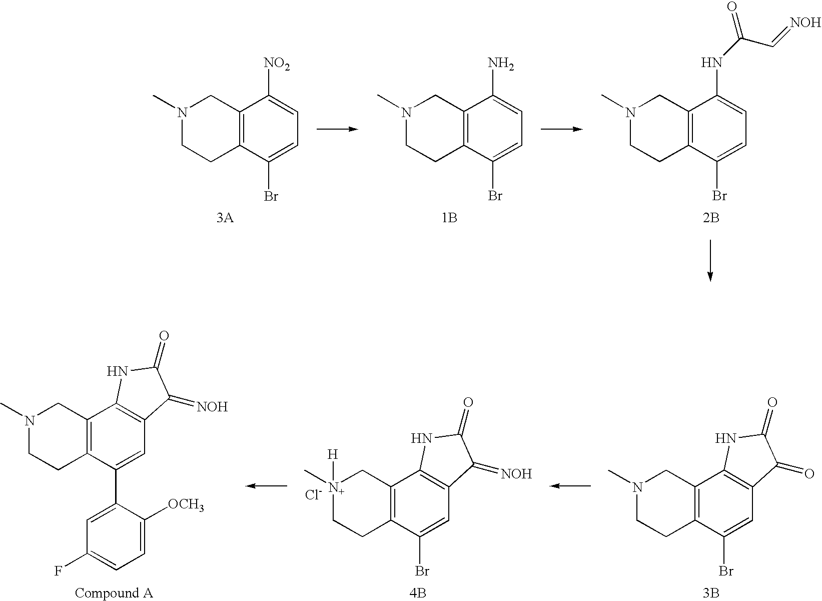

### 5-Bromo-1,2,3,4-tetrahydro-2-methyl-8-nitroisoquinoline (3A)

The synthesis of compound 3A is described in the Path A approach

### 5-Bromo-1,2,3,4-tetrahydro-2-methylisoquinolin-8-amine (1B)

To a solution of N-methyl-5-bromo-8-nitro-1,2,3,4-tetrahydroisoquinoline (4.7 g, 17.3 mmol) in ethanol (50 mL), Raney Nickel (solution in water, 1.5 g) was added. The reaction mixture was stirred at room temperature overnight under H₂. The mixture was filtered through celite and solvent was removed under vacuum to give product 1B.

### N-(5-Bromo-1,2,3,4-tetrahydro-2-methylisoquinolin-8-yl)-2-(hydroxyimino)acetamide (2B)

A mixture of 5-bromo-1,2,3,4-tetrahydro-2-methylisoquinolin-8-amine 1B (3.25 g, 13.5 mmol), chloral hydrate (2.3 g), hydroxylamine hydrochloride (2.9 g), Na₂SO₄ (12 g) in H₂O:EtOH (3:1, 50 mL) was refluxed for 1 hour. The reaction was cooled to 60° C. and carefully basified with 4N NaOH to pH 7. The solid was collected by filtration, washed with water, and dried under vacuum to give product 2B.

### 5-Bromo-6,7,8,9-tetrahydro-8-methyl-1H-pyrrolo[3,2-h]isoquinoline-2,3-dione (3B)

To preheated sulphuric acid (20 mL, 70° C.), N-(5-bromo-1,2,3,4-tetrahydro-2-methylisoquinolin-8-yl-2-(hydroxyimino)acetamide 2B (3.5 g) was added portion-wise over a period of 30 min. The heating was continued further for 1 hr. The reaction mixture was cooled to room temperature and quenched by pouring over ice cold water (100 mL) and then neutralized with aqueous ION NaOH. The precipitated product was filtered and washed with water to give isatin 3B.

### 5-Bromo-6,7,8,9-tetrahydro-3-(hydroxyimino)-8-methyl-1H-pyrrolo[3,2-h]isoquinoline-2(3H)-one hydrochloride (4B)

To the solution of isatin 3B (3.5 g) in methanol (50 ml), hydroxylamine hydrochloride (2.0 g) was added and mixture was refluxed 1 hour. The reaction mixture was cooled to room temperature and solid was collected by filtration, washed with ethanol and ether, and dried under vacuum to give product 4B.

### 5-(5-Fluoro-2-methoxyphenyl)-6,7,8,9-tetrahydro-3-(hydroxyimino)-8-methyl-1H-pyrrolo[3,2-h]isoquinolin-2(3H)-one (Compound A)

A mixture of 5-bromo-6,7,8,9-tetrahydro-3-(hydroxyimino)-8-methyl-1H-pyrrolo[3,2-h]isoquinolin-2(3H)-one 4B (100 mg), 5-fluoro-2-methoxyphenylboronic acid (60 mg), potassium phosphate (72 mg), dichlorobis(triphenylphosphine) palladium(II) (11 mg), water (1.5 mL) and DMF (3 mL) was heated at 100° C. overnight. The solvent was evaporated under vacuum and residue was chromatographed on silica gel (4% to 15% MeOH/CH₂Cl₂) to give 5-(5-fluoro-2-methoxyphenyl)-6,7,8,9-tetrahydro-3-(hydroxyimino)-8-methyl-1H-pyrrolo[3,2-h]isoquinolin-2(3H)-one.

### Example 11

### Synthesis of ASIC Imaging Agents (Compound A) [Scheme 1]

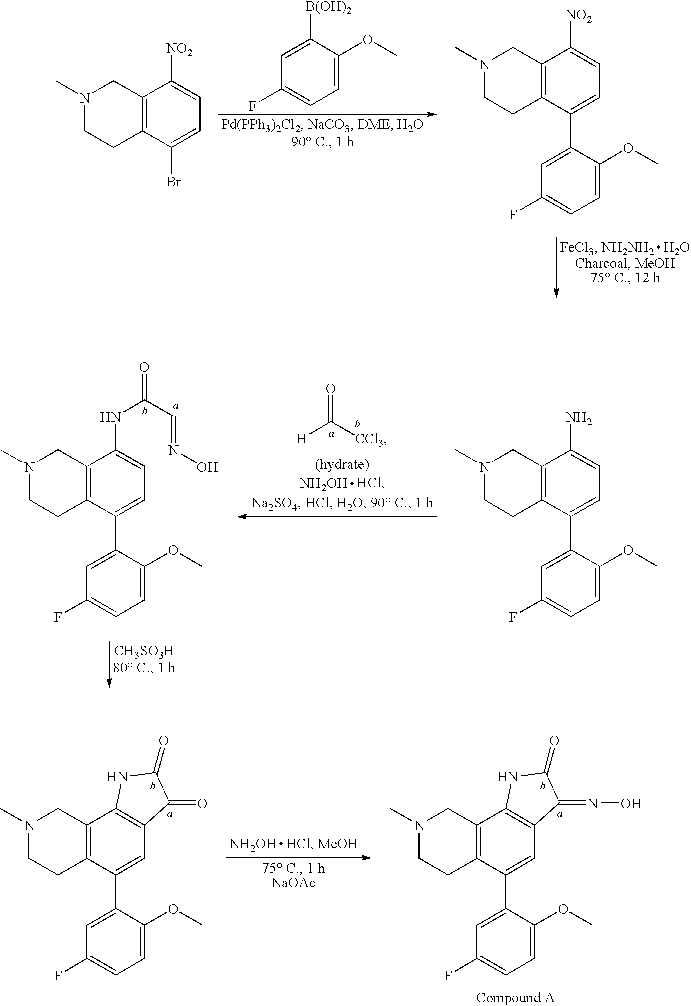

Wherein if carbon “a” or “b” of 2,2,2-trichloroacetaldehyde is ¹⁴C, carbon “a” or “b” of Compound A will be ¹⁴C.

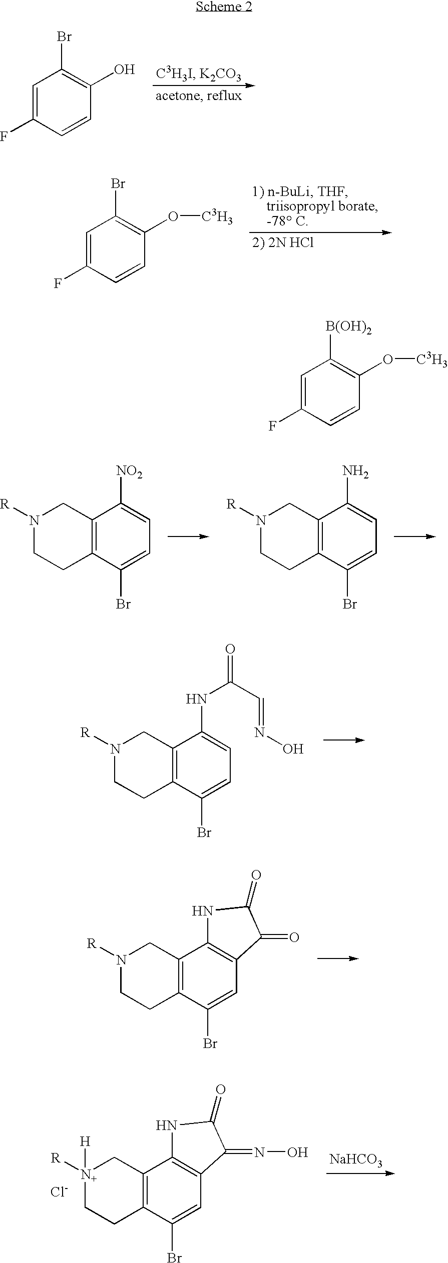

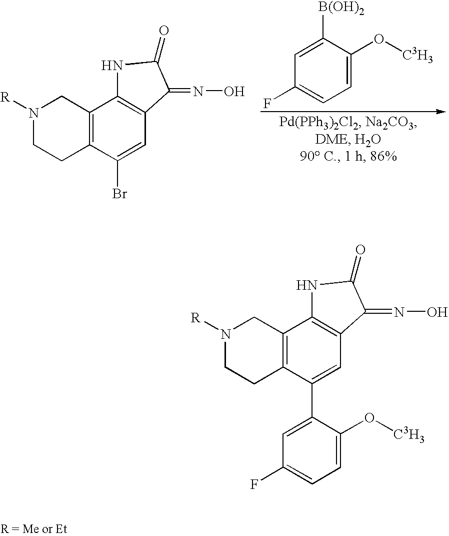

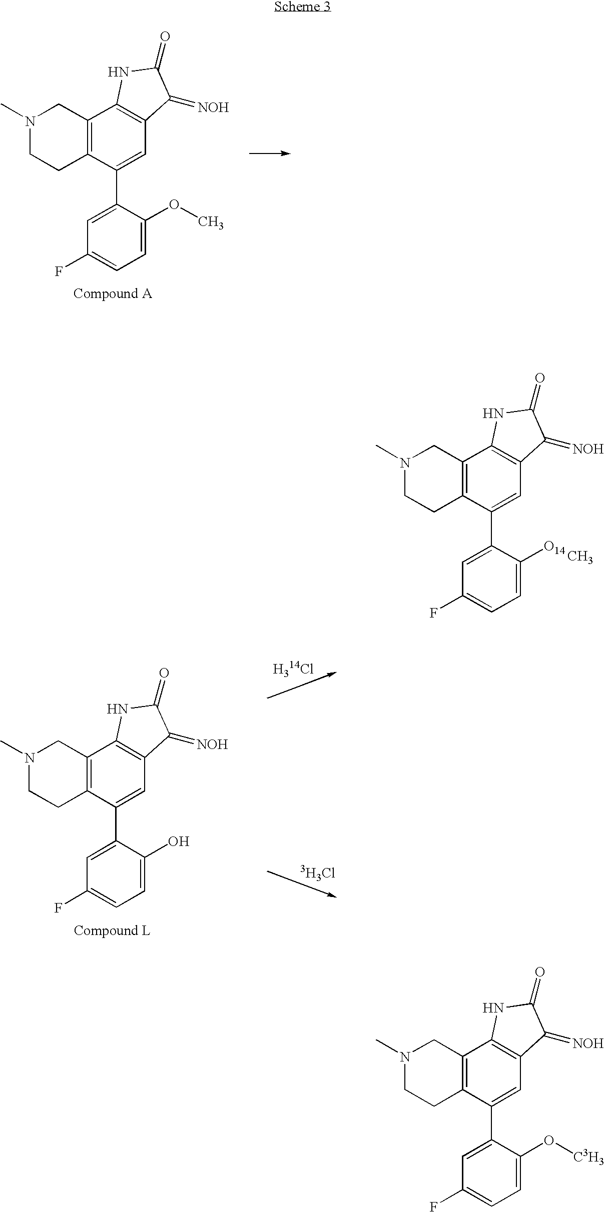

### 5-(5-Fluoro-2-hydroxyphenyl)-3-(hydroxyimino)-8-methyl-6,7,8,9-tetrahydro-1H-pyrrolo[3,2-h]isoquinolin-2(3H)-one (Compound L)

To a mixture of compound A (150 mg, 0.42 mmol) in dry CH₂Cl₂ (10 mL) was added BBr₃ dichloromethane solution (1 M, 1.26 mL). The mixture was stirred at room temperature overnight. The reaction was quenched with saturated NaHCO₃ solution until pH was 7. The yellow solid was filtered and washed with water. The crude product was purified by column chromatography (2% to 20% MeOH/CH₂Cl₂) to give the product, Compound L, as a yellow solid.

This compound can then be further radiolabelled as shown in Scheme 3.

### Example 12

### Isomeric Forms of Compound A

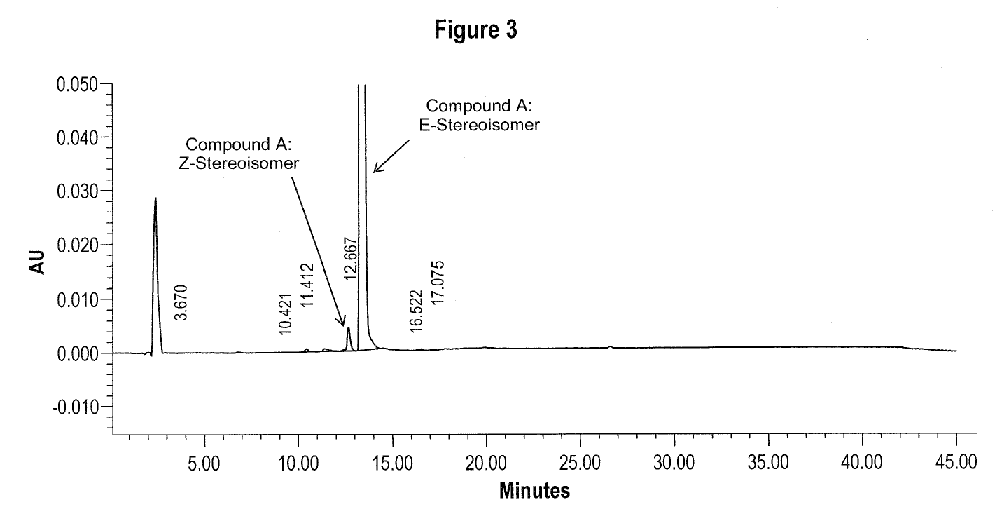

The ═N—OH group of Compound A can exist as 2 stereoisomeric forms: E-(trans) and Z-(cis). Batches produced to date have been shown to be composed predominantly of the E-isomer, as confirmed by the presence of a high purity single peak (97.5% to 99.5% a/a) by HPLC analysis. A minor peak at RRt 0.95 corresponding to the Z-isomer was also detected in these batches. A representative HPLC Chromatogram of the Z-isomer of Compound A is presented in FIG. 3.

A reverse-phase HPLC method, using gradient elution and mobile phases consisting of aqueous ammonium acetate buffer and acetonitrile with detection by UV, was used to determine related compounds and total purity of Compound A. All individual peaks were reported as % a/a. The HPLC chromatographic conditions were set to distinguish between the E and Z stereoisomers.

The IC₅₀ (μM) of the Z form of Compound A (acquired using the procedure of Example 13) is given in the table in Example 3.

### Example 13

### Metabolite Analysis

The objective of this study is to analyze in vitro samples of Compound A and Compound B from rat and human microsomes using liquid chromatography quadrupole ion trap mass spectrometry and identify the nature of the metabolite(s) for each species.

### Instrumentation

The HPLC system consisted of a Thermo Surveror autosampler and a Thermo Surveyor MS pump (San Jose, Calif., USA). The LC-MS/MS system used was a Thermo LCQ Advantage (San Jose, Calif., USA). Data was acquired on a Dell Optiplex desktop computer (Round Rock, Tex., USA) equipped with operation system Windows XP professional. Data acquisition and analysis were performed using XCalibur 1.4 (San Jose, Calif., USA).

### Extraction Procedure

After the incubation period of the liver microsomes, the reaction was stopped with the addition of acetonitrile and frozen for transport. Before analysis, the samples were thawed at room temperature. They were vortexed vigorously and centrifuged at approximately 12000 g for 10 minutes. The supernatant was transferred into an injection vial for analysis.

### Chromatographic Conditions

A gradient mobile phase was used with a Thermo Phenyl 100×2 mm column with particle size of 5 μm. The initial mobile phase condition consisted of methanol and 0.1% formic acid solution at a ratio of 20:80, respectively. From 0 to 2 minutes, the ratio was maintained at 20:80. From 2 to 10 minutes, a linear gradient was applied and a ratio of 90:10 was maintained from 10 to 12 minutes. At 12.1 minutes, the mobile phase composition was reverted to 20:80 and the column was allowed to equilibrate for 4 minutes for a total run time of 16 minutes. The flow rate was fixed at 0.25 mL/min.

### Mass Spectrometry Conditions

The mass spectrometer was interfaced with the HPLC system using a pneumatic assisted electrospray ion source. The sheath gas was set to 15 units and the ESI electrode was set to 4000 V (positive mode). The capillary temperature was set at 300° C., and the capillary voltage was set to 13 V. The mass spectrometer was operating in full scan MS mode using one segment analysis (200-800).

### Tandem Mass Spectrometry

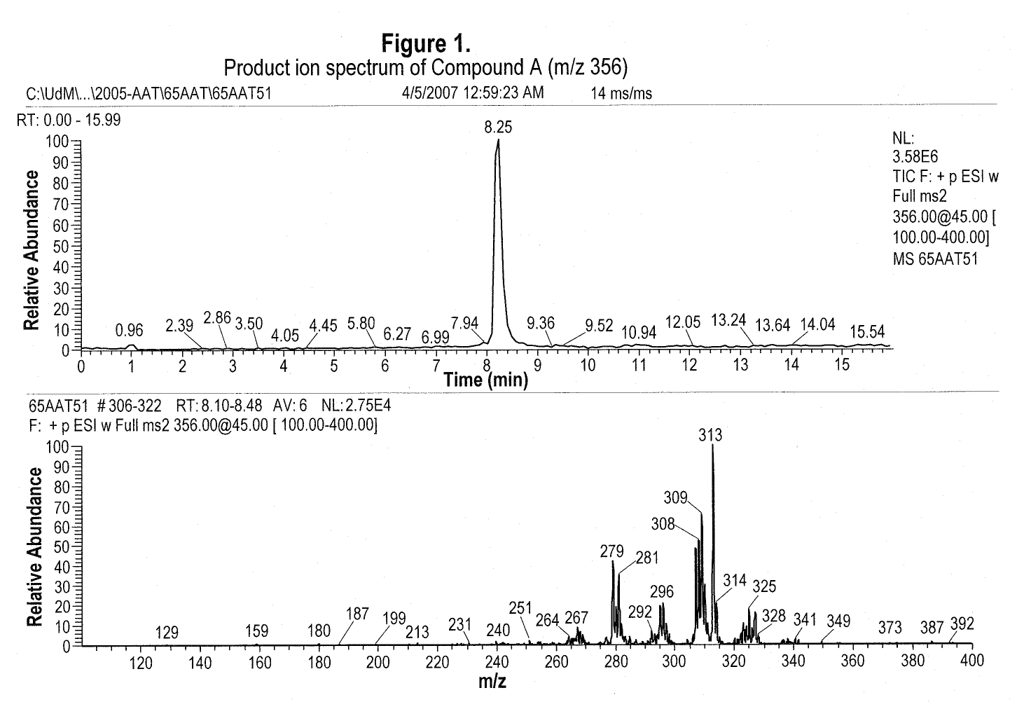

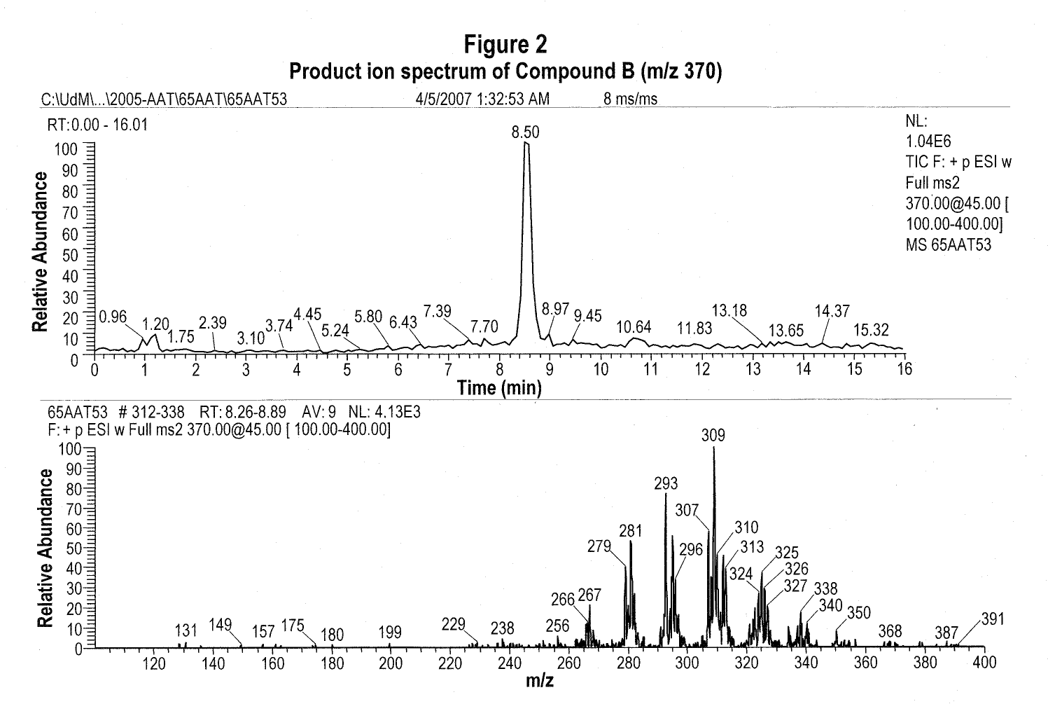

Full scan and product ion mass spectrum for Compound A and Compound B were obtained in positive ion mode. The full scan spectra of Compound A and Compound B contained intense signals for the protonated molecular ion ([M+H]⁺) at m/z 356 and 370, respectively. The product ion spectra generated several fragments compatible with the molecular structure of the molecule. FIG. 1 shows product ion spectrum of Compound A (m/z 356). Scheme A shows a proposed mechanism of fragmentation of Compound A. FIG. 2 shows product ion spectrum of Compound B (m/z 370). Scheme B shows a proposed mechanism of fragmentation of Compound D.

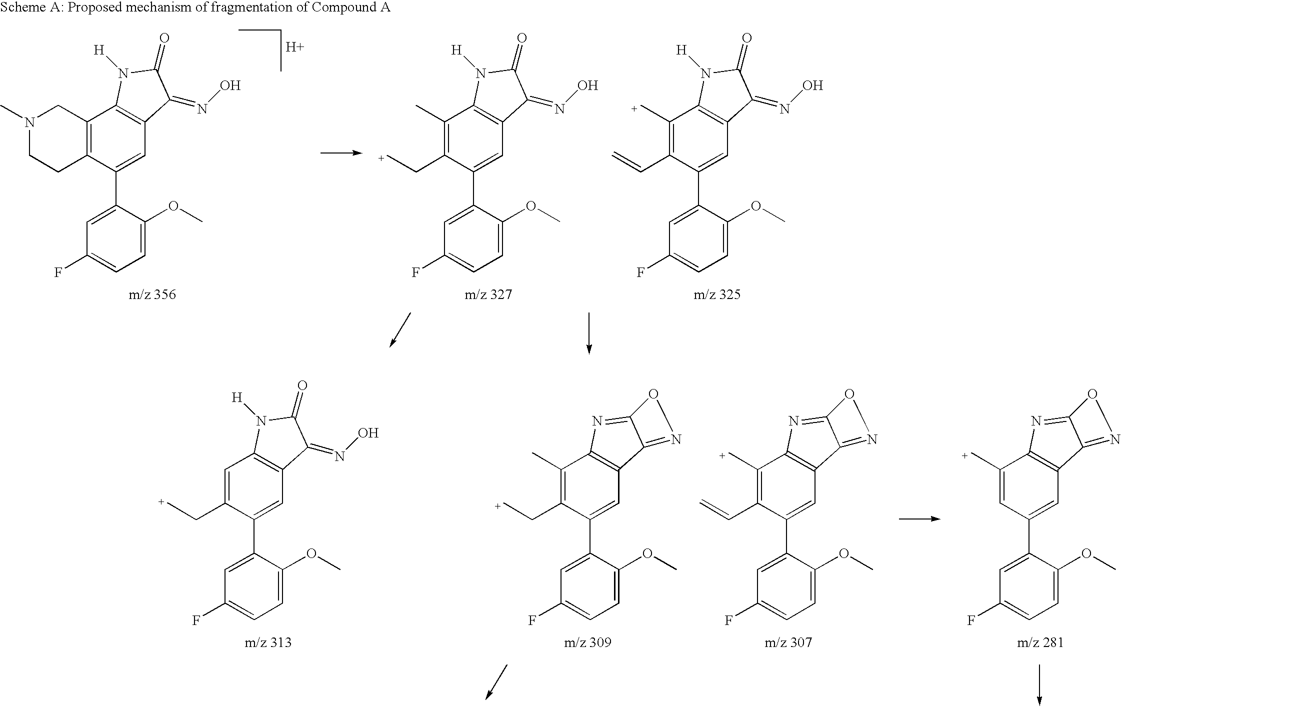

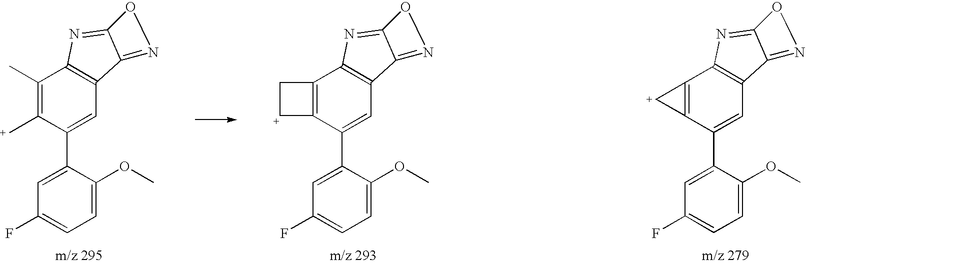

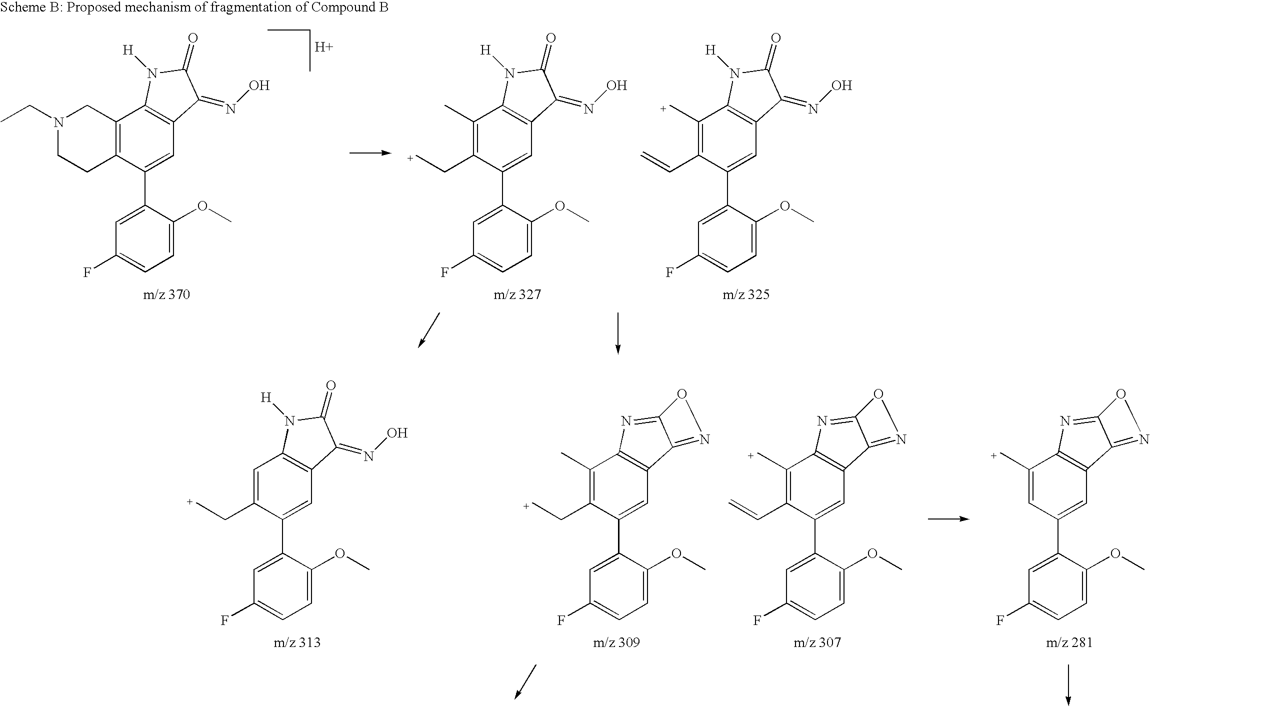

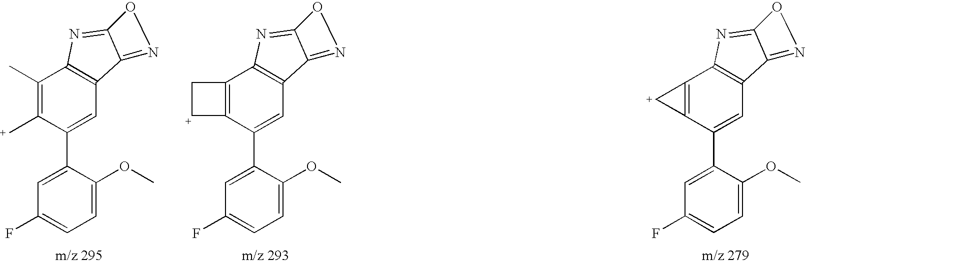

### Rat, Dog and Human Liver Microsomes Stability—Compound A

Rat Microsomes

| | | Calc. Conc. |
|---|---|---|
| Sample ID | Time (min) | (μM) |
| 31 | 0 | 3.61 |
| 32 | 0 | 3.80 |
| 5 | 60 | 2.49 |
| 6 | 60 | 2.67 |
| 7 | 60 | 2.40 |

Dog Microsomes

| | | Calc. Conc. |
|---|---|---|
| Sample ID | Time (min) | (μM) |
| 43 | 0 | 4.71 |
| 44 | 0 | 5.14 |
| 23 | 60 | 3.95 |
| 24 | 60 | 4.05 |
| 25 | 60 | 4.10 |

Human Microsomes

| | | Calc. Conc. |
|---|---|---|
| Sample ID | Time (min) | (μM) |
| 37 | 0 | 4.91 |
| 38 | 0 | 6.54 |
| 14 | 60 | 3.84 |
| 15 | 60 | 3.83 |
| 16 | 60 | 3.77 |

### Rat, Dog and Human Liver Microsomes Stability—Compound B

Rat Microsomes

| | | Calc. Conc. |
|---|---|---|
| Sample ID | Time (min) | (μM) |
| 33 | 0 | 4.95 |
| 34 | 0 | 4.95 |
| 8 | 60 | 0.90 |
| 9 | 60 | 0.92 |
| 10 | 60 | 0.97 |

Dog Microsomes

| | | Calc. Conc. |
|---|---|---|
| Sample ID | Time (min) | (μM) |
| 45 | 0 | 4.44 |
| 46 | 0 | 4.56 |
| 26 | 60 | 4.33 |
| 27 | 60 | 4.38 |
| 28 | 60 | 4.17 |

Human Microsomes

| | | Calc. Conc. |
|---|---|---|
| Sample ID | Time (min) | (μM) |
| 39 | 0 | 4.39 |
| 40 | 0 | 5.04 |
| 17 | 60 | 4.34 |
| 18 | 60 | 4.59 |
| 19 | 60 | 5.03 |

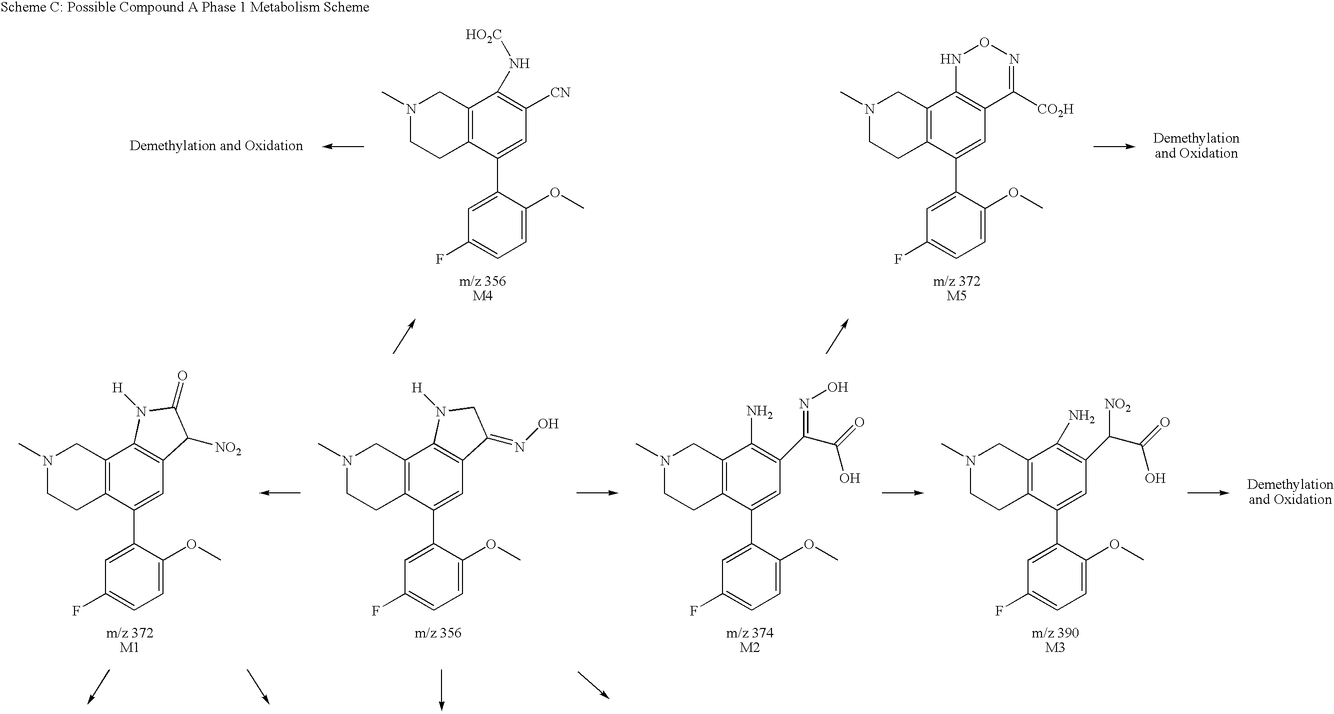

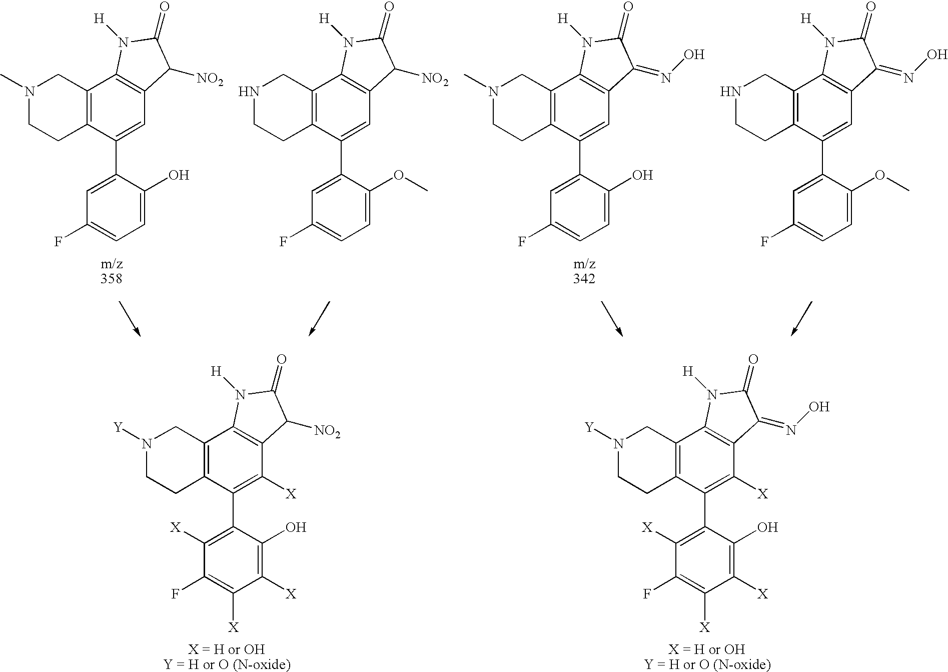

Extracted Ions for Possible Phase 1 Metabolites

| | | | | | desmethyl + | 2(desmethyl) + |
|---|---|---|---|---|---|---|
| Compound | [M + H] | oxidation | desmethyl | 2(desmethyl) | Oxid. | Oxid. |
| Parent | 356 | 372 | 342 | 328 | 358 | 344 |
| M1 | 372 | 388 | 358 | 344 | 374 | 360 |
| M2 | 374 | 390 | 360 | 346 | 376 | 362 |
| M3 | 390 | 406 | 376 | 362 | 392 | 378 |
| M4 | 356 | 372 | 342 | 32S | 358 | 344 |
| M5 | 372 | 388 | 358 | 344 | 374 | 360 |

Metabolites with m/z 342, 372 and 412 were observed in microsomes samples.

Compound A metabolite identification results table

| | m/z 356 | | | |
|---|---|---|---|---|
| Sample ID | (parent) | m/z 342 | m/z 372 | m/z 412 |
| 1 | Yes | no | no | no |
| 5 | Yes | Yes | Yes | trace |
| 6 | Yes | Yes | Yes | trace |
| 7 | Yes | Yes | Yes | trace |
| 14 | Yes | Yes | Yes | no |
| 15 | Yes | Yes | Yes | no |
| 16 | Yes | Yes | Yes | no |
| 23 | Yes | weak | Yes | no |
| 24 | Yes | weak | Yes | no |
| 25 | Yes | weak | Yes | no |

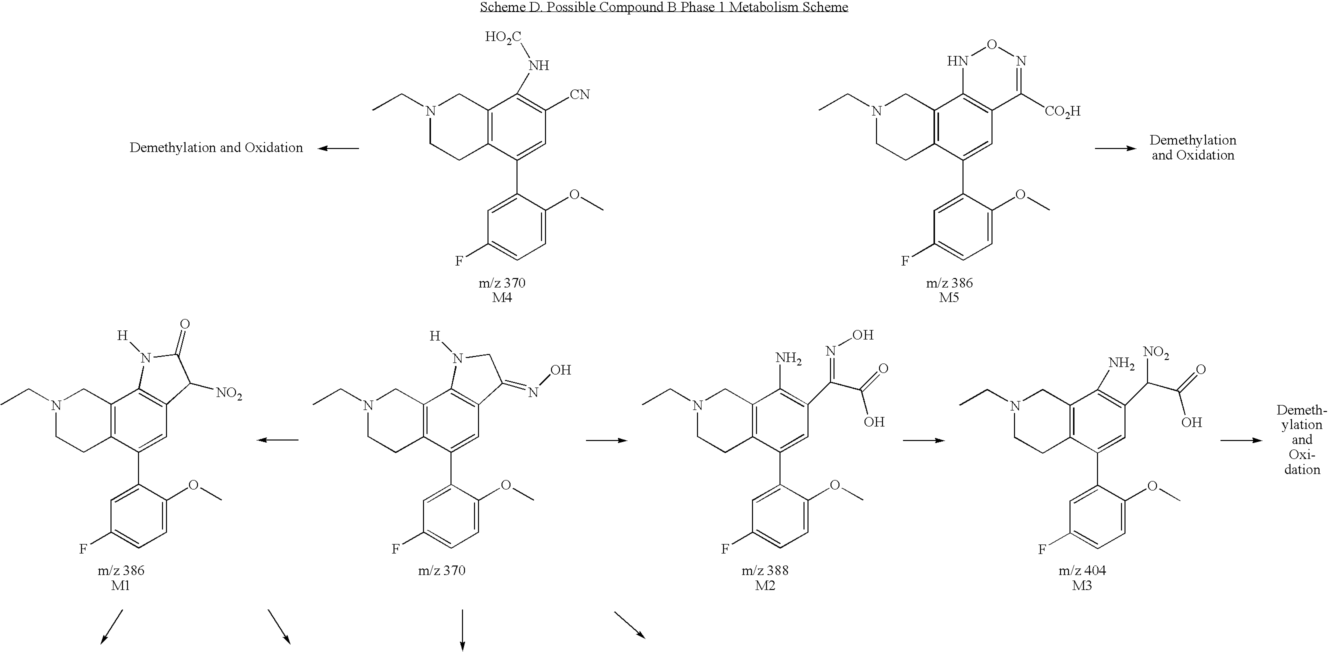

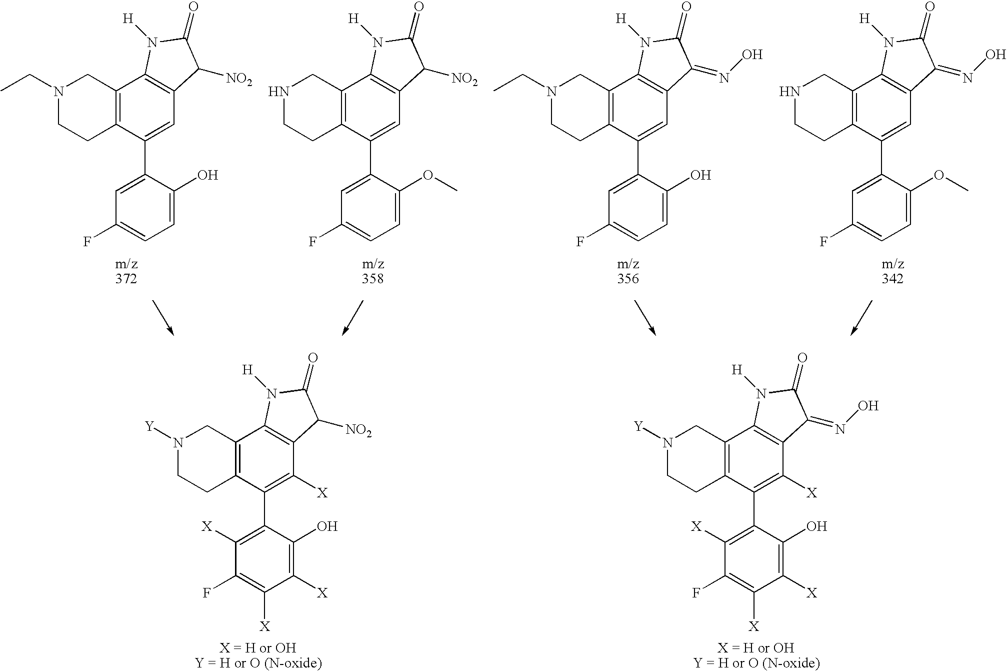

Extracted Ions for Possible Phase 1 Metabolites

| | | | | | desmethyl + | 2(desmethyl) + |
|---|---|---|---|---|---|---|
| Compound | [M + H] | oxidation | desmethyl | 2(desmethyl) | Oxid. | Oxid. |
| Parent | 370 | 386 | 356 | 342 | 372 | 358 |
| M1 | 386 | 402 | 372 | 358 | 388 | 374 |
| M2 | 388 | 404 | 374 | 360 | 390 | 376 |
| M3 | 404 | 420 | 390 | 376 | 406 | 392 |
| M4 | 370 | 386 | 356 | 342 | 372 | 358 |
| M5 | 386 | 402 | 372 | 358 | 338 | 374 |

Compound B metabolite identification results table

| | m/z 370 | | | |
|---|---|---|---|---|
| Sample ID | (parent) | m/z 342 | m/z 356 | m/z 386 |
| 8 | Yes | Yes | Yes | Yes |
| 9 | Yes | Yes | Yes | Yes |
| 10 | Yes | Yes | Yes | Yes |
| 17 | Yes | Yes | low | Yes |
| 18 | Yes | Yes | low | Yes |
| 19 | Yes | Yes | low | Yes |
| 26 | Yes | very weak | very weak | low |
| 27 | Yes | very weak | very weak | low |
| 28 | Yes | very weak | very weak | low |

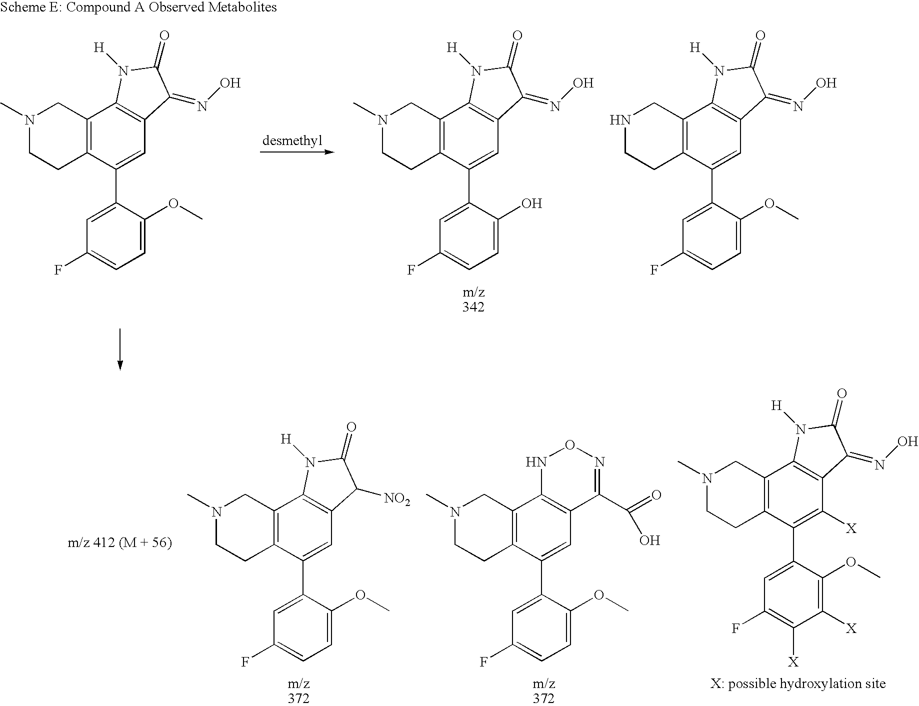

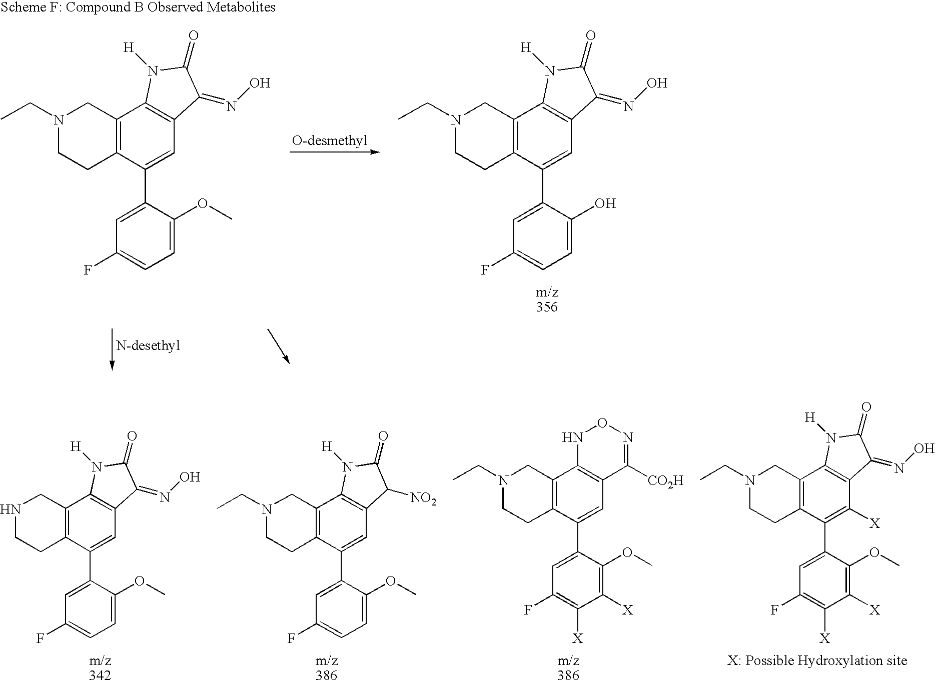

Identification of Predominant Metabolites for Compounds A and B: In Vitro in Microsomal Preparations from Rat, Human and Dog.
% Peak area (relative to parent at T0) for Metabolites after 60 min incubation with microsomes

Compound A

| | Rat | Human | Dog |
|---|---|---|---|
| % N-des methylation (342-313) | 31.88 | 6.64 | 0.34 |
| % O-des methylation (342-299) | 0.63 | 1.21 | 0.65 |
| % Oxidation (372-329) | 0.25 | 0.23 | 1.75 |
| Total % metabolites | 32.76 | 8.08 | 2.74 |

% Peak area (relative to parent at T0) for Metabolites after 60 min incubation with microsomes

Compound B

| | Rat | Human | Dog |
|---|---|---|---|
| % O-des methylation (356-58) | 1.28 | 2.94 | 0.61 |
| % N-des ethylation (342-313) | 6.58 | 1.51 | 0.16 |
| % Oxidation (386-58) | 4.94 | 4.95 | 4.16 |
| Total % metabolites | 12.8 | 9.4 | 4.93 |

### Example 14

### Plasma Composition Analysis—Parent Compound and Metabolites

Rats receive compounds (5 mg/kg) dissolved in HPβCD 15%, dextrose 2.5%, pH 5 by intravenous (i.v). route or (25-30 mg/kg) dissolved in HPβCD 15%, dextrose 2.5%, pH by subcutaneous (s.c.) route or (50-150 mg/kg) dissolved in HPβCD 15%, dextrose 2.5%, pH 5 by oral (p.o.) route. After the treatment, approximately 100 μl of blood sample is collected and the accurate bleeding time is recorded for each sample. Each blood sample is collected into Microvette blood collection tube containing anticoagulant lithium heparin. Following collection, the samples are centrifuged at 5000 for 15 minutes (to avoid haemolysis) and the plasma obtained from each is recovered and stored frozen (at −80° C.) pending analysis. Frozen samples are shipped on dry ice to quantify drug concentration within plasma after extraction procedure as previously described in Example 13.

Plasma composition: Parent compound and metabolites based on
MS identification

| | Compound A | | | Compound B | | |
|---|---|---|---|---|---|---|
| | IV^{a} | SC | PO^{b} | | SC | PO |
| Plasma | (5 | (30 | (50 | IV | (25 | (50 |
| Component | mg/kg) | mg/kg) | mg/kg) | (5 mg/kg) | mg/kg) | mg/kg) |
| Parent (P) | Major | Major | Major | Major | Major | Major |
| N-desMe or | Small | ND | Small | Trace | Trace | Trace |
| desEt | | | | | | |
| O-desMe | Trace | ND | Trace | Trace | Trace | Trace |
| P—OH | ND | ND | ND | Trace | Trace | Trace |

ND = Not detected
^{a}Complete plasma level curves for parent and metabolite
^{b}Profile was similar at 150 mg/kg

Plasma composition: Parent compound and
metabolites based on MS identification

| | Compound G | | Compound J | |
|---|---|---|---|---|
| Plasma | IV | PO | IV | PO |
| Component | (5 mg/kg) | (50 mg/kg) | (5 mg/kg) | (50 mg/kg) |
| Parent (P) | Major | Major | Major | Major |
| N-desEt | Small | Small | Small | Small |
| O-desMe | Small | Small | Small | Small |
| P-OH | Small ^{a} | Small ^{a} | Small | Small |

^{a} Detected but not confirmed

### EQUIVALENTS

The invention has been described herein with reference to certain examples and embodiments only. No effort has been made to exhaustively describe all possible examples and embodiments of the invention. Indeed, those of skill in the art will appreciate that various additions, deletions, modifications and other changes may be made to the above-described examples and embodiments, without departing from the intended spirit and scope of the invention as recited in the following claims. It is intended that all such additions, deletions, modifications and other changes be included within the scope of the following claims.

### INCORPORATION BY REFERENCE

The entire contents of all patents, published patent applications, websites, and other references cited herein are hereby expressly incorporated herein in their entireties by reference.

1. A metabolite having the Formula III: 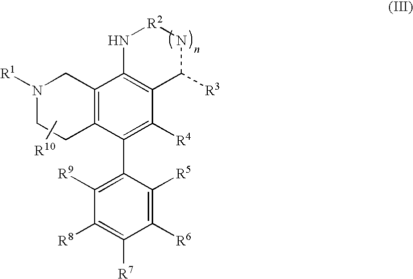
and pharmaceutically acceptable salts, enantiomers, stereoisomers, rotamers, tautomers, diastereomers, or racemates thereof;
wherein
R¹ is hydrogen or C₁-C₄ alkyl;
R² is O or C(O);
n is 0 or 1;
the dashed lines, independently, indicate a single or double bond;
R³ is ═NOH, —NO₂ or CO₂H;
R⁴, R⁵, R⁶, R⁷, R⁸ and R⁹ are each, independently, H, halogen, OH, CO₂H, C₁-C₄ alkyl, C₁-C₄ alkoxyl, hydroxy substituted C₁-C₄ alkyl, a C₁-C₄ aldehyde or a C₁-C₄ carboxylic acid; and
R¹⁰ is H, OH or ═O.

2. The metabolite of claim 1, wherein R¹ is hydrogen, methyl or ethyl, R⁴ and R⁹ are each, independently, H or OH, R⁵ is H, CH₃, OCH₃, OCH₂CH₃, CH₂OH, OH, C(O)H or CO₂H, R⁶ is H, Cl, CH₃, CH₂OH, OH, C(O)H or CO₂H, R⁷ is H, F, OH or SO₂NMe₂, R⁸ is H, F, Cl, I, OH, CH₃, CH₂OH, C(O)H, CO₂H, and R¹⁰ is H.

3. A metabolite having the Formula IV: 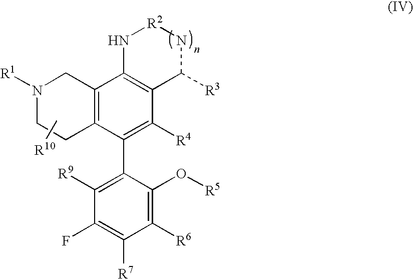
and pharmaceutically acceptable salts, enantiomers, stereoisomers, rotamers, tautomers, diastereomers, or racemates thereof;
wherein
R¹ is hydrogen, methyl or ethyl;
R² is O or C(O);
n is 0 or 1;
the dashed lines, independently, indicate a single or double bond;
R³ is ═NOH, —NO₂ or CO₂H;
R⁵ is H or CH₃;
R⁴, R⁶ and R⁷ are each, independently, selected from the group consisting of H and OH;
R⁹ is H or OH; and
R¹⁰ is H, OH or ═O.

4. The metabolite of claim 3, wherein R⁹ and R¹⁰ are H.

5. The metabolite of claim 3, wherein the metabolite is selected from the group consisting of: 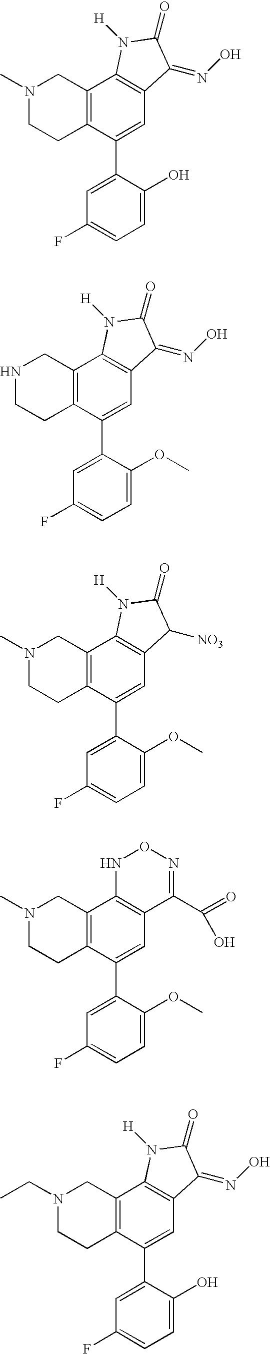 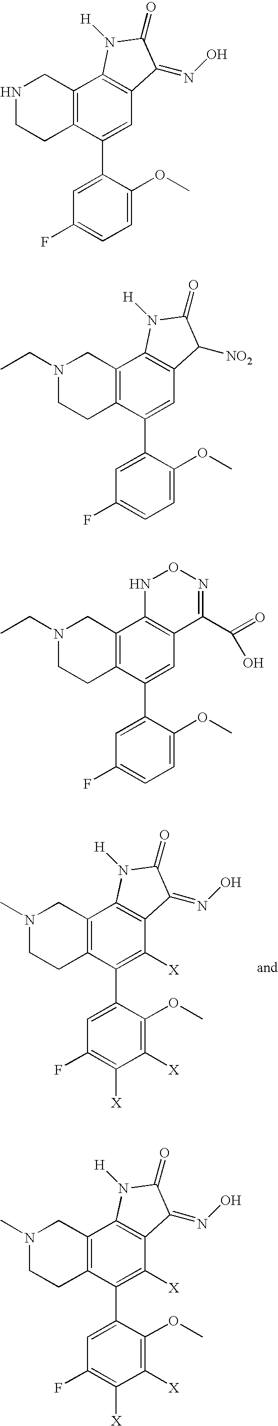
wherein each X is, independently, H or OH.

6-15. (canceled)

16. An ASIC imaging agent of the Formula I: 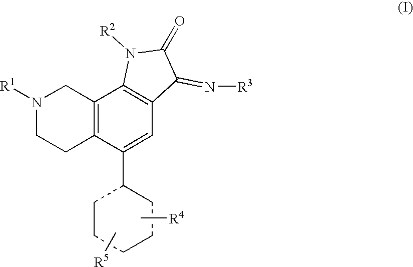
and pharmaceutically acceptable salts, enantiomers, stereoisomers, rotamers, tautomers, diastereomers, or racemates thereof;
wherein
the dashed lines indicate a single or double bond;
R¹ is selected from the group consisting of hydrogen, alkyl, alkoxy-alkyl, hydroxy-alkyl, alkoxy-carbonyl-alkyl, alkyl-carbonyl-oxy-alkyl, cycloalkyl, cycloalkyl-alkyl, alkenyl, alkynyl, alkoxy, sulfonamide, amino, sulfonyl, sulfonic acid, urea, phenyl or benzyl, in which the phenyl or benzyl group is optionally substituted with halogen, CF₃, nitro, amino, cyano, hydroxy-alkyl, alkoxy, sulfonamide, alkenyl, alkynyl, amino, sulfonyl, sulfonic acid and urea;
R² is selected from the group consisting of hydrogen, hydroxyl, alkyl, alkenyl, alkynyl, —(CH₂)₁₋₄S(O)₃H, —C(O)C₁₋₄alkyl and —S(O)₂C₁₋₄alkyl;
R³ is selected from the group consisting of hydrogen, hydroxyl, alkyl, acyl, phenyl, benzyl, —(CH₂)₁₋₄COOH, —C(O)N(CH₃)₂, —O-phenyl, —OCF₃, alkoxy, —O(CH₂)₀₋₄OCH₃, —C(O)H, —C(O)CH₃, 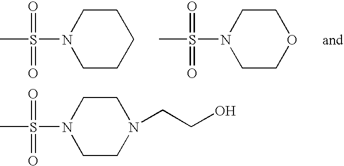
and R⁴ and R⁵ are each, independently, selected from the group consisting of hydrogen, halogen, CF₃, nitro, amino, cyano, hydroxyl, alkyl, alkoxy, phenoxy and phenyl, or a group of the formula —SO₂NR′R″, wherein R′ and R″ independently of each another represents hydrogen or alkyl;
wherein at least one of the atoms of Formula I is an isotope.

17. The ASIC imaging agent of claim 16, wherein the compound is represented by the Formula II, 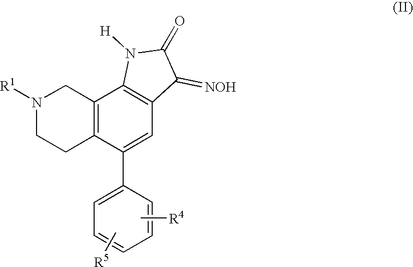
and pharmaceutically acceptable salts, enantiomers, stereoisomers, rotamers, tautomers, diastereomers, or racemates thereof;
wherein
R¹ is selected from the group consisting of hydrogen, alkyl, alkoxy-alkyl, alkoxy-carbonyl-alkyl, alkyl-carbonyl-oxy-alkyl, cycloalkyl, cycloalkyl-alkyl, alkenyl, alkynyl, alkoxy, sulfonamide, amino, sulfonyl, sulfonic acid, urea phenyl or benzyl, in which the phenyl or benzyl group is optionally substituted with halogen, CF₃, nitro, amino, cyano, hydroxy-alkyl, alkoxy, sulfonamide, alkenyl, alkynyl, amino, sulfonyl, sulfonic acid and urea; and
R⁴ and R⁵ are each, independently, selected from the group consisting of hydrogen, halogen, phenoxy, CF₃, nitro, amino, cyano, hydroxyl, alkyl, alkoxy and phenyl, or a group of the formula —SO₂NR′R″, wherein R′ and R″ independently of each another represents hydrogen or alkyl;
wherein at least one of the atoms of Formula II is an isotope.

18. The imaging agent of claim 17, wherein R¹ is selected from the group consisting of hydrogen, C₁₋₄-alkyl, C₁₋₄-alkenyl, and C₁₋₄-alkynyl; and R⁴ and R⁵ are each, independently, selected from the group consisting of halogen, CF₃, nitro, amino, cyano, hydroxyl, C₁₋₄-alkyl, C₁₋₄-alkoxy, phenoxy and phenyl, wherein at least one of the atoms of Formula II is an isotope.

19. The ASIC imaging agent of claim 16, wherein the isotope is selected from the group consisting of ²H, ³H, ¹³C, ¹⁴C, ¹⁵N, ¹⁸O, ¹⁷O, ³¹P, ³²P, ³⁵S, ¹⁸F, ³⁶Cl, ¹²³I, ¹²⁵I, ¹²⁷I, ¹²⁹I, ¹³⁰I, and ¹³¹I.

20. The ASIC imaging agent of claim 16, wherein the isotope is selected from the group consisting of ³H and ¹⁴C.

21. The ASIC imaging agent of claim 16, wherein the compound is selected from the group consisting of 5-(5-fluoro-2-methoxyphenyl)-6,7,8,9-tetrahydro-3-(hydroxyimino)-8-methyl-1H-pyrrolo[3,2-h]isoquinoline-2(3H)-one (Compound A); 5-(5-fluoro-2-methoxyphenyl)-6,7,8,9-tetrahydro-3-(hydroxyimino)-8-ethyl-1H-pyrrolo[3,2-h]isoquinoline-2(3H)-one (Compound B); 5-(5-chloro-2-methoxyphenyl)-6,7,8,9-tetrahydro-3-(hydroxyimino)-8-methyl-1H-pyrrolo[3,2-h]isoquinoline-2(3H)-one (Compound C); 5-(3,5-dimethylphenyl)-6,7,8,9-tetrahydro-3-(hydroxyimino)-8-methyl-1H-pyrrolo[3,2-h]isoquinoline-2(3H)-one (Compound D); 5-(3,5-dimethylphenyl)-6,7,8,9-tetrahydro-3-(hydroxyimino)-8-ethyl-1H-pyrrolo[3,2-h]isoquinoline-2(3H)-one (Compound E); 5-(2,5-dimethylphenyl)-6,7,8,9-tetrahydro-3-(hydroxyimino)-8-ethyl-1H-pyrrolo[3,2-h]isoquinoline-2(3H)-one (Compound F); 5-(5-chloro-2-methoxyphenyl)-6,7,8,9-tetrahydro-3-(hydroxyimino)-8-ethyl-1H-pyrrolo[3,2-h]isoquinoline-2(3H)-one
(Compound G); 5-(2,3-dimethyl-phenyl)-6,7,8,9-tetrahydro-3-(hydroxyimino)-8-ethyl-1H-pyrrolo[3,2-h]isoquinoline-2(3H)-one (Compound I); 5-phenyl-6,7,8,9-tetrahydro-3-(hydroxyimino)-8-ethyl-1H-pyrrolo[3,2-h]isoquinoline-2(3H)-one (Compound H); 5-(2-methoxy-phenyl)-6,7,8,9-tetrahydro-3-(hydroxyimino)-8-ethyl-1H-pyrrolo[3,2-h]isoquinoline-2(3H)-one (Compound J); 5-(2-ethoxy-phenyl)-6,7,8,9-tetrahydro-3-(hydroxyimino)-8-ethyl-1H-pyrrolo[3,2-h]isoquinoline-2(3H)-one (Compound K); 5-(3-chloro-4-fluoro-phenyl)-6,7,8,9-tetrahydro-3-(hydroxyimino)-8-ethyl-1H-pyrrolo[3,2-h]isoquinoline-2(3H)-one; 5-(5-iodo-2-methoxy-phenyl)-6,7,8,9-tetrahydro-3-(hydroxyimino)-8-methyl-1H-pyrrolo[3,2-h]isoquinoline-2(3H)-one; and 5-(5-iodo-2-methoxy-phenyl)-6,7,8,9-tetrahydro-3-(hydroxyimino)-8-ethyl-1H-pyrrolo[3,2-h]isoquinoline-2(3H)-one, wherein at least one of the atoms of the aforementioned compounds is an isotope.

22. The ASIC imaging agent of claim 16, wherein the imaging agent is selected from the group consisting of

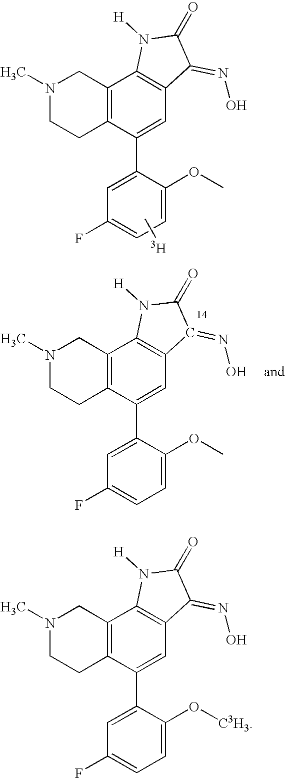

23. The ASIC imaging agent of claim 16, wherein the imaging agent is a ion channel-targeting agent.

24-26. (canceled)

27. A method for diagnosing an ion channel-related condition in a patient, comprising administering an ion channel-targeting imaging agent to said patient, wherein said ion channel-targeting imaging agent is imaged in said patient to determine the activity or amount of an ion channel in said patient.

28-30. (canceled)

31. A method for imaging ion channel-activity in a patient, comprising administering an ion channel-targeting imaging agent to said patient and imaging said ion channel-targeting imaging agent in said patient to determine the activity or amount of an ion channel in said patient.

32. (canceled)

33. A method for diagnosing pain, inflammation, cardiovascular disorders, respiratory conditions, genitourinary disorders, gastrointestinal disorders, or neurological disorders in a patient, comprising administering an imaging agent of Formula I to said patient and said imaging agent is imaged in said patient to determine the presence of one or more of these conditions.

34. A method for imaging a tumor on or in a mammalian tissue inflicted with a tumor comprising contacting the mammalian tissue with an effective amount of an imaging agent of Formula I, and detecting the presence of the imaging agent.

35. A method for imaging a tumor in a subject inflicted with a tumor comprising administering to the mammal an effective amount of an agent of Formula I, and detecting the presence of the imaging agent.

36-45. (canceled)
